SEQUENCE LISTING (56 sequences)

SEQ ID NO: 1 - DNA, 4627 bp, Homo sapiens
  source 1..4627: mol_type = genomic DNA
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgcttttcttttttttttttttttttttttttttttttgagatggagttttgctcttgttgcccaggctggagtgcaatggtgcatcttggctcactgcaacctccgcctcccaggttcaagtgattctcctgcctcagcctcccgagtagctgagattacaggcacccgccaccatgcctggctaattttttgtatttttagtagagacagggtttcactatgttggccaggctggtctcgaactcctgacctcaggtgatccacccgcttcagcctcccaaagtgctgggattacaggcgtgagccaccacacccggcctgcttttcttaaagatcaatctgagtgctgtacggagagtgggttgtaagccaagagtagaagcagaaagggagcagttgcagcagagagatgatggaggcctgggcagggtggtggcagggaggtaaccaacaccattcaggtttcaaaggtagaaccatgcagggatgagaaagcaaagaggggatcaaggaaggcagctggattttggcctgagcagctgagtcaatgatagtgccgtttactaagaagaaaccaaggaaaaaatttggggtgcagggatcaaaactttttggaacatatgaaagtacgtgtttatactctttatggcccttgtcactatgtatgcctcgctgcctccattggactctagaatgaagccaggcaagagcagggtctatgtgtgatggcacatgtggccagggtcatgcaacatgtactttgtacaaacagtgtatattgagtaaatagaaatggtgtccaggagccgaggtatcggtcctgccagggccaggggctctccctagcaggtgctcatatgctgtaagttccctccagatctctccacaaggaggcatggaaaggctgtagttgttcacctgcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccatctctgactttccattctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagttgcacttctcccctatgaggtaggaagaacagttgtttagaaacgaagaaactgaggccccacagctaatgagtggaggaagagagacacttgtgtacaccacatgccttgtgttgtacttctctcaccgtgtaacctcctcatgtcctctctccccagtacggctctcttagctcagtagaaagaagacattacactcatattacaccccaatcctggctagagtctccgcaccctcctcccccagggtccccagtcgtcttgctgacaactgcatcctgttccatcaccatcaaaaaaaaactccaggctgggtgcgggggctcacacctgtaatcccagcactttgggaggcagaggcaggaggagcacaggagctggagaccagcctgggcaacacagggagaccccgcctctacaaaaagtgaaaaaattaaccaggtgtggtgctgcacacctgtagtcccagctacttaagaggctgagatgggaggatcgcttgagccctggaatgttgaggctacaatgagctgtgattgcgtcactgcactccagcctggaagacaaagcaagatcctgtctcaaataataaaaaaaataagaactccagggtacatttgctcctagaactctaccacatagccccaaacagagccatcaccatcacatccctaacagtcctgggtcttcctcagtgtccagcctgacttctgttcttcctcattccagatctgcaagattgtaagacagcctgtgctccctcgctccttcctctgcattgcccctcttctccctctccaaacagagggaactctcctacccccaaggaggtgaaagctgctaccacctctgtgcccccccggcaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctgcagaatgttgtgaggggtttatttttttttaatagtgttcataaagaaatacatagtattcttcttctcaagacgtggggggaaattatctcattatcgaggccctgctatgctgtgtatctgggcgtgttgtatgtcctgctgccgatgccttc

SEQ ID NO: 2 - DNA, 1448 bp, Homo sapiens
  source 1..1448: mol_type = genomic DNA
aggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcagacaagactagatccaaaaagaaaggaaccagcgcacaccatgaaggagaattgggcacctgtggttcattcttctcccagattctcagcccaacagagccaagcagctgggtcccctttctatgtggcctgtgtaactctcatctgggtggtgccccccatccccctcagtgctgccacatgccatggattgcaaggacaatgtggctgacatctgcatggcagaagaaaggaggtgctgggctgtcagaggaagctggtctgggcctgggagtctgtgccaactgcaaatctgactttacttttaattgcctatgaaaataaggtctctcatttattttcctctccctgctttctttcagactgtggctttacctcgggtaagtaagcccttccttttcctctccctctctcatggttcttgacctagaaccaaggcatgaagaactcacagacactggagggtggagggtgggagagaccagagctacctgtgcacaggtacccacctgtccttcctccgtgccaacagtgtcctaccagcaaggggtcctgtctgccaccatcctctatgagatcctgctagggaaggccaccctgtatgctgtgctggtcagcgcccttgtgttgatggccatggtaagcaggagggcaggatggggccagcaggctggaggtgacacactgacaccaagcacccagaagtatagagtccctgccaggattggagctgggcagtagggagggaagagatttcattcaggtgcctcagaagataacttgcacctctgtaggatcacagtggaagggtcatgctgggaaggagaagctggagtcaccagaaaacccaatggatgttgtgatgagccttactatttgtgtggtcaatgggccctactactttctctcaatcctcacaactcctggctcttaataacccccaaaactttctcttctgcaggtcaagagaaaggatttctga

SEQ ID NO: 3 - DNA, 1489 bp, Homo sapiens
  source 1..1489: mol_type = genomic DNA
aggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcggacaagactagatccagaagaaagccagagtggacaaggtgggatgatcaaggttcacagggtcagcaaagcacggtgtgcacttcccccaccaagaagcatagaggctgaatggagcacctcaagctcattcttccttcagatcctgacaccttagagctaagctttcaagtctccctgaggaccagccatacagctcagcatctgagtggtgtgcatcccattctcttctggggtcctggtttcctaagatcatagtgaccacttcgctggcactggagcagcatgagggagacagaaccagggctatcaaaggaggctgactttgtactatctgatatgcatgtgtttgtggcctgtgagtctgtgatgtaaggctcaatgtccttacaaagcagcattctctcatccatttttcttcccctgttttctttcagactgtggcttcacctccggtaagtgagtctctcctttttctctctatctttcgccgtctctgctctcgaaccagggcatggagaatccacggacacaggggcgtgagggaggccagagccacctgtgcacaggtgcctacatgctctgttcttgtcaacagagtcttaccagcaaggggtcctgtctgccaccatcctctatgagatcttgctagggaaggccaccttgtatgccgtgctggtcagtgccctcgtgctgatggccatggtaaggaggagggtgggatagggcagatgatgggggcaggggatggaacatcacacatgggcataaaggaatctcagagccagagcacagcctaatatatcctatcacctcaatgaaaccataatgaagccagactggggagaaaatgcagggaatatcacagaatgcatcatgggaggatggagacaaccagcgagccctactcaaattaggcctcagagcccgcctcccctgccctactcctgctgtgccatagcccctgaaaccctgaaaatgttctctcttccacaggtcaagagaaaggattccagaggctag

SEQ ID NO: 4 - protein, 140 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 5 - protein, 176 aa, Homo sapiens
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 6 - protein, 178 aa, Homo sapiens
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 7 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 8 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 9 - protein, 41 aa, Homo sapiens
RSKRSRLLHSDYMNMTPRRPGPTRKHYQPYAPPRDFAAYRS

SEQ ID NO: 10 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 11 - protein, 42 aa, Homo sapiens
KRGRKKLLYIFKQPFMRPVQTTQEEDGCSCRFPEEEEGGCEL

SEQ ID NO: 12 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 13 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 14 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 15 - DNA, 2733 bp, Homo sapiens
  source 1..2733: mol_type = genomic DNA
ctcgcgaggccggctaggcccgaatgtcgttagccgtggggaaagatggcggaaaatttaaaaggctgcagcgtgtgttgcaagtcttcttggaatcagctgcaggacctgtgccgcctggccaagctctcctgccctgccctcggtatctctaagaggaacctctatgactttgaagtcgagtacctgtgcgattacaagaagatccgcgaacaggaatattacctggtgaaatggcgtggatatccagactcagagagcacctgggagccacggcagaatctcaagtgtgtgcgtatcctcaagcagttccacaaggacttagaaagggagctgctccggcggcaccaccggtcaaagaccccccggcacctggacccaagcttggccaactacctggtgcagaaggccaagcagaggcgggcgctccgtcgctgggagcaggagctcaatgccaagcgcagccatctgggacgcatcactgtagagaatgaggtggacctggacggccctccgcgggccttcgtgtacatcaatgagtaccgtgttggtgagggcatcaccctcaaccaggtggctgtgggctgcgagtgccaggactgtctgtgggcacccactggaggctgctgcccgggggcgtcactgcacaagtttgcctacaatgaccagggccaggtgcggcttcgagccgggctgcccatctacgagtgcaactcccgctgccgctgcggctatgactgcccaaatcgtgtggtacagaagggtatccgatatgacctctgcatcttccggacggatgatgggcgtggctggggcgtccgcaccctggagaagattcgcaagaacagcttcgtcatggagtacgtgggagagatcattacctcagaggaggcagagcggcggggccagatctacgaccgtcagggcgccacctacctctttgacctggactacgtggaggacgtgtacaccgtggatgccgcctactatggcaacatctcccactttgtcaaccacagttgtgaccccaacctgcaggtgtacaacgtcttcatagacaaccttgacgagcggctgccccgcatcgctttctttgccacaagaaccatccgggcaggcgaggagctcacctttgattacaacatgcaagtggaccccgtggacatggagagcacccgcatggactccaactttggcctggctgggctccctggctcccctaagaagcgggtccgtattgaatgcaagtgtgggactgagtcctgccgcaaatacctcttctagcccttagaagtctgaggccagactgactgagggggcctgaagctacatgcacctcccccactgctgccctcctgtcgagaatgactgccagggcctcgcctgcctccacctgcccccacctgctcctacctgctctacgttcagggctgtggccgtggtgaggaccgactccaggagtcccctttccctgtcccagccccatctgtgggttgcacttacaaacccccacccaccttcagaaatagtttttcaacatcaagactctctgtcgttgggattcatggcctattaaggaggtccaaggggtgagtcccaacccagccccagaatatatttgtttttgcacctgcttctgcctggagattgaggggtctgctgcaggcctcctccctgctgccccaaaggtatggggaagcaaccccagagcaggcagacatcagaggccagagtgcctagcccgacatgaagctggttccccaaccacagaaactttgtactagtgaaagaaaggggtccctggcctacgggctgaggctggtttctgctcgtgcttacagtgctgggtagtgttggccctaagagctgtagggtctcttcttcagggctgcatatctgagaagtggatgcccacatgccactggaagggaagtgggtgtccatgggccactgagcagtgagaggaaggcagtgcagagctggccagccctggaggtaggctgggaccaagctctgccttcacagtgcagtgaaggtacctagggctcttgggagctctgcggttgctaggggccctgacctggggtgtcatgaccgctgacaccactcagagctggaaccaagatctagatagtccgtagatagcacttaggacaagaatgtgcattgatggggtggtgatgaggtgccaggcactaggtagagcacctggtccacgtggattgtctcagggaagccttgaaaaccacggaggtggatgccaggaaagggcccatgtggcagaaggcaaagtacaggccaagaattgggggtgggggagatggcttccccactatgggatgacgaggcgagagggaagcccttgctgcctgccattcccagaccccagccctttgtgctcaccctggttccactggtctcaaaagtcacctgcctacaaatgtacaaaaggcgaaggttctgatggctgccttgctccttgctcccccaccccctgtgaggacttctctaggaagtccttcctgactacctgtgcccagagtgcccctacatgagactgtatgccctgctatcagatgccagatctatgtgtctgtctgtgtgtccatcccgccggccccccagactaacctccaggcatggactgaatctggttctcctcttgtacacccctcaaccctatgcagcctggagtgggcatcaataaaatgaactgtcgactgaaaaaaaaaa

SEQ ID NO: 16 - protein, 412 aa, Homo sapiens
MAENLKGCSVCCKSSWNQLQDLCRLAKLSCPALGISKRNLYDFEVEYLCDYKKIREQEYYLVKWRGYPDSESTWEPRQNLKCVRILKQFHKDLERELLRRHHRSKTPRHLDPSLANYLVQKAKQRRALRRWEQELNAKRSHLGRITVENEVDLDGPPRAFVYINEYRVGEGITLNQVAVGCECQDCLWAPTGGCCPGASLHKFAYNDQGQVRLRAGLPIYECNSRCRCGYDCPNRVVQKGIRYDLCIFRTDDGRGWGVRTLEKIRKNSFVMEYVGEIITSEEAERRGQIYDRQGATYLFDLDYVEDVYTVDAAYYGNISHFVNHSCDPNLQVYNVFIDNLDERLPRIAFFATRTIRAGEELTFDYNMQVDPVDMESTRMDSNFGLAGLPGSPKKRVRIECKCGTESCRKYLF

SEQ ID NO: 17 - protein, 90 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSP

SEQ ID NO: 18 - protein, 42 aa, Homo sapiens
MPIWKFPDEEGACQPCPINCTHSCVDKDDKGCPAEQRASPLT

SEQ ID NO: 19 - protein, 15 aa, synthetic construct
GGGGSGGGGSGGGGS

SEQ ID NO: 20 - protein, 20 aa, Homo sapiens
MYRMQLLSCIALSLALVTNS

SEQ ID NO: 21 - protein, 20 aa, Mus musculus
MYSMQLASCVTLTLVLLVNS

SEQ ID NO: 22 - protein, 20 aa, Homo sapiens
METPAQLLFLLLLWLPDTTG

SEQ ID NO: 23 - protein, 20 aa, Mus musculus
METDTLLLWVLLLWVPGSTG

SEQ ID NO: 24 - protein, 21 aa, Homo sapiens
MALPVTALLLPLALLLHAARP

SEQ ID NO: 25 - protein, 18 aa, Homo sapiens
MALPVTALLLPLALLLHA

SEQ ID NO: 26 - protein, 16 aa, synthetic construct
MKWVTFISLLFSSAYS

SEQ ID NO: 27 - protein, 30 aa, synthetic construct
MDSKGSSQKGSRLLLLLVVSNLLLCQGVVS

SEQ ID NO: 28 - DNA, 519 bp, Mus musculus
  source 1..519: mol_type = genomic DNA
gaggacctgcggaatgtcacgcccccgaaagtgtccctgtttgaaccaagtaaagccgagattgcgaataagcagaaggctactctggtttgcttggcacgaggattttttcctgaccatgtggaactgagctggtgggttaatgggaaagaagttcattcaggcgtatgtactgatccacaggcctacaaggaatcaaattactcttactgtctctcttcccgattgcgcgtttctgctacattttggcacaatcctcgaaatcatttccggtgccaagttcaattccacggtttgtcagaggaagacaaatggcccgagggcagtcctaagccagtaacgcagaatatatcagctgaggcgtggggcagagctgactgtggtataacttcagcgagctatcagcaaggggttctgagtgctactatcctctacgagatcctgctagggaaggccaccctgtatgctgtgctggtctctactctcgtggttatggcaatggtgaaacggaaaaacagc

SEQ ID NO: 29 - protein, 173 aa, Mus musculus
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 30 - DNA, 408 bp, Mus musculus
  source 1..408: mol_type = genomic DNA
atccagaaccctgagcccgctgtgtaccaattgaaagacccacggtcacaggacagtaccttgtgcctcttcacggactttgactctcagatcaacgttcctaagacgatggaaagtggcacatttatcacggataagtgtgtcttggatatgaaagctatggatagcaagtctaatggtgctatcgcatggtcaaaccagacttccttcacctgccaggatatttttaaggaaaccaatgcaacttacccatctagcgatgtaccatgcgatgccaccctgacggagaaaagctttgaaacagatatgaacctaaactttcaaaacctgctggtgattgtactgcgaatcctcctcctgaaagtggccgggtttaatctgctcatgacgctgcggctgtggtccagc

SEQ ID NO: 31 - protein, 136 aa, Mus musculus
IQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 32 - DNA, 930 bp, synthetic construct
  source 1..930: mol_type = other DNA
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttaaacggggcagaaagaaactcctgtatatattcaaacaaccatttatgagaccagtacaaactactcaagaggaagatggctgtagctgccgatttccagaagaagaagaaggaggatgtgaactgtga

SEQ ID NO: 33 - protein, 309 aa, synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFKRGRKKLLYIFKQPFMRPVQTTQEEDGCSCRFPEEEEGGCEL

SEQ ID NO: 34 - DNA, 360 bp, synthetic construct
  source 1..360: mol_type = other DNA
gaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtc

SEQ ID NO: 35 - DNA, 318 bp, synthetic construct
  source 1..318: mol_type = other DNA
gacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgattcacaggcagtggatctgggacagatttcactctcaccatcactaacgtgcagtctaaagacttggcagactatttctgtcaacaatataacaggtatccgtacacgtccggaggggggaccaagctggagatc

SEQ ID NO: 36 - protein, 120 aa, synthetic construct
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTV

SEQ ID NO: 37 - protein, 106 aa, synthetic construct
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEI

SEQ ID NO: 38 - DNA, 354 bp, synthetic construct
  source 1..354: mol_type = other DNA
gaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtc

SEQ ID NO: 39 - DNA, 318 bp, synthetic construct
  source 1..318: mol_type = other DNA
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatc

SEQ ID NO: 40 - protein, 118 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTV

SEQ ID NO: 41 - protein, 106 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEI

SEQ ID NO: 42 - DNA, 339 bp, synthetic construct
  source 1..339: mol_type = other DNA
gaggtacagctacaacaatcaggacccgaattgaagaaaccgggtacatccgtgcgcatcagctgtaagacctccggttatacctttaccgaatataccatccattgggttaaacagagccacggcaaatcgctggaatggattggtaacatcaacccgaacaacggtggcaccacgtacaatcagaagttcgaggacaaagccaccctgacggttgacaagagctcttccactgcgtatatggaactgcgtagcctgaccagcgaggacagcgcggtgtactactgcgcggctggttggaattttgattactggggtcaaggtacgaccttgaccgtc

SEQ ID NO: 43 - DNA, 318 bp, synthetic construct
  source 1..318: mol_type = other DNA
gatattgttatgacccagagccacaaattcatgagcacctctgtgggtgatcgcgtgtccatcatctgcaaggcgagccaggatgttggtactgccgtcgactggtaccagcaaaaaccgggtcaatccccaaaattgctgatttactgggccagcacgcgtcataccggggtgccggatcgttttaccggttctggcagcggcactgacttcacgctgaccattaccaatgttcagtcggaggacctggcagactacttctgccaacaatataacagctatccgctgacctttggtgcaggtacgatgttagatctt

SEQ ID NO: 44 - protein, 113 aa, synthetic construct
EVQLQQSGPELKKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTLTV

SEQ ID NO: 45 - protein, 106 aa, synthetic construct
DIVMTQSHKFMSTSVGDRVSIICKASQDVGTAVDWYQQKPGQSPKLLIYWASTRHTGVPDRFTGSGSGTDFTLTITNVQSEDLADYFCQQYNSYPLTFGAGTMLDL

SEQ ID NO: 46 - DNA, 420 bp, synthetic construct
  source 1..420: mol_type = other DNA
atccagaaccccgaccctgccgtctaccagctgagagattcaaaatcctccgacaaatccgtctgtctcttcaccgacttcgactcacagacaaatgtgtcccagtccaaggatagtgacgtgtacatcaccgacaaaactgtgctggacatgagatcaatggatttcaaatccaactccgccgtggcatggagtaacaaatccgatttcgcctgtgccaatgccttcaacaactccatcatccctgaggacaccttttttccatctcccgaatcatcttgtgacgtgaaactcgtcgagaaatcattcgaaaccgacaccaacctcaactttcagaatctgtccgtcatcggctttaggattctgctgctgaaagtggccggattcaatctgctcatgaccctgagactgtggtcatct

SEQ ID NO: 47 - DNA, 420 bp, synthetic construct
  source 1..420: mol_type = other DNA
attcagaatcctgatcctgccgtctaccagctgagagattcaaaatcctccgacaaatccgtctgtctcttcaccgacttcgactcacagacaaatgtgtcccagtccaaggatagtgacgtgtacatcaccgacaaaactgtgctggacatgagatcaatggatttcaaatccaactccgccgtggcatggagtaacaaatccgatttcgcctgtgccaatgccttcaacaactccatcatccctgaggacaccttttttccatctcccgaatcatcttgtgacgtgaaactcgtcgagaaatcattcgaaaccgacaccaacctcaactttcagaatctgtccgtcatcggctttaggattctgctgctgaaagtggccggattcaatctgctcatgaccctgagactgtggtcatct

SEQ ID NO: 48 - protein, 140 aa, synthetic construct; identical to SEQ ID NO: 4

SEQ ID NO: 49 - DNA, 540 bp, synthetic construct
  source 1..540: mol_type = other DNA
ctggaggatctgaaaaacgtgttccctcctgaagtggctgtctttgaaccatccgaggccgagatttcccatacccagaaagcaactctggtctgtctggccactggattctaccccgatcacgtggaactgtcttggtgggtgaacggcaaggaagtccattccggagtctctaccgaccctcagcccctcaaggagcagcctgctctcaacgattctcggtactgcctgtcatctcgactgagagtgtctgccaccttctggcagaaccctagaaaccactttcggtgtcaggtccagttttacggcctgagcgagaacgatgagtggacacaggatagagccaaacctgtgacacagattgtgagcgctgaggcttggggacgagccgattgtggcttcacatccgagtcttaccagcagggagtgctgtctgctacaatcctctacgaaattctcctggggaaggccaccctgtacgctgtcctcgtgtctgctctggtgctcatggctatggtcaaacgaaaggactctagaggc

SEQ ID NO: 50 - protein, 180 aa, synthetic construct
LEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 51 - DNA, 66 bp, synthetic construct
  source 1..66: mol_type = other DNA
ggctctggcgctaccaatttttccctcctcaaacaggctggagatgtcgaagagaaccccggacct

SEQ ID NO: 52 - DNA, 63 bp, synthetic construct
  source 1..63: mol_type = other DNA
atggctctcccagtgactgccctactgcttcccctagcgcttctcctgcatgcagccaggccg

SEQ ID NO: 53 - DNA, 801 bp, synthetic construct
  source 1..801: mol_type = other DNA
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgcttt

SEQ ID NO: 54 - protein, 267 aa, synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCF

SEQ ID NO: 55 - DNA, 925 bp, synthetic construct
  source 1..925: mol_type = other DNA
cgaggcagggctttggctactggagatcgtaggttcgaatcccgtctgggaagttcaacttgtgcacctgtaaaagaagctggcattattggcttgtactcaagggctggcacagagtgtgtcggggtgcggacgccccagccacgcccatcatccgtgcggaagatgcagaggtcatatcggatacccttctgtaccacacgatttgggcagtcatagccgcagcggcagcgggagttgcactcgtagatgggcagcccggctcgaagccgcacctggccctggtcattgtaggcaaacttgtgcagtgacgcccccgggcagcagcctccagtgggtgcccacagacagtcctggcactcgcagcccacagccacctggttgagggtgatgccctcaccaacacggtactcattgatgtacacgaaggcccgcggagggccgtccaggtccacctcattctctacagtgatgcgtcccagatggctgcgcttggcattgagctcctgctcccagcgacggagcgcccgcctctgcttggccttctgcaccaggtagttggccaagcttgggtccaggtgccggggggtctttgaccggtggtgccgccggagcagctccctttctaagtccttgtggaactgcttgaggatacgcacacacttgagattctgccgtggctcccaggtgctctctgagtctggatatccacgccatttcaccagggtcaaaggagaaaattccctttggaaacagatgtgggcagttggggacaagagggcaggacactaacttccttgtgacctgtcccctcccagagcatggtcaccccagactcacgcggatcttcttgtaatcgcacaggtactcgacttcaaagtcatagaggttcctcttagagataccgagggcagggcaggagagc

SEQ ID NO: 56 - RNA, 925 nt, synthetic construct
  source 1..925: mol_type = other RNA
cgaggcagggctttggctactggagatcgtaggttcgaatcccgtctgggaagttcaacttgtgcacctgtaaaagaagctggcattattggcttgtactcaagggctggcacagagtgtgtcggggtgcggacgccccagccacgcccatcatccgtgcggaagatgcagaggtcatatcggatacccttctgtaccacacgatttgggcagtcatagccgcagcggcagcgggagttgcactcgtagatgggcagcccggctcgaagccgcacctggccctggtcattgtaggcaaacttgtgcagtgacgcccccgggcagcagcctccagtgggtgcccacagacagtcctggcactcgcagcccacagccacctggttgagggtgatgccctcaccaacacggtactcattgatgtacacgaaggcccgcggagggccgtccaggtccacctcattctctacagtgatgcgtcccagatggctgcgcttggcattgagctcctgctcccagcgacggagcgcccgcctctgcttggccttctgcaccaggtagttggccaagcttgggtccaggtgccggggggtctttgaccggtggtgccgccggagcagctccctttctaagtccttgtggaactgcttgaggatacgcacacacttgagattctgccgtggctcccaggtgctctctgagtctggatatccacgccatttcaccagggtcaaaggagaaaattccctttggaaacagatgtgggcagttggggacaagagggcaggacactaacttccttgtgacctgtcccctcccagagcatggtcaccccagactcacgcggatcttcttgtaatcgcacaggtactcgacttcaaagtcatagaggttcctcttagagataccgagggcagggcaggagagc

(19) United States
(12) Patent Application Publication
(10) Pub. No.: US 2026/0116947 A1
(43) Pub. Date: Apr. 30, 2026

(54) IMMUNE CELLS WITH INACTIVATED SUV39H1 AND MODIFIED TCR

(71) Applicant: MNEMO THERAPEUTICS, Paris (FR)

(72) Inventors: Francois GAUDET, New York, NY (US); Michael SAITAKIS, Paris (FR); Priyanka NAIR-GUPTA, Paris (FR); Madhavi Latha SOMARAJU, Paris (FR); Theodoros GIAVRIDIS, Paris (FR); Saswati PANDA, Paris (FR); Kevin JHUN, Paris (FR); Kayla VAN BUREN, Paris (FR); Yao WEI, Paris (FR); Armelle BOHINEUST, Paris (FR); Angela YINGZHE LI, Paris (FR)

(21) Appl. No.: 18/724,902

(22) PCT Filed: Dec. 28, 2022
(86) PCT No.: PCT/EP2022/087979

§ 371 Date: Jun. 27, 2024

Related U.S. Application Data
(60) Provisional application No. 63/294,201, filed on Dec. 28, 2021.

(51) Int. Cl. C07K 14/725; A61K 39/395; A61K 40/11; A61K 40/32; A61K 40/42; A61P 35/00; C12N 5/0783

(52) U.S. Cl. CPC C07K 14/7051; A61K 39/39558; A61K 40/11; A61K 40/32; A61K 40/4211; A61P 35/00; C12N 5/0636; C12N 2501/72; C12N 2510/00

(57) ABSTRACT

The present disclosure relates to an improved immune cell expressing an antigen-specific receptor such as a CAR or TCR, in which SUV39H1 is inactivated, optionally combined with disruption of the TRAC locus and/or deletion of one or more ITAMs. The disclosure also provides compositions comprising such cells, methods of producing such cells, and uses of such cells in adoptive cell therapy, e.g. in cancer or inflammatory diseases.

### FIELD OF THE DISCLOSURE

The present disclosure relates to the field of adoptive cell therapy. The present disclosure provides immune cells that express modified TCR and in which SUV39H1 has been inactivated, which exhibit enhanced properties.

### INTRODUCTION

Adoptive T cell therapy (ATCT) using T cells armed with recombinant T Cell Receptor (TCR) and Chimeric Antigen Receptor (CAR) technologies is emerging as a powerful cancer therapy alternative (Lim W A & June C H. 2018. Cell 168(4):724-740). Efficient engraftment, long-term persistence and reduced exhaustion of the therapeutic T cells correlates with positive therapeutic outcomes. Additionally, the increased persistence of adoptively transferred cells appears to be dependent upon the acquisition of central memory T cell (TCM) populations (Powell D J et al., Blood. 2005; 105(1):241-50; Huang J, Khong H T et al. J Immunother. 2005; 28:258-267).

A major obstacle for the successful cell-based therapy of solid tumors is the exhaustion of activated T cells, which decreases their ability to proliferate and destroy target cells. PD-1 blockade can restore T cell function at an early stage but the rescue may be incomplete or transient (Sen D R, et al. 2016. Science 354(6316):1165-1169; Pauken K E, et al. 2016. Science 354(6316):1160-1165). Moreover, the immunosuppressive microenvironment in the tumor mediates T cell exhaustion (Joyce J A, Fearon D T. 2015. Science 348(6230):74-80).

There remains a need in the art for modified or engineered T cells with improved properties for adoptive cell therapy.

### SUMMARY OF THE DISCLOSURE

The disclosure provides immune cells, particularly T-cells or NK cells or progenitors thereof, that express one or more modified antigen-specific receptors and in which SUV39H1 has been inactivated, as well as compositions, kits and methods of manufacture and methods of use relating to such immune cells. The modified immune cells of the disclosure express an antigen-specific receptor that comprises a heterologous antigen-binding domain that specifically binds a target antigen. The antigen-specific receptor may comprise an extracellular domain that comprises antigen-binding fragments or CDRs of an antibody, preferably all three CDRs of a heavy chain variable region (VH) and/or a light chain variable region (VL). The antigen-specific receptor may be a chimeric antigen receptor (CAR) or a heterologous TCR, e.g. a modified TCR.

The modified TCR may comprise (a) an extracellular domain that comprises antigen-binding fragments or CDRs of an antibody, preferably all three CDRs of a heavy chain variable region (VH) and/or a light chain variable region (VL), and (b) a native or variant constant region of an alpha, beta, gamma or delta chain. For example, the modified TCR may comprise one or more heterologous polypeptides, for example, (a) VH of an antibody or a fragment or variant having at least 90% sequence identity thereto, fused to TRAC (SEQ ID NO: 4), or a fragment or variant of TRAC (SEQ ID NO: 4) having at least 90% sequence identity thereto or fused to TRBC1 (SEQ ID NO: 5) or TRBC2 (SEQ ID NO: 6), or a fragment or variant of TRBC1 (SEQ ID NO: 5) or TRBC2 (SEQ ID NO: 6) having at least 90% sequence identity thereto, and (b) a VL of an antibody or a fragment or variant having at least 90% sequence identity thereto fused to TRAC (SEQ ID NO: 4), or a fragment or variant of TRAC (SEQ ID NO: 4) having at least 90% sequence identity thereto or fused to TRBC1 (SEQ ID NO: 5) or TRBC2 (SEQ ID NO: 6), or a fragment or variant of TRBC1 (SEQ ID NO: 5) or TRBC2 (SEQ ID NO: 6) having at least 90% sequence identity thereto. See FIG. 1A. The modified TCR capable of associating with (and consequently activating) a CD3zeta polypeptide that can be a native or variant e.g. a modified CD3zeta polypeptide (SEQ ID NO: 7) in which one or two of the ITAM domains (e.g. ITAM2 and ITAM3) have been deleted. In some embodiments, the modified TCR may optionally further comprise a native or variant CD3zeta polypeptide, e.g. a modified CD3zeta polypeptide (SEQ ID NO: 7) in which one or two of the ITAM domains (e.g. ITAM2 and ITAM3) have been deleted. See FIG. 1B.

Recombinant HLA-independent (or non-HLA restricted) modified TCR (referred to as “HI-TCRs”) that bind to an antigen of interest in an HLA-independent manner are described in International Application No. WO 2019/157454. Such HI-TCRs comprise an antigen binding chain that comprises: (a) a heterologous antigen-binding domain that binds to an antigen in an HLA-independent manner, for example, an antigen-binding fragment of an immunoglobulin variable region; and (b) a constant domain that is capable of associating with (and consequently activating) a CD3zeta polypeptide. Preferably, the antigen-binding domain or fragment thereof comprises: (i) a heavy chain variable region (VH) of an antibody and/or (ii) a light chain variable region (VL) of an antibody. The constant domain of the TCR is, for example, a native or modified TRAC polypeptide (SEQ ID NO: 4 or variant thereof), or a native or modified TRBC polypeptide (SEQ ID NO: 5 or 6 or variant thereof). The constant domain of the TCR is, for example, a native TCR constant domain (alpha or beta) or fragment thereof. Unlike chimeric antigen receptors, which typically themselves comprise an intracellular signaling domain, the HI-TCR does not directly produce an activating signal; instead, the antigen-binding chain associates with and consequently activates a CD3zeta polypeptide (SEQ ID NO: 7). The immune cells comprising the recombinant TCR provide superior activity when the antigen has a low density on the cell surface of less than about 10,000 molecules per cell, e.g. less than about 5,000, 4,000, 3,000, 2,000, 1,000, 500, 250 or 100 molecules per cell.

The CD3zeta polypeptide optionally comprises an intracellular domain of a co-stimulatory molecule or a fragment thereof. Alternatively, the antigen binding domain optionally comprises a co-stimulatory domain that is capable of stimulating an immunoresponsive cell upon the binding of the antigen binding chain to the antigen. Example co-stimulatory domains include stimulatory domains, or fragments or variants thereof, from CD28 (SEQ ID NO: 8-9), 4-1BB (CD137) (SEQ ID NO: 10-11), ICOS (SEQ ID NO: 12), CD27, OX 40 (CD134) (SEQ ID NO: 13), DAP10, DAP12, 2B4, CD40, FCER1G or GITR (AITR). For T cells, CD28, CD27, 4-1BB (CD137), ICOS may be preferred. For NK cells, DAP10, DAP12, 2B4 may be preferred. Combinations of two co-stimulatory domains are contemplated, e.g. CD28 and 4-1BB, or CD28 and OX40.

The foregoing modified immune cell expressing an antigen-specific receptor, e.g. modified TCR, preferably comprises one or more further features as described herein: inactivation (e.g. mutation or inhibition) of the SUV39H1 gene, and/or inactivation of one or two ITAM domains of the CD3zeta intracellular signaling region of the antigen-specific receptor, and/or inactivation of one or both endogenous TCR chains (e.g. deletion or disruption of endogenous TCR-alpha and/or TCR-beta) and/or addition of a co-stimulatory receptor, or combinations of one, two, three or all of such features.

In one aspect, the SUV39H1 gene of the modified immune cell (SEQ ID NO: 15) is inactivated. In some embodiments, the modified immune cell may comprise one or more mutations (insertion, substitution, deletion) that results in a deleted or non-functional SUV39H1 protein. In other embodiments, the modified immune cell is contacted with an agent that inhibits SUV39H1 activity by at least 50%, preferably 60%, 70%, 80%, 90% or more, and is cultured under such conditions of SUV39H1 inhibition for a time period sufficient to produce enhanced properties. The agent that inhibits SUV39H1 may be expressed by the cell or delivered to the cell by known transfection methods. Immune cells in which SUV39H1 has been inactivated or inhibited exhibit an enhanced central memory phenotype, enhanced survival and persistence after adoptive transfer, and reduced exhaustion. In particular, such cells accumulate and re-program with increased efficiency into long-lived central memory cells. Such cells are more efficient at inducing tumor cell rejection and display enhanced efficacy for treating cancer.

In another aspect, the antigen-specific receptor (e.g. modified TCR) comprises a modified CD3 with a single active ITAM domain, and optionally the CD3 may further comprise one or more or two or more co-stimulatory domains. For example, the antigen-specific receptor comprises a modified CD3zeta intracellular signaling domain in which ITAM2 and ITAM3 have been inactivated. In some embodiments, ITAM1 and ITAM2 have been inactivated, or ITAM2 and ITAM3 have been inactivated. In some embodiments, a modified CD3zeta polypeptide of a modified TCR retains only ITAM1 and the remaining CD3zeta domain is deleted (residues 90-164 of SEQ ID NO: 7). See FIG. 1B. As another example, ITAM1 is substituted with the amino acid sequence of either ITAM 2 or ITAM3, and the remaining CD3zeta domain is deleted (residues 90-164 of SEQ ID NO: 7).

In yet a further aspect, at least one T cell receptor (TCR) constant region gene of the foregoing modified immune cell is modified by the insertion of a nucleic acid sequence encoding the antigen-specific receptor or the antigen-binding domain. The TCR constant region is a TCR alpha constant region (TRAC) and/or a TCR beta constant region (TRBC). According to this aspect, the insertion of the nucleic acid sequence can disrupt or abolish the endogenous expression of a TCR comprising a native TCR alpha chain and/or a native TCR beta chain. For example, the nucleic acid encoding the antigen-specific receptor may be heterologous to the immune cell and operatively linked to an endogenous promoter of the T-cell receptor such that its expression is under control of the endogenous promoter. In some embodiments, a nucleic acid encoding a CAR is operatively linked to an endogenous TRAC promoter. The insertion of the nucleic acid sequence may reduce endogenous TCR expression by at least about 75%, 80%, 85%, 90% or 95%.

In a related aspect, the antigen-specific receptor is a modified TCR comprising a heterologous antigen-binding domain and a native TCR constant domain (alpha or beta) or fragment thereof, and the antigen-specific receptor (modified TCR) is capable of activating a CD3zeta polypeptide. In some embodiments, the nucleic acid encoding the heterologous antigen-binding domain can be inserted into the endogenous TRAC locus and/or TRBC locus of the immune cell. For example, the nucleic acid encoding the antigen-specific receptor may be heterologous to the immune cell and operatively linked to an endogenous promoter of the T-cell receptor such that its expression is under control of the endogenous promoter. The insertion of the nucleic acid sequence can thus disrupt or abolish the endogenous expression of a TCR comprising a native TCR alpha chain and/or a native TCR beta chain. The insertion of the nucleic acid sequence may reduce endogenous TCR expression by at least about 75%, 80%, 85%, 90% or 95%. According to this aspect, a nucleic acid encoding an antigen binding domain can be inserted in the TRBC locus to produce a fusion polypeptide comprising the antigen binding domain, or fragment thereof, fused to a TCR beta constant region, or fragment thereof. In addition, or alternatively, an antigen binding domain (that can be the same as the antigen binding domain fused to the TRBC locus or different), can be inserted in the TRAC locus to produce a fusion polypeptide comprising it, fused to a TCR alpha constant region, or fragment thereof. More particularly, a nucleic acid encoding a heavy chain variable region (VH) of an antibody, or a fragment thereof, can be inserted in the TRBC locus to produce a fusion polypeptide comprising the VH, or fragment thereof, fused to a TCR beta constant region, or fragment thereof. In addition, a nucleic acid encoding a light chain variable region (VL) of an antibody, or a fragment thereof, can be inserted in the TRAC locus to produce a fusion polypeptide comprising the VL, or fragment thereof, fused to a TCR alpha constant region, or fragment thereof. Alternatively, the VH or fragment thereof can be fused to the TCR alpha constant region, or fragment thereof, and the VL or fragment thereof can be fused to the TCR beta constant region, or fragment thereof. In some embodiments, a single nucleic acid encoding the modified TCR-beta chain and modified TCR-alpha chain, is operatively linked to an endogenous TRAC promoter. See FIG. 2A. In some embodiments, the modified TCR-beta chain and TCR-alpha chain are separated by a self-cleavable linker, such as peptide 2A.

In another aspect, the immune cell comprising the modified TCR also comprises a co-stimulatory receptor. In other aspects, the immune cell comprising the modified TCR does not comprise a co-stimulatory receptor, e.g. does not comprise a CD80/4-1BB chimeric receptor as described in Int'l. Pat. Pub. No. WO 2021/016174. Such co-stimulatory receptors include chimeric receptors comprising a co-stimulatory ligand fused to at least one or at least two co-stimulatory molecule(s). Co-stimulatory ligands include CD80, CD86, 41 BBL, CD275, CD40L, OX40L or any combination thereof. In some embodiments, the co-stimulatory ligand is CD80 or 4-1BBL. Example co-stimulatory molecules are CD28, 4-1BB, OX40, ICOS, DAP-10, CD27, CD40, NKG2D, CD2, or any combination thereof. In some embodiments, the co-stimulatory receptor comprises (a) an extracellular and transmembrane domain of CD86, 41 BBL, CD275, CD40L, OX40L, PD-1, TIGIT, 2B4, or NRP1, or fragment or variant thereof, and (b) an intracellular co-stimulatory molecule of CD28, 4-1BB, OX40, ICOS, CD27, CD40, or CD2, or fragment or variant thereof. In some embodiments, the chimeric receptor comprises a first co-stimulatory molecule that is 4-1BB and a second co-stimulatory molecule that is CD28. A preferred chimeric receptor comprises a CD80 co-stimulatory ligand (SEQ ID NO: 14) and a 4-1BB co-stimulatory molecule (SEQ ID NO: 10). Co-stimulatory receptors are described in Int'l. Pat. Pub. No. WO 2021/016174.

The modified immune cells disclosed herein may comprise combinations of two or more of the foregoing aspects.

For example, the modified immune cell is an immune cell wherein (a) the SUV39H1 gene is inactivated or inhibited, and (b) the antigen-specific receptor is a modified TCRαβ comprising a heterologous antigen-binding domain and at least one native TCR constant domain or fragment thereof, and the TCRαβ is capable of activating a CD3zeta polypeptide. As another example, the modified immune cell further comprises (c) CD3zeta intracellular signaling domain with a single active ITAM domain, e.g. in which ITAM2 and ITAM3 have been inactivated and/or (d) a co-stimulatory receptor, e.g. CD80 extracellular domain (SEQ ID NO: 14) linked to a 4-1BB intracellular co-stimulatory domain (SEQ ID NO: 10).

As another example, the modified immune cell is an immune cell (a) wherein the SUV39H1 gene is inactivated and (b) the immune cell expresses a chimeric antigen receptor (CAR) comprising: (i) an extracellular antigen-binding domain, (ii) a transmembrane domain, (iii) optionally one or more costimulatory domains, and (iv) an intracellular signaling domain comprising a modified CD3zeta intracellular signaling domain with a single active ITAM domain, e.g. in which ITAM2 and ITAM3 have been inactivated.

In any of the aspects or embodiments herein, the antigen-binding domain may bind the target antigen with a binding affinity Kd of 10⁻⁷ M or less, or 10⁻⁸ M or less, or 10⁻⁹ M or less (smaller numbers indicating higher affinity).

In any of the aspects described herein, the modified immune cell may be a T cell, a CD4+ T cell, a CD8+ T cell, a CD4+ and CD8+ T cell, a NK cell, a Treg cell, a Tm cell, a memory stem T cell (TSCM), a TCM cell, a TEM cell, a T cell progenitor, an NK cell progenitor, a pluripotent stem cell, an induced pluripotent stem cell (iPSC), a hematopoietic stem cell (HSC), an adipose derived stem cell (ADSC), or a pluripotent stem cell of myeloid or lymphoid lineage.

In any of the aspects described herein, the antigen-specific receptor is a CAR comprising: (a) an extracellular antigen-binding domain; (b) a transmembrane domain, (c) optionally one or more costimulatory domains, and (d) an intracellular signaling domain. The extracellular antigen-binding domain may be a scFv, optionally an scFv that specifically binds a target antigen as disclosed herein.

In any of the aspects herein, the antigen-binding domain binds any one or more of the following antigens: ADGRE2, alphafetoprotein (AFP), BCMA, carcinoembryonic antigen (CEA), CAIX, CCR1, a cyclin, such as cyclin AI (CCNA1) or cyclin (D1), CEA, CE7, CD7, CD8, CD10, CD19, CD20, CD22, CD23, CD24, CD30, CD70, CLL1, CD33, CD34, CD38, CD41, CD44, CD44V6, CD49f, CD56, CD74, CD99, CD123, CD133, CD138, CS-1, Claudin 18.2, c-Met, cytochrome P450 1 B1 (CYP1 B), EGF1R, EGFR, EGFR-VIII, EGP-2, EGP-4, EGP-40, EpCAM, EPHa2, ephrinB2, ERBB, Erb-B2, Erb-B3, Erb-B4, estrogen receptor, FBP, FcRH5, Fetal acetylcholine receptor, folate receptor-a, GD2, GD3, GP100, gplOO, HMW-MAA, HER2/neu, hepatitis B surface antigen, human telomerase reverse transcriptase (hTERT), IL-22R-alpha, IL-13R-alpha2, K-light chain, KDR, Lewis Y, L1-cell adhesion molecule, LILRB2, LILRB4, LI-CAM, livin, MAGE-A1, MAGE-A3, MART-1, mesothelin, mouse double minute 2 homolog (MDM2), mucin 16 (MUC16), MUC1, NKCS1, NKG2D Ligands, NY-ESO-1, oncofetal antigen (h5T4), orphan tyrosine kinase receptor (ROR1), p53, pHER95, p95HER2, PRAME, progesterone receptor, prostate-specific membrane antigen (PSMA), prostate specific antigen, Proteinase3 (PR1), PSCA, Survivin, TAG-72, tEGFR, Tyrosinase, VEGF-R2, Wilms' Tumor gene 1 (WT-1). In some embodiments, the antigen may be any of the tumor neoantigenic peptides disclosed in any one of Int'l Pat. Pub. No. WO 2021/043804, WO 2022/189620, WO 2022/189626, and WO 2022189638 incorporated by reference herein in its entirety. The antigen may alternatively be an antigen associated with an infectious disease, an autoimmune disease, or an inflammatory disease.

In any of these embodiments, the antigen-specific receptor may be a bispecific antigen-specific receptor that binds both (a) a first antigen (e.g. a cancer antigen) and (b) a T cell activation antigen, e.g. CD3.

In any of these embodiments, the immune cell further secretes a non-membrane-bound (soluble) bispecific antibody, e.g. a BiTE (bispecific T cell engager soluble antibody), that binds to both the target and a T cell activation antigen, e.g. CD3 epsilon or the constant chain (alpha or beta) of a TCR.

In any of these embodiments, the immune cell may further comprise a second antigen-specific receptor, optionally a modified TCR or CAR, that specifically binds to a second antigen. For example, the immune cell may comprise two TCRs, a first TCR that binds a first antigen and a second TCR that binds a second antigen, or a TCR that binds a first antigen and a CAR that binds a second antigen.

In any of these embodiments, inactivation of SUV39H1 reduces SUV39H1 gene expression of SUV39H1 protein activity by at least about 50%, 60%, 70%, 75%, 80%, 85%, 90% or 95%.

In any of these embodiments, the immune cell may be autologous or allogeneic. In any of these embodiments, the immune cell is modified to reduce immunogenicity, e.g. the HLA-A locus is inactivated and/or beta-2-microglobulin is inactivated. In some embodiments, HLA class I expression is reduced by at least about 75%, 80%, 85%, 90% or 95%.

The disclosure also provides, in another aspect, methods of making the modified immune cells (e.g. T cell, NK cell, or progenitors thereof) of the disclosure, and vectors for making the modified immune cells. Such methods include introducing into the immune cell (including progenitors) via homologous recombination a nucleic acid (e.g., vector) encoding one or more components of a modified TCR as described herein, e.g. (a) modified TCR beta chain comprising a VH or fragment or variant thereof, (b) a modified TCR alpha chain comprising a VL or fragment or variant thereof, and optionally (c) a modified CD zeta chain in which at least two ITAMs have been deleted, or any of the alternative embodiments described herein. Such methods also include introducing into the immune cell (including progenitors), a nucleic acid encoding a co-stimulatory receptor as described herein. Such methods may further include inactivating the SUV39H1 gene according to any of the methods disclosed herein, e.g., by introducing a mutation or knocking out most or all of the gene, or by contacting the cell with a SUV39H1 inhibitor that reduces SUV39H1 gene expression or SUV39H1 protein activity.

The disclosure also provides, in another aspect, a sterile pharmaceutical composition comprising any of the foregoing modified immune cells. The disclosure also provides a kit comprising any of the foregoing modified immune cells and a delivery device or container.

The disclosure further provides a method of using the foregoing modified immune cell or pharmaceutical composition or kit to treat a patient suffering from or at risk of disease associated with the antigen, optionally cancer, by administering a therapeutically effective amount of said immune cell or pharmaceutical composition to the patient. In some embodiments, the immune cell is a T-cell or NK cell and a dose of less than about 5×10⁷ cells, optionally about 10⁵ to about 10⁷ cells, is administered to the patient. The method may further comprise administering to the patient a second therapeutic agent, optionally one or more cancer chemotherapeutic agents, cytotoxic agents, hormones, anti-angiogens, radiolabelled compounds, immunotherapy, surgery, cryotherapy, and/or radiotherapy, is administered to the patient. The second therapeutic agent may be an immune checkpoint modulator. Examples of an immune checkpoint modulator include an antibody that specifically binds to, or an inhibitor of, PD1, PDL1, CTLA4, LAG3, BTLA, OX2R, TIM-3, TIGIT, LAIR-1, PGE2 receptor, EP2/4 adenosine receptor, or A2AR.

### BRIEF DESCRIPTION OF THE DRAWINGS

FIGS. 1A and 1B depict a schematic of a modified TCR of the present disclosure.

FIG. 2A depicts a schematic of cassette(s) introduced to an immune cell to produce the modified immune cells described herein. FIG. 2B depicts a schematic of the expressed TCR and co-stimulatory receptor.

FIG. 3 illustrates that transduced T-cells show LNGFR (for gRV-66HIT)/CD80 (for gRV-66HITBooster) expression and bind to human MSLN-Fc fusion protein. (A) Representative LNGFR/CD80-PE, huMSLN Fc fusion protein-FITC flow plots of scr/scr, SUV KO/Scr, SUV KO/TRAC KO T-lymphocytes (donor 1), 5 days after transduction with gRV-66HIT and gRV-66HIT Booster (CD80) Galv9 viral supernatants. Untransduced T-cells (UT) were used as controls. The plots shown here were pre-gated on FSC-A/SSC-A>Singlets>Live cells>CD4+CD8+. (B) Bar graph showing mean±S.D. of % LNGFR/CD80+ MSLN+ cells from 2 different donors.

FIG. 4 illustrates that transduced T-cells show CD3 reconstitution with surface binding to recombinant mesothelin. (A) Representative CD3-pacific blue, huMSLN Fc fusion protein-FITC flow plots of scr/scr, SUV KO/Scr, SUV KO/TRAC KO T-lymphocytes (donor 1), 5 days after transduction with Galv9 gRV-66HIT and gRV-66HIT Booster (CD80) viral supernatants. Untransduced T-cells (UT) were used as controls. The plots shown here were pre-gated on FSC-A/SSC-A>Singlets>Live cells>CD4+CD8+. (B) Bar graph showing mean±S.D. of % CD3med+MSLN+ cells from 2 different donors.

FIG. 5 illustrates efficient knockdown of SUV39H1 by electroporation with SUV39H1 guide RNA. (A) Representative Western blots for SUV39H1 and GAPDH in scramble versus SUV39H1 knockdown samples of untransduced, gRV-66HIT and gRV-66HITBooster T-cells from donor 1. (B) Bar graph showing mean±S.D. of SUV39H1 expression from two different donors. GAPDH was used as loading control.

FIG. 6 illustrates that SUV39H1 deficient gRV-66HIT cells show increased cytotoxicity of OVCAR-3 tumor cells (at lower E:T ratios) and is comparable to the activity observed with gRV-66HITBooster (CD80) cells. Luciferase expressing OVCAR-3 cells were co-cultured with gRV-66HIT or gRV-66HITBooster (scr/TRAC KO or SUV KO/TRAC KO) T-cells at the indicated E:T ratios for 48 hr and cell lysis was measured using luminescence based Bright-Glo™ luciferase assay. Untransduced (UT) T-cells (scr/TRAC KO or SUV KO/TRAC KO) were used as controls. Line graphs showing mean±S.D. of % cytotoxicity from 2 different donors in duplicates. P values were calculated by two-way ANOVA test. ***p<0.0002, ****p<0.0001.

FIG. 7 illustrates that SUV39H1 depleted gRV-66HIT cells show increased T-cell expansion, persistence, memory and target killing capacity on repeated antigen exposure with NOMO-1 target lines. Scramble and SUV KO gRV-66HIT cells were co-cultured with NOMO-1 target line at E:T of 1:2 (1e⁵ HIT T-cells and 2e⁵ target cells on day 0) in 24-well G-REX plate and flow cytometry was done every 4th day followed by in order to estimate HIT T-cell and target cell numbers. Target cells were re-introduced post analysis to maintain E:T of 1:2. Bar graphs showing (A) total gRV-66HIT cells and (B) total target cells on day 3, 7 & 10. Donor cells: 110046475. Line plots showing (C) % CD45RO⁺ CD27⁺ and (D) % CD45RO⁺ CCR7⁺ transduced HIT T-cells. (E) CD8/CD4 ratio of transduced HiT cells.

FIG. 8 illustrates that SUV39H1 inactivated gRV-66HIT Booster (CD80 booster) cells show increased T-cell expansion, persistence, memory and target killing capacity on repeated antigen exposure with NOMO-1 target lines. Scramble and SUV KO gRV-66HITBooster cells were co-cultured with NOMO-1 target line at E:T of 1:2 (1e5 HIT T-cells and 2e5 target cells on day 0) in 24-well G-REX plate and flow cytometry was done every 4th day followed by in order to estimate HIT T-cell and target cell numbers. Target cells were re-introduced post analysis to maintain E:T of 1:2. Bar graphs showing (A) total gRV-66HITBooster cells and (B) total target cells on day 3, 7 & 10. Donor 3. Line plots showing (C) % CD45RO+CD27+ and (D) % CD45RO+ CCR7+ transduced HIT T-cells. (E) CD8/CD4 ratio of transduced HiTBooster (CD80) cells.”

FIG. 9 illustrates that SUV39H1 inactivated gRV-66HIT Booster (CD80) cells show increased T-cell expansion, persistence, memory and target killing capacity on repeated antigen exposure with NOMO-1 target lines. Scramble and SUV KO gRV-66HITBooster cells were co-cultured with NOMO-1 target line at E:T of 1:2 (1e5 HIT T-cells and 2e5 target cells on day 0) in 24-well G-REX plate and flow cytometry was done every 4th day followed by in order to estimate HIT T-cell and target cell numbers. Target cells were re-introduced post analysis to maintain E:T of 1:2. Bar graphs showing (A) total gRV-66HITBooster cells at day 8, 19, 22 & 26 and (B) total target cells at day 8, 19, 22, 26 & 36. Donor 3. Line plots showing (C) % CD45RO+CD27+ and (D) % CD45RO+ CCR7+ transduced HIT T-cells. (E) CD8/CD4 ratio of transduced HiTBooster cells.

FIG. 10 illustrates that SUV39H1 depleted gRV-66HIT and gRV-66HIT Booster (CD80) cells show increased T-cell expansion, persistence and memory on repeated antigen exposure with NOMO-1 target lines. Scramble and SUV KO gRV-66HIT and gRV-66HIT Booster cells were co-cultured with NOMO-1 target line at E:T of 1:2 (1e⁵ HIT T-cells and 2e⁵ target cells on day 0 in 24-well G-REX plate and flow cytometry was done every 4th day in order to estimate HIT T-cell and target cell numbers. Target cells were re-introduced post analysis to maintain E:T of 1:2. Bar graphs showing (A) total gRV-66HIT cells and (B) total gRV-66HIT Booster T-cells on day 7, 28, 34 & 42. Line plots showing % CD45RO⁺ CCR7⁺ transduced (C) gRV-66 HIT and (D) gRV-66HIT Booster T-cells on day 0, 7, 28, 34 & 42. Donor cells: 888689798.

FIG. 11 shows a schematic representation of gammaretroviral (gRV) HIT constructs illustrated herein. Construct A includes the transduction marker LNGFR. Construct B includes a booster molecule.

FIG. 12 shows Western blot of SUV39H1 to determine SUV-KO on representative samples of FMC63-HiT+Booster and mJ591-HiT+Booster (CD80_4-1BB) engineered T cells. (A) Protein quantification of samples against standard curve. All samples shown in figure were within range and loaded equally. (B) Ponceau staining of membrane after Western Blot process. (C) Western Blot confirms knockout of SUV39H1 band (lower band, highlighted in black box) at 48 kDa. Nonspecific band above has been verified to be unchanged with KO of SUV39H1, when comparing WT to KO samples. There are 2 ladder lanes on the left-hand side and 1 ladder lane on the right. Data representative of N=2 donors and error bars are SEM.

FIG. 13 shows representative dot plots of successful transduction of T cells with gRV HiT constructs and successful TRAC-KO based on loss of CD3 expression. (A) Dot plots show gRV-FMC63-HiT (left) and gRV-FMC63-HiT+Booster cells when transduced with their respective virus based on either LNGFR or booster expression. (B) Successful TRAC-KO can be observed with the loss of CD3 when compared to the expression markers (similar CD3+ population reduction as in FIG. 4). Data representative of N=2 donors and error bars are SEM.

FIG. 14 shows representative dot plots show specific binding of recombinant CD19 protein to FMC63-HiT T cells. (A) TRACKO/Scrambled (SCR) and (B) TRACKO/SUVKO samples incubated with recombinant avi-tagged CD19 protein and stained with streptavidin-A647. Both gRV-FMC63-HiT (middle) and gRV-FMC63-HiT+Booster (right) show similar binding to the recombinant CD19 protein in combination with their respective expression marker (LNGFR and Booster); the negative control (left) was stained only with streptavidin-A647 and no recombinant protein. C) Percentage of CD19-interacting cells. Data representative of N=2 donors and error bars are SEM.

FIG. 15 shows representative dot plots of successful transduction of T cells with gRV HiT constructs and successful TRAC-KO based on loss of CD3 expression. (A) Dot plots show gRV-mJ591-HiT (left) and gRV-mJ591-HiT+Booster cells when transduced with their respective virus based on either LNGFR or booster expression. (B) Successful TRAC-KO can be observed with the loss of CD3 when compared to the expression markers (similar CD3+ population reduction as in FIG. 4). Data representative of N=2 donors and error bars are SEM.

FIG. 16 shows representative dot plots showing specific binding of recombinant PSMA protein to mJ591-HiT T cells. (A) TRACKO/Scrambled (SCR) and (B) TRACKO/SUVKO samples incubated with recombinant avi-tagged PSMA protein and stained with streptavidin-A647. The negative control (left) stained only with streptavidin-A647 and no recombinant protein. C) Percentages of PSMA-interacting cells. Data representative of N=2 donors and error bars are SEM.

FIG. 17 shows increased killing of SUV-KO HiT samples compared to scrambled (SCR) controls in kinetic kill assay. TRAC-KO-Scramble and TRAC-KO-SUV-KO or gRV-FMC63-HiT+Booster cells were co-cultured with GFP+ LNCaP-19 target line at E:T of 1:20 in 96-well plates and placed into the InCucyte for GFP detection for 7 days. Untransduced T cells were used as negative controls. Representative graphs from 2 independent experiments and samples are shown. Error bars are SD

FIG. 18 shows increased killing of SUV-KO HiT samples compared to scrambled (SCR) controls in kinetic kill assay. TRAC-KO-Scramble and TRAC-KO-SUV-KO (A) gRV-mJ591-HiT or (B) gRV-mJ591-HiT+Booster cells were co-cultured with GFP+ LNCaP-19 target line at a E:T ratio of 1:20 in a 96-well plate and placed into the InCucyte for GFP detection for 7 days. Untransduced T cells were used as negative controls. Representative graphs from 2 independent experiments and samples are shown/Error bars are SD

FIG. 19 illustrates that SUV-KO cells after a 2^{nd} stimulation in a kinetic killing assay show Increased killing compared to scrambled (SCR) controls. TRAC-KO-Scrambled and TRAC-KO-SUV-KO gRV-FMC63-HiT cells, already undergone a 1^{st} antigen stimulation, were filtered with a 20 uM filter and were FACS analyzed to determine % of HiT+ cells. These gRV-FMC63-HiT+Booster cells were co-cultured with GFP+ LNCap-19 target cells at two different E:T ratios: (A) 1:5 and (B) 1:10 in a 96-well plate and placed into the InCucyte for GFP detection for 7 days. “Target cell only” was used as the negative control for these experiments. Representative graphs from 2 independent experiments and samples are shown. Error bars are SD

FIG. 20 A shows that TRAC-KO-SUV-KO gRV FMC63-HiT+Booster sample have an increased expansion as compared to Scrambled (SCR) controls. TRAC-KO-Scramble and TRAC-KO-SUV-KO gRV-FMC63-HiT+Booster cells were co-cultured with GFP+ LNCap-19 target cells at the indicated E:T ratios in a 96-well plate and placed into the InCucyte for GFP detection for 7 days. After 7 days, co-cultures underwent FACS analysis for cell counting. Data representative of N=2 donors and error bars are SEM. FIG. 20B shows that HiT+booster SUV KO cells have an increased expansion of after 2^{nd} stimulation compared to Scrambled (SCR) controls. TRAC-KO-Scramble and TRAC-KO-SUV-KO gRV-FMC63-HTi+Booster cells were co-cultured with GFP+ LNCap-19 target line at the indicated E:T ratios in a 96-well plate and placed into the InCucyte for GFP detection for 7 days. After the 7 days, co-cultures underwent FACS analysis for cell counting. Data representative of N=2 donors and error bars are SEM.

FIG. 21 shows that SUV-KO gRV HiT cells exhibit improved memory-associated profile, reduced effector-like profile and reduced exhaustion marker phenotype compared to Scrambled (SCR) controls after manufacturing. (A) Phenotype at day 15 after initial T cell activation of gRV TRAC-KO and Scrambled/SUV-KO FMC63-HiT+Booster and (B) gRV mJ591-HiT+booster T cells. Cells were analyzed for surface marker expression under the following gating strategy: (A) and (B) lymphocyte⁺singlet⁺alive⁺CD4^{+/-}CD8^{+/-}Booster⁺CD45RO⁺. CCR7 and CD27 expression were analyzed to check for sternness and broken down accordingly. CM: central memory, Ttm: transitional memory and EM: effector memory. (C) and (D) lymphocyte⁺singlet⁺alive⁺CD4^{+/-}CD8^{+/-}Booster⁺ for CD57 and TIM3 respectively. Data representative of N=2 donors and error bars are SEM.

FIG. 22 shows that SUV-KO gRV HiT cells exhibit improved memory-associated profile, reduced effector-like profile and reduced exhaustion marker phenotype as compared to Scrambled (SCR) controls after 1^{st} antigen stimulation. (A) gRV TRAC-KO and Scrambled/SUV-KO FMC63-HiT+Booster and (B) gRV mJ591-HiT+booster were co-cultured with GFP+ LNCap-19 target line at various E:T ratios. Analysis at day 7 after plating for surface marker expression under the following gating strategy: (A) and (B) lymphocyte⁺singlet⁺alive⁺CD4^{+/-}CD8^{+/-} Booster⁺CD45RO⁺. CCR7 and CD27 expression were analyzed to check for stemness and broken down accordingly. CM: central memory, Ttm: transitional memory and EM: effector memory. (C) and (D) lymphocyte⁺singlet⁺alive⁺CD4^{+/-}CD8^{+/-}Booster⁺ for CD57 and TIM3 respectively. Data representative of N=2 donors and error bars are SEM.

FIG. 23 shows that SUV-KO gRV HiT cells exhibit improved memory-associated profile, reduced effector-like profile and reduced exhaustion marker phenotype compared to Scrambled (SCR) controls after 2nd antigen stimulation. TRAC-KO-Scrambled and TRAC-KO-SUV-KO gRV-HiT+booster cells, already undergone a 1^{st} antigen stimulation, were filtered with a 20-μM filter and were FACS analyzed to determine % of HiT+ cells. These gRV-HiT+Booster cells were co-cultured with GFP+ LNCap-19 target line at a E:T ratio of 1:5 (1.5e³ HIT T-cells and 7.5e³ target cells) in a 96-well plate and placed into the InCucyte for GFP detection for 7 days. (A) and (C) graphs display gRV FMC63-HiT+Booster cells and (B) and (D) graphs display gRV mJ591-HiT+booster cells. Analysis at day 7 after plating for surface marker expression under the following gating strategy: (A) and (B) lymphocyte⁺singlet⁺alive⁺CD4⁺-CD8⁺-Booster⁺CD45RO⁺. CCR7 and CD27 expression were analyzed to check for sternness and broken down accordingly. CM: central memory, Ttm: transitional memory and EM: effector memory. (C) and (D) lymphocyte⁺singlet⁺alive⁺CD4^{+/-}CD8^{+/-}Booster⁺ for CD57 and TIM3 respectively). Data representative of N=2 donors and error bars are SEM.

FIG. 24 shows a schematic of of TRAC-HIT cells generation. An AAV with homology arms is knocked into the TRAC locus following a Crisp/Cas9 mediated double strand break. The DNA repair machinery incorporates the expression cassette of the HIT construct and puts it under the control of the endogenous promoter. The approach is multiplexed with additional Cas9 RNPs to promote SUV39H1 gene editing.

FIG. 25A illustrates HIT reconstitution with increased CD3⁺TCRab⁺ populations in HIT-AAV-treated cells. FIG. 25B shows decreased levels of SUV39H1 protein in SUV39H1 gRNA-treated cells (SUVKO) compared to scramble gRNA treated cells (SCR) as measured by western blotting. FIG. 25C shows decreased levels of H3K9me3 in SUV39H1 gRNA-treated cells (SUVKO) compared to scramble gRNA treated cells (SCR) as measured by flow cytometry.

FIG. 26A shows the expression of 19-HIT by flow cytometry using a biotinylated soluble CD19 molecule. FIG. 26B shows a schematic of the in vitro experimental design. TRAC-19-HIT T cells were co-cultured with different ratios of NALM6 cells with WT levels of CD19 for 10 days. On Day 10, TRAC-19-HIT cell phenotype was analyzed by flow cytometry. FIG. 26C shows flow cytometry analysis of CD27 and CD45RO expression in TRAC-19-HIT cells. The percentage of CD27+ cells is shown.

FIG. 27A shows a schematic of in vivo experimental design. NALM6 cells with WT levels of CD19 were injected in NSG mice on Day 0. TRAC-19-HIT cells either sufficient (SCR) or (SUVKO) were then injected on Day 3. FIG. 27B shows tumor growth monitored by bioluminescence imaging (average radiance, photons/sec/cm²). C) Percent survival of TRAC-19-HIT treated mice.

FIG. 28A shows a schematic of in vivo experimental design. NALM6 cells with Low levels of CD19 were injected in NSG mice on Day 0. TRAC-19-HIT cells either sufficient (SCR) or (SUVKO) were then injected on Day 3. B) Tumor growth monitored by bioluminescence imaging (average radiance, photons/sec/cm²).

FIGS. 29-30 illustrate example of SFG (gamma retro viral) HIT constructs directed against the mesothelin tumor antigen and comprising a VH fragment fused to a TRBC2 sequence and a VL fragment fused to a TRAC sequence. Booster sequence (CCR) can be further made recombinantly expressed. The booster sequence can be provided in a separate construct (expression cassette) or in the same construct as illustrated in FIG. 29. In some embodiment the booster (CCR) sequence is a CD80_4-1BB chimeric receptor as illustrated inn SEQ ID NO: 33.

### DETAILED DESCRIPTION

### Definitions

As used herein, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of +10%, 5%, 1%, ±0.5%, or even ±0.1% of the specified amount.

The term “antigen recognizing receptor” as used herein refers to a receptor that is capable of activating an immune or immunoresponsive cell (e.g., a T-cell) in response to its binding to an antigen. Non-limiting examples of antigen recognizing receptors include native or endogenous T cell receptors (“TCRs”), and chimeric antigen receptors (“CARs”).

The term “antibody” herein is used in the broadest sense and includes polyclonal and monoclonal antibodies, including intact antibodies, chimeric, human or humanized antibodies, and functional (antigen-binding) antibody fragments, including fragment antigen binding (Fab) fragments, F(ab′)2 fragments, Fab′ fragments, Fv fragments, recombinant IgG (rIgG) fragments, variable heavy chain (VH) regions capable of specifically binding the antigen, single chain antibody fragments, including single chain variable fragments (scFv), and single domain antibodies (e.g., sdAb, sdFv, nanobody) fragments. The term encompasses recombinant and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgG1, IgG2, IgG3, IgG4, IgM, IgE, IgA, and IgD. In some embodiments the antibody comprises a heavy chain variable region and a light chain variable region.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; variable heavy chain (VH) regions, VHH antibodies, single-chain antibody molecules such as scFvs and single-domain antibodies (including VH and VL single antibodies); and multispecific antibodies formed from antibody fragments. In particular embodiments, the antibodies are single-chain antibody fragments comprising a variable heavy chain region and/or a variable light chain region, such as scFvs.

“Single-domain antibodies” are antibody fragments comprising all or a portion of the heavy chain variable domain or all or a portion of the light chain variable domain of an antibody. In certain embodiments, a single-domain antibody is a human single-domain antibody.

“Inactivation” or “disruption” of a gene refers to a change in the sequence of genomic DNA that causes the gene's expression to be reduced or eliminated, or that cause a non-functional gene product to be expressed. Exemplary methods include gene silencing, knockdown, knockout, and/or gene disruption techniques, such as gene editing through, e.g., induction of breaks and/or homologous recombination. Exemplary of such gene disruptions are insertions, frameshift and missense mutations, deletions, knock-in, and knock-out of the gene or part of the gene, including deletions of the entire gene. Such disruptions can occur in the coding region, e.g., in one or more exons, resulting in the inability to produce a full-length product, functional product, or any product, such as by insertion of a stop codon. Such disruptions may also occur by disruptions in the promoter or enhancer or other region affecting activation of transcription, so as to prevent transcription of the gene. Gene disruptions include gene targeting, including targeted gene inactivation by homologous recombination.

“Inhibition” or “repression” of a gene refers to a decrease of activity and/or gene expression of at least 30%, 40%, 50%, 60%, 70%, 80%, 90%, 95% or 99% or more as compared to the activity or expression levels of wildtype which is not inhibited or repressed. The inhibition of gene expression leads to the absence in the cell of substantial detectable activity or functional gene product.

“Non-functional” refers to a protein with reduced activity or a lack of detectable activity.

“Dominant negative” refers to a mutation which produces a defective gene, and said defective gene interferes or adversely affects the function of the wildtype product within the same cell. The ability of the defective gene to interact with the same elements as the wildtype product remains, but some functional aspects are blocked.

“Express” or “expression” means that a gene sequence is transcribed, and optionally, translated. If the gene expresses a noncoding RNA, expression will typically result in an RNA after transcription and, optionally, splicing. If the gene is a coding sequence, expression will typically result in production of a polypeptide after transcription and translation.

“Expression control sequence” refers to a nucleotide sequence that influences the transcription, RNA processing, RNA stability, or translation of the associated nucleotide sequence. Examples include, but are not limited to, promoters, enhancers, introns, translation leader sequences, polyadenylation signal sequences, transcription initiators and transcriptional and/or translational termination region (i.e., termination region). “Fragment” refers to a portion of a referenced sequence (polynucleotide or polypeptide) that has a length at least 60%, 65%, 70%, 75%, 80%, 85%, 90%, 95%, 96%, 97%, 98%, or 99% of the full-length sequence.

By“exogenous” is meant a nucleic acid molecule (e.g., a cDNA, DNA or RNA molecule) or polypeptide that is not endogenously present in a cell, or not present at a level sufficient to achieve the functional effects obtained when over-expressed. The term“exogenous” would therefore encompass any recombinant nucleic acid molecule or polypeptide expressed in a cell, such as foreign, heterologous, and over-expressed nucleic acid molecules and polypeptides. By“exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non native promoter or secretory sequence.

“Heterologous” refers to a polynucleotide or polypeptide that comprises sequences that are not found in the same relationship to each other in nature. For example, the heterologous sequence either originates from another species, or is from the same species or organism but is modified from either its original form or the form primarily expressed in the cell. Thus, a heterologous polynucleotide includes a nucleotide sequence derived from and inserted into the same natural, original cell type, but which is present in a non-natural state, e.g., a different copy number, and/or under the control of different regulatory sequences than that found in nature and/or located in a different position (adjacent to a different nucleotide sequence) than where it was originally located.

“Nucleic acid,” “nucleotide sequence,” and “polynucleotide” are used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., modified backbone, sugars or bases). Such oligonucleotides can be used, for example, to prepare nucleic acids that have altered base-pairing abilities or increased resistance to nucleases. The present disclosure further provides a nucleic acid that is the complement (which can be either a full complement or a partial complement) of a nucleic acid, nucleotide sequence, or polynucleotide described herein. Modified bases (modified nucleobases), such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

“Operably linked” means that an element, such as an expression control sequence, is configured so as to perform its usual function upon a nucleotide sequence of interest. For example, a promoter operably linked to a nucleotide sequence of interest is capable of effecting expression of the nucleotide sequence of interest. The expression control sequences need not be contiguous with the nucleotide sequence of interest, so long as they function to direct the expression thereof.

“Percent identity” between two sequences, means the percentage of identical bases or amino acids between the two sequences to be compared, obtained with the best alignment of said sequences, this percentage being purely statistical and the differences between these two sequences being randomly spread over the two sequences. A base is considered complementary if it hybridizes under normal conditions; for example, a modified nucleobase can be aligned in a manner like the base whose hybridization pattern it mimics. As used herein, “best alignment” or “optimal alignment”, means the alignment for which the determined percentage of identity (see below) is the highest. Sequence comparison between two nucleic acid sequences (also referenced herein as nucleotide sequence or nucleobase sequence) is usually realized by comparing these sequences that have been previously aligned according to the best alignment; this comparison is realized on segments of comparison in order to identify and compared the local regions of similarity. The best sequences alignment to perform comparison can be realized, besides manually, by using the global homology algorithm developed by SMITH and WATERMAN (Ad. App. Math., vol. 2, p: 482, 1981), by using the local homology algorithm developed by NEDDLEMAN and WUNSCH (J. Mol. Biol, vol. 48, p: 443, 1970), by using the method of similarities developed by PEARSON and LIPMAN (Proc. Natl. Acd. Sci. USA, vol. 85, p: 2444, 1988), by using computer softwares using such algorithms (GAP, BESTFIT, BLAST P, BLAST N, FASTA, TFASTA in the Wisconsin Genetics software Package, Genetics Computer Group, 575 Science Dr., Madison, WI USA), by using the MUSCLE multiple alignment algorithms (Edgar, Robert C, Nucleic Acids Research, vol. 32, p: 1792, 2004). To get the best local alignment, one can preferably use BLAST software. The identity percentage between two sequences is determined by comparing these two sequences optimally aligned, the sequences being able to comprise additions or deletions in respect to the reference sequence in order to get the optimal alignment between these two sequences. The percentage of identity is calculated by determining the number of identical positions between these two sequences, and dividing this number by the total number of compared positions, and by multiplying the result obtained by 100 to get the percentage of identity between these two sequences.

By“immunoresponsive cell” is meant a cell that functions in an immune response or a progenitor, or progeny thereof.

Treatment”, or “treating” as used herein, is defined as the application or administration of cells as per the disclosure or of a composition comprising the cells to a patient in need thereof with the purpose to cure, heal, alleviate, relieve, alter, remedy, ameliorate, improve or affect the disease such as cancer, or any symptom of the disease (e.g., cancer). In particular, the terms “treat” or treatment” refers to reducing or alleviating at least one adverse clinical symptom associated with the disease such as the cancer cancer, e.g., pain, swelling, low blood count etc. With reference to cancer treatment, the term “treat” or treatment” also refers to slowing or reversing the progression neoplastic uncontrolled cell multiplication, i.e. shrinking existing tumors and/or halting tumor growth. The term “treat” or treatment” also refers to inducing apoptosis in cancer or tumor cells in the subject.

“Variant” refers to a sequence (polynucleotide or polypeptide) that has mutations (deletion, substitution or insertion) that is at least 60%, 65%, 70%, 75%, 80%, 85%, 90%, 95%, 96%, 97%, 98%, or 99% identical to a referenced sequence over its full length or over a region of at least about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, or 1100 nucleotides or amino acids. With respect to a polynucleotide sequence, variant also encompasses a polynucleotide that hybridizes under stringent conditions to the referenced sequence, or complement thereof.

By “modulate” is meant positively or negatively alter. Exemplary modulations include a about 1%, about 2%, about 5%, about 10%, about 25%, about 50%, about 75%, or about 100% change.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

By “effective amount” is meant an amount sufficient to have a therapeutic effect. In certain embodiments, an“effective amount” is an amount sufficient to arrest, ameliorate, or inhibit the continued proliferation, growth, or metastasis (e.g., invasion, or migration) of a neoplasia.

By “isolated cell” is meant a cell that is separated from the molecular and/or cellular components that naturally accompany the cell.

The terms “isolated,” “purified,” or“biologically pure” refer to material that is free to varying degrees from components which normally accompany it as found in its native state. “Isolate” denotes a degree of separation from original source or surroundings. “Purify” denotes a degree of separation that is higher than isolation. A “purified” or“biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term“purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

The term “antigen-binding domain” as used herein refers to a domain capable of specifically binding a particular antigenic determinant or set of antigenic determinants present on a cell.

“Linker”, as used herein, shall mean a functional group (e.g., chemical or polypeptide) that covalently attaches two or more polypeptides or nucleic acids so that they are connected to one another. As used herein, a“peptide linker” refers to one or more amino acids used to couple two proteins together (e.g., to couple VH and VL domains). In certain embodiments, the linker comprises a sequence set forth in GGGGSGGGGSGGGGS [SEQ ID NO: 19]

As used herein, a “vector” is any nucleic acid molecule for the transfer into or expression of a nucleic acid in a cell. The term “vector” includes both viral and nonviral (e.g., plasmid) nucleic acid molecules for introducing a nucleic acid into a cell in vitro, ex vivo, and/or in vivo. Vectors may include expression control sequences, restriction sites, and/or selectable markers. A “recombinant” vector refers to a vector that comprises one or more heterologous nucleotide sequences.

By “signal sequence” or “leader sequence” is meant a peptide sequence (e.g., 5, 10, 15, 20, 25 or 30 amino acids) present at the N-terminus of newly synthesized proteins that directs their entry to the secretory pathway. Exemplary leader sequences include, but is not limited to, the IL-2 signal sequence: MYRMQLLSCIALSLALVTNS [SEQ ID NO: 20](human), MY SMQLASC VTLTLVLLVN S [SEQ ID NO: 21](mouse); the kappa leader sequence: METP AQLLFLLLLWLPDTT G [SEQ ID NO: 22](human), METDTLLLW VLLLW VPGS T G [SEQ ID NO: 23](mouse); the CD8 leader sequence: M ALP VT ALLLPL ALLLH A ARP [SEQ ID NO: 24](human); the truncated human CD8 signal peptide: M ALP VT ALLLPL ALLLH A [SEQ ID NO: 25](human); the albumin signal sequence: MKWVTFISLLFSSAYS [SEQ ID NO: 26](human); and the prolactin signal sequence: MD SKGS SQKGSRLLLLLVV SNLLLCQGVV S [SEQ ID NO: 27](human). By “soluble” is meant a polypeptide that is freely diffusible in an aqueous environment (e.g., not membrane bound).

By “specifically binds” is meant a polypeptide or fragment thereof that recognizes and binds to a biological molecule of interest (e.g., a polypeptide), but which does not substantially recognize and bind other molecules in a sample, for example, a biological sample, which naturally includes a presently disclosed polypeptide.

The term “tumor antigen” as used herein refers to an antigen (e.g., a polypeptide) that is uniquely or differentially expressed on a tumor cell compared to a normal or non-IS neoplastic cell. In certain embodiments, a tumor antigen includes any polypeptide expressed by a tumor that is capable of activating or inducing an immune response via an antigen recognizing receptor (e.g., CD19, MUC-16) or capable of suppressing an immune response via receptor-ligand binding (e.g., CD47, PD-L1/L2, B7.1/2).

The expression booster has been used herein for costimulatory ligand and costimulatory receptor (CCR). In certain embodiments, the co-stimulatory ligand can be selected from the group consisting of CD80, CD86, 41 BBL, CD275, CD40L, OX40L and any combination thereof. In certain embodiments, the cell further comprises or consists of one exogenous co-stimulatory ligand. In certain embodiments, the one exogenous co-stimulatory ligand is CD80 or 4-1BBL. In certain embodiments, the cell further comprises or consists of two exogenous co-stimulatory ligands. In certain embodiments, In certain embodiments, the two exogenous co-stimulatory ligands are CD80 and 4-1BBL. In certain embodiments, the immunoresponsive cell comprises at least one chimeric costimulatory receptor (CCR). In certain embodiments, the CCR comprising a co-stimulatory molecule selected from the group consisting of a CD80 polypeptide, a CD28 polypeptide, a 4-1BB polypeptide, an OX40 polypeptide, an ICOS polypeptide, a DAP-10 polypeptide and any combination thereof. Example co-stimulatory ligands, molecules and receptors (or fusion polypeptides) are described in Int'l Pat. Pub. No. WO-2021/016174, incorporated by reference herein in its entirety. Illustrative booster sequences include SEQ ID NO: 32-33 and 53-54.

### Cells

### Immune Cells (Immuno-Responsive Cells)

The immune cells according to the disclosure are typically mammalian cells, e.g., human cells.

More particularly, the cells of the disclosure are derived from the blood, bone marrow, lymph, or lymphoid organs (notably the thymus) and are cells of the immune system (i.e., immune cells), such as cells of the innate or adaptive immunity, e.g., myeloid or lymphoid cells, including lymphocytes, typically T cells and/or NK cells.

Preferably according to the disclosure, cells are notably lymphocytes including T cells, B cells, NK cells and progenitors thereof.

Cells according to the disclosure may also be immune cell progenitors, such as lymphoid progenitors and more preferably T cell progenitors. Examples of T-cell progenitors include pluripotent stem cells (PSCs), induced pluripotent stem cells (iPSCs), hematopoietic stem cells (HSCs), human embryonic stem cells (ESCs), adipocyte-derived stem cells (ADSCs), multipotent progenitor (MPP); lymphoid-primed multipotent progenitor (LMPP); common lymphoid progenitor (CLP); lymphoid progenitor (LP); thymus settling progenitor (TSP); or early thymic progenitor (ETP). Hematopoietic stem and progenitor cells can be obtained, for example, from cord blood, or from peripheral blood, e.g. peripheral blood-derived CD34+ cells after mobilization treatment with granulocyte-colony stimulating factor (G-CSF).

T cell progenitors typically express a set of consensus markers including CD44, CD117, CD135, and/or Sca-1 but see also Petrie H T, Kincade P W. Many roads, one destination for T cell progenitors. The Journal of Experimental Medicine. 2005; 202(1):11-13.

The cells typically are primary cells, such as those isolated directly from a subject and/or isolated from a subject and frozen.

With reference to the subject to be treated, the cells of the disclosure may be allogeneic and/or autologous.

In autologous immune cell therapy, immune cells are collected from the patient, modified as described herein, and returned to the patient. In allogeneic immune cell therapy, immune cells are collected from healthy donors, rather than the patient, modified as described herein, and administered to patients. Typically these are HLA matched to reduce the likelihood of rejection by the host. The immune cells may also comprise modifications such as disruption or removal of HLA class I molecules. For example, Torikai et al., Blood. 2013; 122:1341-1349 used ZFNs to knock out the HLA-A locus, while Ren et al., Clin. Cancer Res. 2017; 23:2255-2266 knocked out Beta-2 microglobulin (B2M), which is required for HLA class I expression.

In addition, universal ‘off the shelf’ product immune cells must comprise modifications designed to reduce graft vs. host disease, such as inactivation (e.g. disruption or deletion) of the TCRαβ receptor; the resulting cell exhibits significantly reduced or nearly eliminated expression of the endogenous TCR. See Graham et al., Cells. 2018 October; 7(10): 155 for a review. Because a single gene encodes the alpha chain (TRAC) rather than the two genes encoding the beta chain, the TRAC locus is a typical target for removing or disrupting TCRαβ receptor expression, although the TCRβ loci may alternatively be disrupted. Alternatively, inhibitors of TCRαβ signaling may be expressed, e.g. truncated forms of CD3zeta can act as a TCR inhibitory molecule. Ren et al. simultaneously knocked out TCRαβ, B2M and the immune-checkpoint PD1.

In some embodiments, the cells include one or more subsets of T cells or other cell types, such as whole T cell populations, CD4+ cells and/or CD8+ cells, and subpopulations thereof, such as those defined by function, activation state, maturity, potential for differentiation, expansion, recirculation, localization, and/or persistence capacities, antigen-specificity, type of antigen-specific receptor, presence in a particular organ or compartment, marker or cytokine secretion profile, and/or degree of differentiation.

Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. Preferably, the cells according to the disclosure are TEFF cells with stem/memory properties and higher reconstitution capacity due to the inhibition of Suv39h1, as well as TN cells, TSCM, TCM, TEM cells and combinations thereof.

In some embodiments, one or more of the T cell populations is enriched for, or depleted of, cells that are positive for or express high levels of one or more particular markers, such as surface markers, or that are negative for or express relatively low levels of one or more markers. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD117, CD135, CD45RO, CCR7, CD28, CD27, CD44, CD127, and/or CD62L and/or depleted of (e.g., negatively selected for) cells that are positive for or express high surface levels of CD45RA. In some embodiments, cells are enriched for or depleted of cells positive or expressing high surface levels of CD122, CD95, CD25, CD27, and/or IL7-Ra (CD127). In some examples, CD8+ T cells are enriched for cells positive for CD45RO (or negative for CD45RA) and for CD62L. The subset of cells that are CCR7+, CD45RO+, CD27+, CD62L+ cells constitute a central memory cell subset.

For example, according to the disclosure, the cells can include a CD4+ T cell population and/or a CD8+ T cell sub-population, e.g., a sub-population enriched for central memory (TCM) cells. Alternatively, the cells can be other types of lymphocytes, including natural killer (NK) cells, mucosal associated invariant T (MAIT) cells, Innate Lymphoid Cells (ILCs) and B cells.

The cells and compositions containing the cells for engineering according to the disclosure are isolated from a sample, notably a biological sample, e.g., obtained from or derived from a subject. Typically, the subject is in need for a cell therapy (adoptive cell therapy) and/or is the one who will receive the cell therapy. The subject is preferably a mammal, notably a human. In one embodiment of the disclosure, the subject has a cancer.

The samples include tissue, fluid, and other samples taken directly from the subject, as well as samples resulting from one or more processing steps, such as separation, centrifugation, genetic engineering (for example transduction with viral vector), washing, and/or incubation. The biological sample can be a sample obtained directly from a biological source or a sample that is processed. Biological samples include, but are not limited to, tissue samples, from tissues or organ, or fluid samples, such as blood, plasma, serum, cerebrospinal fluid, or synovial fluid, including processed samples derived therefrom. Preferably, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, and/or cells derived therefrom. Samples include, in the context of cell therapy (typically adoptive cell therapy) samples from autologous and allogeneic sources.

In some embodiments, the cells are derived from cell lines, e.g., T cell lines. The cells can also be obtained from a xenogeneic source, such as a mouse, a rat, a non-human primate, or a pig. Preferably, the cells are human cells.

The presently disclosed subject matter provides immunoresponsive cells comprising a presently disclosed HI-TCR. In certain embodiments, the HI-TCR is capable of activating the immunoresponsive cell. Upon binding to the antigen, the immunoresponsive cells exhibit cytolytic effects towards cells bearing the antigen. In certain embodiments, the immunoresponsive cells comprising the HI-TCR exhibits comparable or better therapeutic potency compared to cells comprising a chimeric antigen receptor (CAR) targeting the same antigen. In certain embodiments, the immunoresponsive cells comprising the HI-TCR exhibit comparable or better cytolytic effects compared to cells comprising a chimeric antigen receptor (CAR) targeting the same antigen. In certain embodiments, the immunoresponsive cells comprising the HI-TCR secrete anti-tumor cytokines. The cytokines secreted by the immunoresponsive cells include, but are not limited to, TNFa, IFNy and IL2.

Typically, according to the present subject matter, the immune cell (immunoresponsive cell) is defective for Suv39h1.

Human Suv39h1 methyltransferase is referenced as 043463 in UNIPROT and is encoded by the gene Suv39h1 located on chromosome x (gene ID: 6839 in NCBI). One exemplary human gene sequence is SEQ ID NO: 15, and one exemplary human protein sequence is SEQ ID NO: 16, but it is understood that polymorphisms or variants with different sequences exist in various subjects' genomes. The term Suv39h1 according to the disclosure thus encompasses all mammalian variants of SUV39H1, and genes that encode a protein at least 75%, 80%, or typically 85%, 90%, or 95% identical to SEQ ID NO: 16 that has SUV39H1 activity (i.e., the methylation of Lys-9 of histone H3 by H3K9-histone methyltransferase).

As used herein the expression “defective for Suv39h1” according to the present invention refers to the inhibition, or blockade of Suv39h1 activity (i.e., the methylation of Lys-9 of histone H3 by H3K9-histone methyltransferase) in the cell according to the invention.

“Inhibition of Suv39h1 activity” as per the invention refers to a decrease of Suv39h1 activity of at least 30%, 40%, 50%, 60%, 70%, 80%, 90%, 95% or 99% or more as compared to the activity or level of the Suv39h1 protein which is not inhibited. Preferentially, the inhibition of Suv39h1 activity leads to the absence in the cell of substantial detectable activity of Suv39h1.

Inhibition of Suv39h1 activity can also be achieved through repression of Suv39h1 gene expression or though Suv39h1 gene disruption. According to the invention, said repression reduces expression of Suv39h1 in the cell, notably the immune cell of the invention by at least 50, 60, 70, 80, 90, or 95% as to the same cell produced by the method in the absence of the repression. Gene disruption may also lead to a reduced expression of the Suv39h1 protein or to the expression of a non-functional Suv39h1 protein.

By “non-functional” Suv39h1 protein it is herein intended a protein with a reduced activity or a lack of detectable activity as described above. Thus inhibitors of Suv39h1 activity in a cell according to the invention can be selected among any compound or agent natural or not having the ability of inhibiting the methylation of Lys-9 of histone H3 by H3K9-histone methyltransferase, or inhibiting the H3K9-histone methyltransferase SUV39H1 gene expression. Inhibition of Suv39h1 in the immune cell according to the present invention can be permanent and irreversible or transient or reversible. Preferably however, Suv39h1 inhibition is permanent and irreversible. Inhibition of Suv39h1 in the cell may be achieved prior or after injection of the cell in the targeted patient as described below.

### Antigen-Specific Receptors

In some embodiments, the immune cells express antigen-specific receptors on the surface. The cells thus may comprise one or more nucleic acids that encode one or more antigen-specific receptors, optionally operably linked to a heterologous regulatory control sequence. Typically such antigen-specific receptors bind the target antigen with a Kd binding affinity of 10⁻⁶M or less, 10⁻⁷ M or less, 10⁻⁸ M or less, 10⁻⁹ M or less, 10⁻¹⁰ M or less, or 10⁻¹¹ M or less (lower numbers indicating greater binding affinity). In some embodiments, the antigen-binding domain binds a target antigen with a KD affinity of about 1×10⁻⁷ or less, about 5×10⁻⁸ or less, about 1×10⁻⁸ or less, about 5×10⁻⁹ or less, about 1×10⁻⁹ or less, about 5×10⁻¹⁰ or less, about 1×10⁻¹⁰ or less, about 5×10⁻¹¹ or less, about 1×10⁻¹¹ or less, about 5×10⁻¹² or less, or about 1×10⁻¹² or less.

Typically, the nucleic acids are exogenous (e.g. heterologous), (i.e., for example which are not ordinarily found in the cell being engineered and/or in the organism from which such cell is derived). In some embodiments, the nucleic acids are not naturally occurring, including chimeric combinations of nucleic acids encoding various domains from multiple different cell types. The nucleic acids and their regulatory control sequences are typically heterologous. For example, the nucleic acid encoding the antigen-specific receptor may be heterologous to the immune cell and operatively linked to an endogenous promoter of the T-cell receptor such that its expression is under control of the endogenous promoter. In some embodiments, the nucleic acid encoding a modified TCR or CAR is operatively linked to an endogenous TRAC promoter.

Among the antigen-specific receptors as per the disclosure are recombinant modified T cell receptors (TCRs) and components thereof, as well as functional non-TCR antigen-specific receptors, such as chimeric antigen receptors (CAR).

The immune cells, particularly if allogeneic, may be designed to reduce graft vs. host disease, such that the cells comprise inactivated (e.g. disrupted or deleted) TCRαβ receptor. Because a single gene encodes the alpha chain (TRAC) rather than the two genes encoding the beta chain, the TRAC locus is a typical target for reducing TCRαβ receptor expression. Thus, the nucleic acid encoding the antigen-specific receptor (e.g. CAR or TCR) may be integrated into the TRAC locus at a location, preferably in the 5′ region of the first exon (SEQ ID NO: 17), that significantly reduces expression of a functional TCR alpha chain. See, e.g., Jantz et al., WO 2017/062451; Sadelain et al., WO 2017/180989; Torikai et al., Blood, 119(2): 5697-705 (2012); Eyquem et al., Nature. 2017 Mar. 2; 543(7643):113-117. Expression of the endogenous TCR alpha may be reduced by at least 50%, 60%, 70%, 75%, 80%, 85%, 90%, 95%, 98%, or 99%. In such embodiments, expression of the nucleic acid encoding the antigen-specific receptor is optionally under control of the endogenous TCR-alpha promoter.

### Chimeric Antigen Receptors (CARs)

In some embodiments, the engineered antigen-specific receptors comprise chimeric antigen receptors (CARs), including activating or stimulatory CARs, costimulatory CARs (see WO2014/055668), and/or inhibitory CARs (iCARs, see Fedorov et al., Sci. Transl. Medicine, 5(215) (December, 2013)).

Chimeric antigen receptors (CARs), (also known as Chimeric immunoreceptors, Chimeric T cell receptors, Artificial T cell receptors) are engineered antigen-specific receptors, which graft an arbitrary specificity onto an immune effector cell (T cell). Typically, these receptors are used to graft the specificity of a monoclonal antibody onto a T cell, with transfer of their coding sequence facilitated by retroviral vectors.

CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

The CAR may include
(a) an extracellular antigen-binding domain,
(b) a transmembrane domain,
(c) optionally a co-stimulatory domain, and
(d) an intracellular signaling domain.

In some embodiments, the CAR is constructed with a specificity for a particular antigen (or marker or ligand), such as an antigen expressed in a particular cell type to be targeted by adoptive cell therapy, such as a cancer marker. The CAR typically includes in its extracellular portion one or more antigen binding molecules, such as one or more antigen-binding fragment, domain, or portion of an antibody, typically one or more antibody variable domains. For example, the extracellular antigen-binding domain may comprise a light chain variable domain and a heavy chain variable domain, typically as an scFv.

The moieties used to bind to antigen include three general categories, either single-chain antibody fragments (scFvs) derived from antibodies, Fab's selected from libraries, or natural ligands that engage their cognate receptor (for the first-generation CARs). Successful examples in each of these categories are notably reported in Sadelain M, Brentjens R, Riviere I. The basic principles of chimeric antigen receptor (CAR) design. Cancer discovery. 2013; 3(4):388-398 (see notably table 1) and are included in the present application.

Antibodies include chimeric, humanized or human antibodies, and can be further affinity matured and selected as described above. Chimeric or humanized scFv's derived from rodent immunoglobulins (e.g. mice, rat) are commonly used, as they are easily derived from well-characterized monoclonal antibodies. Humanized antibodies contain rodent-sequence derived CDR regions; typically the rodent CDRs are engrafted into a human framework, and some of the human framework residues may be back-mutated to the original rodent framework residue to preserve affinity, and/or one or a few of the CDR residues may be mutated to increase affinity. Fully human antibodies have no murine sequence, and are typically produced via phage display technologies of human antibody libraries, or immunization of transgenic mice whose native immunoglobin loci have been replaced with segments of human immunoglobulin loci. Variants of the antibodies can be produced that have one or more amino acid substitutions, insertions, or deletions in the native amino acid sequence, wherein the antibody retains or substantially retains its specific binding function. Conservative substitutions of amino acids are well known and described above. Further variants may also be produced that have improved affinity for the antigen.

Typically, the CAR includes an antigen-binding portion or portions of an antibody molecule, such as a single-chain antibody fragment (scFv) derived from the variable heavy (VH) and variable light (VL) chains of a monoclonal antibody (mAb). In some embodiments, the CAR comprises an antibody heavy chain variable domain that specifically binds the antigen, such as a cancer marker or cell surface antigen of a cell or disease to be targeted, such as a tumor cell or a cancer cell, such as any of the target antigens described herein or known in the art.

In some embodiments, the CAR contains an antibody or an antigen-binding fragment (e.g. scFv) that specifically recognizes an antigen, such as an intact antigen, expressed on the surface of a cell.

In some embodiments, the CAR contains a TCR-like antibody, such as an antibody or an antigen-binding fragment (e.g. scFv) that specifically recognizes an intracellular antigen, such as a tumor-associated antigen, presented on the cell surface as a MHC-peptide complex. In some embodiments, an antibody or antigen-binding portion thereof that recognizes an MHC-peptide complex can be expressed on cells as part of a recombinant receptor, such as an antigen-specific receptor. Among the antigen-specific receptors are functional non-TCR antigen-specific receptors, such as chimeric antigen receptors (CARs). Generally, a CAR containing an antibody or antigen-binding fragment that exhibits TCR-like specificity directed against peptide-MHC complexes also may be referred to as a TCR-like CAR.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that is naturally associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain can be derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD 134, CD137, CD154, ICOS or a GITR or NKG2D, OX40, 2B4, DAP10, DAP12, or CD40. For T cells, CD8, CD28, CD3 epsilon may be preferred. For NK cells, NKG2D, DAP10, DAP12 may be preferred. The transmembrane domain can also be synthetic. In some embodiments, the transmembrane domain is derived from CD28, CD8 or CD3zeta.

In some embodiments, a short oligo- or polypeptide linker, for example, a linker of between 2 and 10 amino acids in length, is present and forms a linkage between the transmembrane domain and the cytoplasmic signaling domain of the CAR.

The CAR generally includes at least one intracellular signaling component or components, e.g. from the intracellular signaling domain of TCR gamma, delta, epsilon or zeta chain of the T-cell receptor, CD28, CD3 epsilon, CD3zeta, CD45, CD4, CD5, CD8, CD9, CD16, CD22, CD33, CD37, CD64, CD80, CD86, CD 134, CD137, CD154, ICOS or a GITR or NKG2D, OX40, 2B4, DAP10, DAP12, or CD40. For T cells, CD8, CD28, CD3 epsilon may be preferred. For NK cells, NKG2D, DAP10, DAP12 may be preferred. First generation CARs typically had the intracellular domain from the CD3zeta-chain, which is the primary transmitter of signals from endogenous TCRs. Second generation CARs typically further comprise intracellular signaling domains from various costimulatory protein receptors (e.g., CD28, 41 BB (CD28), ICOS) to the cytoplasmic tail of the CAR to provide additional signals to the T cell. Co-stimulatory domains include domains derived from human CD28, 4-1BB (CD137), ICOS-1, CD27, OX 40 (CD137), DAP10, and GITR (AITR). Combinations of two co-stimulatory domains are contemplated, e.g. CD28 and 4-1BB, or CD28 and OX40. Third generation CARs combine multiple signaling domains, such as CD3zeta-CD28-4-1BB or CD3zeta-CD28-OX40, to augment potency.

The intracellular signaling domain can be from an intracellular component of the TCR complex, such as a TCR CD3+ chain that mediates T-cell activation and cytotoxicity, e.g., the CD3zeta chain. Alternative intracellular signaling domains include FcεRIγ. The intracellular signaling domain may comprise a modified CD3zeta polypeptide lacking one or two of its three immunoreceptor tyrosine-based activation motifs (ITAMs), wherein the ITAMs are ITAM1, ITAM2 and ITAM3 (numbered from the N-terminus to the C-terminus). The mature region of CD3zeta is residues 22-164 of SEQ ID NO: 7. ITAM1 is located around amino acid residues 61-89, ITAM2 around amino acid residues 100-128, and ITAM3 around residues 131-159. Thus, the modified CD3zeta polypeptide may have any one of ITAM1, ITAM2, or ITAM3 inactivated. Alternatively, the modified CD3zeta polypeptide may have any two ITAMs inactivated, e.g. ITAM2 and ITAM3, or ITAM1 and ITAM2. Preferably, ITAM3 is inactivated, e.g. deleted. More preferably, ITAM2 and ITAM3 are inactivated, e.g. deleted, leaving ITAM1. For example, one modified CD3zeta polypeptide retains only ITAM1 and the remaining CD3zeta domain is deleted (residues 90-164). As another example, ITAM1 is substituted with the amino acid sequence of ITAM3, and the remaining CD3zeta domain is deleted (residues 90-164). See, for example, Bridgeman et al., Clin. Exp. Immunol. 175(2): 258-67 (2014); Zhao et al., J. Immunol. 183(9): 5563-74 (2009); Maus et al., WO 2018/132506; Sadelain et al., WO/2019/133969, Feucht et al., Nat Med. 25(1):82-88 (2019).

Thus, in some aspects, the antigen binding molecule is linked to one or more cell signaling modules. In some embodiments, cell signaling modules include CD3 transmembrane domain, CD3 intracellular signaling domains, and/or other CD transmembrane domains. The CAR can also further include a portion of one or more additional molecules such as Fc receptor γ, CD8, CD4, CD25, or CD16.

In some embodiments, upon ligation of the CAR, the cytoplasmic domain or intracellular signaling domain of the CAR activates at least one of the normal effector functions or responses of the corresponding non-engineered immune cell (typically a T cell). For example, the CAR can induce a function of a T cell such as cytolytic activity or T-helper activity, secretion of cytokines or other factors.

In some embodiments, the intracellular signaling domain(s) include the cytoplasmic sequences of the T cell receptor (TCR), and in some aspects also those of co-receptors that in the natural context act in concert with such receptor to initiate signal transduction following antigen-specific receptor engagement, and/or a variant of such molecules, and/or any synthetic sequence that has the same functional capability.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

In some aspects, the CAR includes a primary cytoplasmic signaling sequence that regulates primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR zeta, FcR gamma, FcR beta, CD3 gamma, CD3 delta, CD3 epsilon, CDS, CD22, CD79a, CD79b, and CD66d. In some embodiments, cytoplasmic signaling molecule(s) in the CAR contain(s) a cytoplasmic signaling domain, portion thereof, or sequence derived from CD3zeta.

The CAR can also include a signaling domain and/or transmembrane portion of a costimulatory receptor, such as CD28, 4-1BB, OX40, DAP10, and ICOS. In some aspects, the same CAR includes both the activating and costimulatory components; alternatively, the activating domain is provided by one CAR whereas the costimulatory component is provided by another CAR recognizing another antigen.

The CAR or other antigen-specific receptor can also be an inhibitory CAR (e.g. iCAR) and includes intracellular components that dampen or suppress a response, such as an immune response. Examples of such intracellular signaling components are those found on immune checkpoint molecules, including PD-1, CTLA4, LAG3, BTLA, OX2R, TIM-3, TIGIT, LAIR-1, PGE2 receptors, EP2/4 Adenosine receptors including A2AR. In some aspects, the engineered cell includes an inhibitory CAR including a signaling domain of or derived from such an inhibitory molecule, such that it serves to dampen the response of the cell. Such CARs are used, for example, to reduce the likelihood of off-target effects when the antigen recognized by the activating receptor, e.g, CAR, is also expressed, or may also be expressed, on the surface of normal cells.

TCRs and Modified Version Thereof (e.g. Hi-TCR)

In some embodiments, the antigen-specific receptors include recombinant T cell receptors (TCRs) and/or TCRs cloned from naturally occurring T cells. Nucleic acid encoding the TCR can be obtained from a variety of sources, such as by polymerase chain reaction (PCR) amplification of naturally occurring TCR DNA sequences, followed by expression of antibody variable regions, followed by selecting for specific binding to antigen. In some embodiments, the TCR is obtained from T-cells isolated from a patient, or from cultured T-cell hybridomas. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and Li (2005) Nat Biotechnol. 23:349-354. Modified TCR comprising one or more heterologous antigen-binding domains (e.g. VH or a fragment thereof, or VL or a fragment thereof) and a native TCR (alpha or beta) constant domain are described in Int'l Patent Pub. No. WO 2019/157454, incorporated by reference herein in its entirety.

A “T cell receptor” or “TCR” refers to a molecule that contains a variable α and β chains (also known as TCRα and TCRβ, respectively) or a variable γ and δ chains (also known as TCRγ and TCRδ, respectively) and that is capable of specifically binding to an antigen peptide bound to a MHC receptor. For example, the TCR or its extracellular antigen-binding domain binds an antigen with a KD affinity of about 1×10⁻⁷ or less, about 5×10⁻⁸ or less, about 1×10⁻⁸ or less, about 5×10⁻⁹ or less, about 1×10⁻⁹ or less, about 5×10⁻¹⁰ or less, about 1×10⁻¹⁰ or less, about 5×10⁻¹¹ or less, about 1×10⁻¹¹ or less, about 5×10⁻¹² or less, or about 1×10⁻¹² or less. In some embodiments, the TCR is in the ap form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et ah, Immunobiology: The Immune System in Health and Disease, 3 rd Ed., Current Biology Publications, p. 4:33, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. The term “TCR” as used herein also refers to a TCR modified to include a VH and/or VL of an antibody. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) {see, e.g., Jores et al., Pwc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

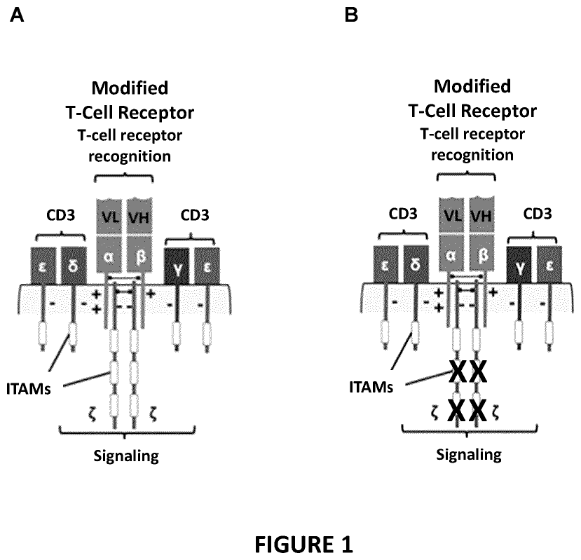

The modified TCR may comprise (a) an extracellular domain that comprises antigen-binding fragments, typically and antibody fragment (Examples of antibody fragments include but are not limited to Fv, Fab, Fab′, Fab′-SH, F(ab′)2; diabodies; linear antibodies; variable heavy chain (VH) regions, VHH antibodies, single-chain antibody molecules such as scFvs and single-domain antibodies; and multispecific antibodies formed from antibody fragments). notably CDRs of an antibody, such as all three CDRs of a heavy chain variable region (VH) and/or a light chain variable region (VL), and (b) a native or variant constant region of an alpha, beta, gamma or delta chain. The modified TCR may thus comprise one or more antigen-binding domain fused to one or both of the TRAC or TRBC or a fragment or variant thereof as described herein. As previously defined, the antigen binding domain may be a VH, a VL, a single domain antibody, such as a VHH or a nanobody, or an scFv or any multispecific antibody formed from antibody fragment as herein described. It is herein intended that in embodiments, wherein one antigen binding domain is bound to a TRAC (or fragment or variant thereof) and one antigen binding domain is bound to a TRBC (or fragment or variant thereof), both antigen binding domains may be the same or different. They may have the same specificity (binding the same antigen or the same epitope) or not. In some embodiments, the modified TCR may comprise one or more heterologous polypeptides, for example, (a) VH of an antibody or a fragment or variant having at least 60%, 65%, 70%, 75%, 80%, 85%, 90%, 95%, or 98% sequence identity thereto (and preferably comprising all three CDRs, or CDRs at least 60%, 65%, 70%, 75%, 80%, 85%, 90%, 95%, or 98% identical to the parental CDRs), fused to TRBC1 (SEQ ID NO: 5) or TRBC2 (SEQ ID NO: 6), or a fragment or variant of TRBC1 (SEQ ID NO: 5), TRBC2 (SEQ ID NO: 6) or a murinized version thereof (SEQ ID NO:28-29), having at least 90% sequence identity thereto, and (b) a VL of an antibody or a fragment or variant having at least at least 60%, 65%, 70%, 75%, 80%, 85%, 90%, 95%, or 98% sequence identity thereto (and preferably comprising all three CDRs, or CDRs at least 90% identical to the parental CDRs), fused to TRAC (SEQ ID NO: 4), a fragment or variant of TRAC (SEQ ID NO: 4), or a murinized version thereof (SEQ ID NO:30-31) having at least 60%, 65%, 70%, 75%, 80%, 85%, 90%, 95%, or 98% sequence identity thereto. See FIG. 1A. The modified TCR may optionally further comprise a native or variant CD3zeta polypeptide, e.g. a modified CD3zeta polypeptide (SEQ ID NO: 7) in which one or two of the ITAM domains (e.g. ITAM2 and ITAM3) have been deleted. See FIG. 1B.

In some designs, the HI-TCR comprises (a) a chimeric TCR alpha chain comprising an antigen binding domain or fragment thereof, such as a VH or fragment thereof fused to a native or variant TRAC or fragment thereof, optionally in which one to three amino acids of the VH (or TRAC) are removed, and (b) a chimeric TCR beta chain comprising an antigen binding domain or fragment thereof, such as a VL or fragment thereof fused to a native or variant TRBC or fragment thereof, optionally in which one to three amino acids of the VL (or TRBC) are removed. In other designs, the HI-TCR comprises (a) a chimeric TCR alpha chain comprising an antigen binding domain or fragment thereof such as a VL or fragment thereof fused to a native or variant TRAC or fragment thereof, optionally in which one to three amino acids of the VL (or TRAC) are removed, and (b) a chimeric TCR beta chain comprising an antigen binding domain or fragment thereof such as VH or fragment thereof fused to a native or variant TRBC or fragment thereof, optionally in which one to three amino acids of the VH (or TRBC) are removed. In yet other designs, the HI-TCR comprises just an antigen binding domain or fragment thereof, such as an scFv, a VHH, a VH or fragment thereof fused to a native or variant TRAC or fragment thereof, or fused to a native or variant TRBC or fragment thereof, optionally in which one to three amino acids of the VH (or TRAC or TRBC) are removed. HI-TCR (HIT-CAR) are described in Int'l Pat. Pub. No. WO 2019/157454, incorporated by reference herein in its entirety. Recombinant HLA-independent (or non-HLA restricted) modified TCR (referred to as “HI-TCRs”) that bind to an antigen of interest in an HLA-independent manner are described in International Application No. WO 2019/157454. Such HI-TCRs comprise an antigen binding chain that comprises: (a) a heterologous antigen-binding domain that binds to an antigen in an HLA-independent manner, for example, an antigen-binding fragment of an immunoglobulin variable region; and (b) a constant domain that is capable of associating with (and consequently activating) a CD3zeta polypeptide. Preferably, the antigen-binding domain or fragment thereof comprises: (i) a heavy chain variable region (VH) of an antibody and/or (ii) a light chain variable region (VL) of an antibody. The constant domain of the TCR is, for example, a native or modified TRAC polypeptide (SEQ ID NO: 4 or variant thereof), or a native or modified TRBC polypeptide (SEQ ID NO: 5 or 6 or variant thereof). The constant domain of the TCR is, for example, a native TCR constant domain (alpha or beta) or fragment thereof.

In certain embodiments, the extracellular antigen-binding domain comprises a heavy chain variable region (VH) and/or a light chain variable region (VL) of an antibody, wherein the VH or the VL is capable of dimerizing with another extracellular antigen binding domain comprising a VL or a VH (e.g., forming a fragment variable (Fv)). In certain embodiments, the Fv is a human Fv. In certain embodiments, the Fv is a humanized Fv. In certain embodiments, the Fv is a murine Fv. In certain embodiments, the Fv is identified by screening a Fv phage library with an antigen-Fc fusion protein.

Additional extracellular antigen-binding domains targeting an interested antigen can be obtained by sequencing an existing scFv or a Fab region of an existing antibody targeting the same antigen.

In certain embodiments, the dimerized extracellular antigen-binding domain of a presently disclosed HI-TCR is a murine Fv. In certain embodiments, the dimerized extracellular antigen-binding domain is an Fv that binds to a human tumor antigen as previously defined.

In certain embodiments, the extracellular antigen-binding domain is an Fv, and specifically binds to a human CD19 polypeptide (e.g., a human CD19 polypeptide).

In certain embodiments, the Fv comprises a heavy chain variable region (V_{H}) comprising the amino acid sequence set forth in SEQ ID NO: 36 or 40. In certain embodiments, the Fv comprises a light chain variable region (V_{L}) comprising the amino acid sequence set forth in SEQ ID NO: 37 or 40. In certain embodiments, the Fv comprises V_{H} comprising the amino acid sequence set forth in SEQ ID NO: 36 or 40 and a V_{L} comprising the amino acid sequence set forth in SEQ ID NO: 37 or 41. In certain embodiments, the extracellular antigen binding domain comprises a V_{H} comprising an amino acid sequence that is at least about 80% (e.g., at least about 85%, at least about 90%, or at least about 95%) homologous or identical to SEQ ID NO: 36 or 40. For example, the extracellular antigen-binding domain comprises a V_{H} comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to SEQ ID NO: 7. In certain embodiments, the extracellular antigen-binding domain comprises a V_{H} comprising the amino sequence set forth in SEQ ID NO: 37 or 40. In certain embodiments, the extracellular antigen-binding domain comprises a V_{L} comprising an amino acid sequence that is at least about 80% (e.g, at least about 85%, at least about 90%, or at least about 95%) homologous to SEQ ID NO: 37 or 41. For example, the extracellular antigen-binding domain comprises a V_{L} comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to SEQ ID NO: 37 or 41. In certain embodiments, the extracellular antigen-binding domain comprises a V_{L} comprising the amino acid sequence set forth in SEQ ID NO: 37 or 41. In certain embodiments, the extracellular antigen-binding domain comprises a V_{H} comprising an amino acid sequence that is at least about 80% (e.g., at least about 85%, at least about 90%, or at least about 95%) homologous to SEQ ID NO: 36 or 40, and a V_{L} comprising an amino acid sequence that is at least about 80% (e.g, at least about 85%, at least about 90%, or at least about 95%) homologous or identical to SEQ ID NO: 37 or 41 respectively. In certain embodiments, the extracellular antigen-binding domain comprises a V_{H} comprising the amino acid sequence set forth in SEQ ID NO: 36 or 40 and a V_{L} comprising the amino acid sequence set forth in SEQ ID NO: 37 or 41 respectively.

In certain embodiments, the extracellular antigen-binding domain is an Fv, and specifically binds to a human PSMA polypeptide (e.g., a human PSMA polypeptide).

In certain embodiments, the Fv comprises a heavy chain variable region (V_{H}) comprising the amino acid sequence set forth in SEQ ID NO: 44. In certain embodiments, the Fv comprises a light chain variable region (V_{L}) comprising the amino acid sequence set forth in SEQ ID NO: 45. In certain embodiments, the Fv comprises V_{H} comprising the amino acid sequence set forth in SEQ ID NO: 44 and a V_{L} comprising the amino acid sequence set forth in SEQ ID NO: 45. In certain embodiments, the extracellular antigen binding domain comprises a V_{H} comprising an amino acid sequence that is at least about 80% (e.g., at least about 85%, at least about 90%, or at least about 95%) homologous or identical to SEQ ID NO: 44. For example, the extracellular antigen-binding domain comprises a V_{H} comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to SEQ ID NO: 44. In certain embodiments, the extracellular antigen-binding domain comprises a V_{H} comprising the amino sequence set forth in SEQ ID NO: 44. In certain embodiments, the extracellular antigen-binding domain comprises a V_{L} comprising an amino acid sequence that is at least about 80% (e.g, at least about 85%, at least about 90%, or at least about 95%) homologous to SEQ ID NO: 45. For example, the extracellular antigen-binding domain comprises a V_{L} comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to SEQ ID NO: 45. In certain embodiments, the extracellular antigen-binding domain comprises a V_{L} comprising the amino acid sequence set forth in SEQ ID NO: 45. In certain embodiments, the extracellular antigen-binding domain comprises a V_{H} comprising an amino acid sequence that is at least about 80% (e.g., at least about 85%, at least about 90%, or at least about 95%) homologous to SEQ ID NO: 44, and a V_{L} comprising an amino acid sequence that is at least about 80% (e.g, at least about 85%, at least about 90%, or at least about 95%) homologous or identical to SEQ ID NO: 45 respectively. In certain embodiments, the extracellular antigen-binding domain comprises a V_{H} comprising the amino acid sequence set forth in SEQ ID NO: 44 and a V_{L} comprising the amino acid sequence set forth in SEQ ID NO: 45 respectively.

Unlike chimeric antigen receptors, which typically themselves comprise an intracellular signaling domain, the HI-TCR does not directly produce an activating signal; instead, the antigen-binding chain associates with and consequently activates a CD3zeta polypeptide (SEQ ID NO: 7). The immune cells comprising the recombinant TCR provide superior activity when the antigen has a low density on the cell surface of less than about 10,000 molecules per cell, e.g. less than about 5,000, 4,000, 3,000, 2,000, 1,000, 500, 250 or 100 molecules per cell. In some embodiments, the antigen is expressed at low density by the target cell, e.g., less than about 6,000 molecules of the target antigen per cell. In some embodiments, the antigen is expressed at a density of less than about 5,000 molecules, less than about 4,000 molecules, less than about 3,000 molecules, less than about 2,000 molecules, less than about 1,000 molecules, or less than about 500 molecules of the target antigen per cell. In some embodiments, the antigen is expressed at a density of less than about 2,000 molecules, such as e.g., less than about 1,800 molecules, less than about 1,600 molecules, less than about 1,400 molecules, less than about 1,200 molecules, less than about 1,000 molecules, less than about 800 molecules, less than about 600 molecules, less than about 400 molecules, less than about 200 molecules, or less than about 100 molecules of the target antigen per cell. In some embodiments, the antigen is expressed at a density of less than about 1,000 molecules, such as e.g., less than about 900 molecules, less than about 800 molecules, less than about 700 molecules, less than about 600 molecules, less than about 500 molecules, less than about 400 molecules, less than about 300 molecules, less than about 200 molecules, or less than about 100 molecules of the target antigen per cell. In some embodiments, the antigen is expressed at a density ranging from about 5,000 to about 100 molecules of the target antigen per cell, such as e.g., from about 5,000 to about 1,000 molecules, from about 4,000 to about 2,000 molecules, from about 3,000 to about 2,000 molecules, from about 4,000 to about 3,000 molecules, from about 3,000 to about 1,000 molecules, from about 2,000 to about 1,000 molecules, from about 1,000 to about 500 molecules, from about 500 to about 100 molecules of the target antigen per cell. In some embodiments, the recombinant TCR T cell therapy targets an antigen that is expressed at low density compared to a density in a wild-type cell.

The CD3zeta polypeptide optionally comprises an intracellular domain of a co-stimulatory molecule or a fragment thereof. Alternatively, the antigen binding domain optionally comprises a co-stimulatory domain that is capable of stimulating an immunoresponsive cell upon the binding of the antigen binding chain to the antigen. Example co-stimulatory domains include stimulatory domains, or fragments or variants thereof, from CD28 (SEQ ID NO: 8-9), 4-1BB (CD137) (SEQ ID NO: 10-11), ICOS (SEQ ID NO: 12), CD27, OX 40 (CD134) (SEQ ID NO: 13), DAP10, DAP12, 2B4, CD40, FCER1G or GITR (AITR). For T cells, CD28, CD27, 4-1BB (CD137), ICOS may be preferred. For NK cells, DAP10, DAP12, 2B4 may be preferred. Combinations of two co-stimulatory domains are contemplated, e.g. CD28 and 4-1BB, or CD28 and OX40.

The foregoing modified immune cell expressing an antigen-specific receptor, e.g. modified TCR, preferably comprises one or more further features as described herein: inactivation (e.g. mutation or inhibition) of the SUV39H1 gene, and/or inactivation of one or two ITAM domains of the CD3zeta intracellular signaling region of the antigen-specific receptor, and/or inactivation of one or both endogenous TCR chains (e.g. deletion or disruption of endogenous TCR-alpha and/or TCR-beta) and/or addition of a co-stimulatory receptor, or combinations of one, two, three or all of such features.

In some embodiments, the TCR chains contain a constant domain. For example, like immunoglobulins, the extracellular portion of TCR chains (e.g., α-chain, β-chain) can contain two immunoglobulin domains, a variable domain {e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5th ed.) at the N-terminus, and one constant domain (e.g., α-chain constant domain or Cα, typically amino acids 117 to 259 based on Kabat, or β-chain constant domain or Cβ, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contain a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3 gamma chain, a CD3 delta chain, two CD3 epsilon chains, and a homodimer of CD3zeta chains. The CD3 gamma, CD3delta, and CD3 epsilon chains are highly related cell surface proteins of the immunoglobulin superfamily containing a single immunoglobulin domain. The transmembrane regions of the CD3 gamma, CD3 delta, and CD3 epsilon chains are negatively charged, which is a characteristic that allows these chains to associate with the positively charged T cell receptor chains. The intracellular tails of the CD3 gamma, CD3 delta, and CD3 epsilon chains each contain a single conserved motif known as an immunoreceptor tyrosine-based activation motif or ITAM, whereas each CD3zeta chain has three. Generally, ITAMs are involved in the signaling capacity of the TCR complex. These accessory molecules have negatively charged transmembrane regions and play a role in propagating the signal from the TCR into the cell. The CD3 gamma-, delta-, epsilon- and zeta-chains, together with the TCR, form what is known as the T cell receptor complex.

In some embodiments, the TCR may be a heterodimer of two chains alpha and beta (optionally gamma and delta) or it may be a single chain TCR construct. In some embodiments, the TCR is a heterodimer containing two separate chains (alpha and beta chains or gamma and delta chains) that are linked, such as by a disulfide bond or disulfide bonds. Variants of TCRs are disclosed in Int'l Pat. Pub. No. WO 2018/067993, incorporated herein by reference in its entirety, and in Baeuerle, et al. Synthetic TRuC receptors engaging the complete T cell receptor for potent anti-tumor response. Nat Commun 10, 2087 (2019). For example, any one or more, or two or more, of the alpha, beta, gamma or epsilon chains may be fused to an antibody variable region, e.g., VH and/or VL such as an scFv.

Exemplary antigen-specific receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen-specific receptors include a CAR as described in U.S. Pat. No. 7,446,190, and those described in International Patent Application Publication No.: WO/2014055668 A1.

### Co-Stimulatory Ligands and Receptors

The cells of the disclosure with modified SUV39H1 expression may further comprise at least one or at least two exogenous co-stimulatory ligands.

In certain embodiments, the immune cell comprises an exogenous or a recombinant (e.g., the cell is transduced with at least one) co-stimulatory ligand. In certain embodiments, the immune cell co-expresses a CAR or an exogenous TCR (including modified TCR) and the at least one exogenous co-stimulatory ligand. The interaction between the CAR or the modified TCR and at least one exogenous co-stimulatory ligand provides a non-antigen-specific signal important for full activation of an immunoresponsive cell (e.g, T cell). Co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, but are not limited to, nerve growth factor (NGF), CD40L (CD40L)/CD154, CD137L/4-1BBL, TNF-α, CD134L/OX40L/CD252, CD27L/CD70, Fas ligand (FasL), CD30L/CD153, tumor necrosis factor beta (TNFP)/lymphotoxin-alpha (LTa), lymphotoxin-beta (TTb), CD257/B cell-activating factor (BAFF)/Blys/THANK/Tall-1, glucocorticoid-induced TNF Receptor ligand (GITRL), and TNF-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain (fold). Immunoglobulin superfamily ligands include, but are not limited to, CD80 and CD86, both ligands for CD28. In certain embodiments, the at least one co-stimulatory ligand is selected from the group consisting of 4-1BBL, CD275, CD80, CD86, CD70, OX40L, CD48, TNFRSF14, and combinations thereof. In certain embodiments, the immunoresponsive cell comprises or consists of one exogenous or recombinant co-stimulatory ligand. In certain embodiments, the one exogenous or recombinant co-stimulatory ligand is 4-1BBL or CD80. In certain embodiments, the one exogenous or recombinant co-stimulatory ligand is 4-1BBL. In certain embodiments, the immunoresponsive cell comprises or consists of two exogenous or recombinant co-stimulatory ligands. In certain embodiments, the two exogenous or recombinant co-stimulatory ligands are 4-1BBL and CD80.

In certain embodiments, the immunoresponsive cell can comprise or be transduced with at least one chimeric co-stimulatory receptor (CCR). As used herein, the term “chimeric co-stimulatory receptor” or “CCR” refers to a chimeric receptor that binds to an antigen, and, upon its binding to the antigen, provides a co-stimulatory signal to a cell (e.g., a T cell) comprising the CCR, but does not alone provide an activation signal to the cell. CCR is described in Krause, et al., J. Exp. Med. (1998); 188(4):619-626, and US20020018783, which is incorporated by reference in its entirety. CCRs mimic co-stimulatory signals, but unlike, CARs, do not provide a T-cell activation signal, e.g., CCRs lack a E03z polypeptide. CCRs provide co-stimulation, e.g., a CD284ike signal, in the absence of the natural co-stimulatory ligand on the antigen-presenting cell. A combinatorial antigen recognition, i.e., use of a CCR in combination with a CAR, can augment T-cell reactivity against the dual-antigen expressing T cells, thereby improving selective tumor targeting. See WO2014/055668, which is incorporated by reference in its entirety. Kloss et al., describe a strategy that integrates combinatorial antigen recognition, split signaling, and, critically, balanced strength of T-cell activation and co stimulation to generate T cells that eliminate target cells that express a combination of antigens while sparing cells that express each antigen individually (Kloss et al., Nature BioTechnology (2013); 3 I(I):7I-75, the content of which is incorporated by reference in its entirety). With this approach, T-cell activation requires CAR-mediated recognition of one antigen, whereas co-stimulation is independently mediated by a CCR specific for a second antigen. To achieve tumor selectivity, the combinatorial antigen recognition approach diminishes the efficiency of T-cell activation to a level where it is ineffective without rescue provided by simultaneous CCR recognition of the second antigen. In certain embodiments, the CCR comprises an extracellular antigen-binding domain that binds to a second antigen, a transmembrane domain, and a co-stimulatory signaling region that comprises at least one co-stimulatory molecule. In certain embodiments, the CCR does not alone deliver an activation signal to the cell. Non limiting examples of co-stimulatory molecules include CD28, 4-1BB, OX40, ICOS, DAP-10 and any combination thereof. In certain embodiments, the co-stimulatory signaling region of the CCR comprises one co-stimulatory signaling molecule. In certain embodiments, the one co-stimulatory signaling molecule is CD28. In certain embodiments, the one co-stimulatory signaling molecule is 4-1BB. In certain embodiments, the co-stimulatory signaling region of the CCR comprises two co stimulatory signaling molecules. In certain embodiments, the two co-stimulatory signaling molecules are CD28 and 4-1BB. A second antigen is selected so that expression of both the first antigen and the second antigen is restricted to the targeted cells (e.g., cancerous tissue or cancerous cells). Similar to a CAR, the extracellular antigen-binding domain can be a scFv, a Fab, a F(ab)2, or a fusion protein with a heterologous sequence to form the extracellular antigen-binding domain. In certain embodiments, the CCR is co-expressed with a CAR or a modified TCR binding to an antigen that is different from the antigen to which the CCR binds, e.g., the CAR or the modified TCR binds to a first antigen and the CCR binds to a second antigen.

Example co-stimulatory ligands, molecules and receptors (or fusion polypeptides) are described in Int'l Pat. Pub. No. WO-2021/016174, incorporated by reference herein in its entirety. Illustrative booster sequences include SEQ ID NO: 32-33 and 53-54.

### Antigens

Among the antigens targeted by the antigen-specific receptors are those expressed in the context of a disease, condition, or cell type to be targeted via the adoptive cell therapy. Among the diseases and conditions are proliferative, neoplastic, and malignant diseases and disorders, more particularly cancers. Infectious diseases and autoimmune, inflammatory or allergic diseases are also contemplated.

The cancer may be a solid cancer or a “liquid tumor” such as cancers affecting the blood, bone marrow and lymphoid system, also known as tumors of the hematopoietic and lymphoid tissues, which notably include leukemia and lymphoma. Liquid tumors include for example acute myelogenous leukemia (AML), chronic myelogenous leukemia (CML), acute lymphocytic leukemia (ALL), and chronic lymphocytic leukemia (CLL), (including various lymphomas such as mantle cell lymphoma, non-Hodgkins lymphoma (NHL), adenoma, squamous cell carcinoma, laryngeal carcinoma, gallbladder and bile duct cancers, cancers of the retina such as retinoblastoma).

Solid cancers notably include cancers affecting one of the organs selected from the group consisting of colon, rectum, skin, endometrium, lung (including non-small cell lung carcinoma), uterus, bones (such as Osteosarcoma, Chondrosarcomas, Ewing's sarcoma, Fibrosarcomas, Giant cell tumors, Adamantinomas, and Chordomas), liver, kidney, esophagus, stomach, bladder, pancreas, cervix, brain (such as Meningiomas, Glioblastomas, Lower-Grade Astrocytomas, Oligodendrocytomas, Pituitary Tumors, Schwannomas, and Metastatic brain cancers), ovary, breast, head and neck region, testis, prostate and the thyroid gland.

Preferably, a cancer according to the disclosure is a cancer affecting the blood, bone marrow and lymphoid system as described above. In some embodiments, the cancer is, or is associated, with multiple myeloma.

Diseases according to the disclosure also encompass infectious diseases or conditions, such as, but not limited to, viral, retroviral, bacterial, and protozoal infections, HIV immunodeficiency, Cytomegalovirus (CMV), Epstein-Barr virus (EBV), adenovirus, BK polyomavirus.

Diseases according to the disclosure also encompass autoimmune or inflammatory diseases or conditions, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease multiple sclerosis, asthma, and/or diseases or conditions associated with transplant. In such circumstances, a T-regulatory cell may be the cell in which SUV39H1 is knocked out.

In some embodiments, the antigen is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells. In some such embodiments, a multi-targeting and/or gene disruption approach as provided herein is used to improve specificity and/or efficacy. Among the antigens targeted by the antigen-specific receptors in the context of proliferative, neoplastic, and malignant diseases and disorders, more particularly cancers, any tumor antigen (antigenic peptide) can be used. Sources of antigen include, but are not limited to, cancer proteins. The antigen can be expressed as a peptide or as an intact protein or portion thereof. The intact protein or a portion thereof can be native or mutagenized.

In some embodiments, the antigen is a universal tumor antigen. The term “universal tumor antigen” refers to an immunogenic molecule, such as a protein, that is, generally, expressed at a higher level in tumor cells than in non-tumor cells and also is expressed in tumors of different origins. In some embodiments, the universal tumor antigen is expressed in more than 30%, 40%, 50%, 60%, 70%, 75%, 80%, 85%, 90% or more of human cancers. In some embodiments, the universal tumor antigen is expressed in at least three, at least four, at least five, at least six, at least seven, at least eight or more different types of tumors. In some cases, the universal tumor antigen may be expressed in non-tumor cells, such as normal cells, but at lower levels than it is expressed in tumor cells. In some cases, the universal tumor antigen is not expressed at all in non-tumor cells, such as not expressed in normal cells. Exemplary universal tumor antigens include, for example, human telomerase reverse transcriptase (hTERT), survivin, mouse double minute 2 homolog (MDM2), cytochrome P450 11B1 (CYP1B), HER2/neu, p95HER2, Wilms' tumor gene 1 (WT1), livin, alphafetoprotein (AFP), carcinoembryonic antigen (CEA), mucin 16 (MUC16), MUC1, prostate-specific membrane antigen (PSMA), p53 or cyclin (DI).

In some aspects, the antigen is expressed on multiple myeloma, such as CD38, CD138, and/or CS-1. Other exemplary multiple myeloma antigens include CD56, TIM-3, CD33, CD123, and/or CD44. Antibodies or antigen-binding fragments directed against such antigens are known and include, for example, those described in U.S. Pat. Nos. 8,153,765; 8,603,477, 8,008,450; U.S. published application No. US20120189622; and published international PCT application Nos. WO2006099875, WO2009080829 or WO2012092612. In some embodiments, such antibodies or antigen-binding fragments thereof (e.g. scFv) can be used to generate a CAR or a modified TCR (Hi-TCR-

In some embodiments, the antigen may be one that is expressed or upregulated on cancer or tumor cells, but that also may be expressed in an immune cell, such as a resting or activated T cell. For example, in some cases, expression of hTERT, survivin and other universal tumor antigens are reported to be present in lymphocytes, including activated T lymphocytes (see e.g., Weng et al. (1996) J Exp. Med., 183:2471-2479; Hathcock et al. (1998) J Immunol, 160:5702-5706; Liu et al. (1999) Proc. Natl Acad Sci., 96:5147-5152; Turksma et al. (2013) Journal of Translational Medicine, 11: 152). Likewise, in some cases, CD38 and other tumor antigens also can be expressed in immune cells, such as T cells, such as upregulated in activated T cells. For example, in some aspects, CD38 is a known T cell activation marker.

In some embodiments, the cancer is, or is associated, with overexpression of HER2 or p95HER2. p95HER2 is a constitutively active C-terminal fragment of HER2 that is produced by an alternative initiation of translation at methionine 611 of the transcript encoding the full-length HER2 receptor. The amino acid sequence of the p95HER2 extracellular domain is

MPIWKFPDEEGACQPCPINCTHSCVDKDDKGCPAEQRASPLT.

HER2 or p95HER2 has been reported to be overexpressed in breast cancer, as well as gastric (stomach) cancer, gastroesophageal cancer, esophageal cancer, ovarian cancer, uterine endometrial cancer, cervix cancer, colon cancer, bladder cancer, lung cancer, and head and neck cancers. Patients with cancers that express the p95HER2 fragment have a greater probability of developing metastasis and a worse prognosis than those patients who mainly express the complete form of HER2. Saez et al., Clinical Cancer Research, 12:424-431 (2006).

Antibodies that can specifically bind p95HER2 compared to HER2 (i.e., bind p95HER2 but do not bind significantly to full length HER2 receptor) are disclosed in Sperinde et al., Clin. Cancer Res. 16, 4226-4235 (2010) and U.S. Patent Pub. No. 2013/0316380, incorporated by reference herein in their entireties. Hybridomas that produce monoclonal antibodies that can specifically bind p95HER2 compared to HER2 are disclosed in Int'l. Patent Pub. No. WO/2010/000565, and in Parra-Palau et al., Cancer Res. 70, 8537-8546 (2010). An example CAR binds the epitope PIWKFPD of p95HER2 with a binding affinity KD of 10⁻⁷ M or less, 10⁻⁸ M or less, 10⁻⁹ M or less or 10⁻¹⁰ M or less. In some embodiments, a CAR or a modified TCR (e.g. Hi-TCR) as herein described comprises VH/VL sequences as described in WO2021239965.

The modified immune cells, compositions and methods disclosed herein include cells in which, optionally, the SUV39H1 gene is inactivated and that express a modified TCR such as a Hi-TCR as herein disclosed and/or a chimeric antigen receptor (CAR) comprising: a) an extracellular antigen-binding domain that specifically binds a target antigen, e.g. a binding affinity of about 10⁻⁷ M or less, or about 10⁻⁸ M or less, or about 10⁻⁹ M or less or about 10⁻¹⁰ M or less, b) a transmembrane domain, c) optionally one or more costimulatory domains, and d) an intracellular signaling domain comprising a modified CD3zeta intracellular signaling domain that retains a single active ITAM. This can be accomplished by any means known in the art, e.g., ITAM2 and ITAM3 have been inactivated, or ITAM1 and ITAM2 have been inactivated. For example, a modified CD3zeta polypeptide retains only ITAM1 and the remaining CD3zeta domain is deleted (residues 90-164). As another example, ITAM1 is substituted with the amino acid sequence of ITAM3, and the remaining CD3zeta domain is deleted (residues 90-164).

Rius Ruiz et al., Sci. Transl. Med. 10, eaat1445 (2018) and U.S. Patent Pub. No. 2018/0118849, incorporated by reference herein in their entireties, describe a T-cell bispecific antibody that specifically binds to the epitope PIWKFPD of p95HER2 and to the CD3 epsilon chain of the TCR. The antibody designated p95HER2-TCB consists of an asymmetric two-armed immunoglobulin G1 (IgG1) that binds monovalently to CD3 epsilon and bivalently to p95HER2. The bispecific antibody has monovalent low affinity for CD3 epsilon of about 70 to 100 nM which reduces the chances of nonspecific activation, and a higher bivalent affinity for p95HER2 of about 9 nM.

When the antigen-specific receptor specifically binds p95HER2, the disclosure provides for a modified immune cell that is further modified so that it secretes a soluble (non-membrane-bound) bispecific antibody, e.g. BiTE (bispecific soluble antibody), that binds to both HER2 and a T cell activation antigen, e.g. CD3 epsilon or the constant chain (alpha or beta) of a TCR. Expressing the bispecific antibody may treat heterogeneous tumors that express both p95HER and HER2, and/or may mitigate effects of potential tumor cell escape through p95HER2 antigen loss following treatment with CAR-T cells targeting p95HER2. See, e.g., Choi et al., “CAR-T cells secreting BiTEs circumvent antigen escape without detectable toxicity,” Nature Biotechnology, 37:1049-1058 (2019).

In some embodiments as provided herein, an immune cell, such as a T cell, can be engineered to repress or disrupt the gene encoding the antigen in the immune cell so that the expressed antigen-specific receptor does not specifically bind the antigen in the context of its expression on the immune cell itself. Thus, in some aspects, this may avoid off-target effects, such as binding of the engineered immune cells to themselves, which may reduce the efficacy of the engineered in the immune cells, for example, in connection with adoptive cell therapy.

In some embodiments, such as in the case of an inhibitory CAR, the target is an off-target marker, such as an antigen not expressed on the diseased cell or cell to be targeted, but that is expressed on a normal or non-diseased cell which also expresses a disease-specific target being targeted by an activating or stimulatory receptor in the same engineered cell. Exemplary such antigens are MHC molecules, such as MHC class I molecules, for example, in connection with treating diseases or conditions in which such molecules become downregulated but remain expressed in non-targeted cells.

In some embodiments, the engineered immune cells can contain an antigen-specific receptor (e.g. CAR and/or Hi-TCR) that targets one or more other antigens. In some embodiments, the one or more other antigens is a tumor antigen or cancer marker. Other antigen targeted by antigen-specific receptors on the provided immune cells can, in some embodiments, include orphan tyrosine kinase receptor ROR1, tEGFR, Her2, p95HER2, LI-CAM, CD19, CD20, CD22, mesothelin, CEA, Claudin 18.2, and hepatitis B surface antigen, anti-folate receptor, CD23, CD24, CD30, CD33, CD38, CD44, EGFR, EGP-2, EGP-4, EPHa2, ErbB2, 3, or 4, FBP, FcRH5, fetal acethycholine e receptor, GD2, GD3, HMW-MAA, IL-22R-alpha, IL-13R-alpha2, kdr, kappa light chain, Lewis Y, LI-cell adhesion molecule, MAGE-A1, mesothelin, MUC1, MUC16, PSCA, NKG2D Ligands, NY-ESO-1, MART-1, gplOO, oncofetal antigen, ROR1, TAG72, VEGF-R2, carcinoembryonic antigen (CEA), prostate specific antigen, PSMA, Her2/neu, p95HER2, estrogen receptor, progesterone receptor, ephrinB2, CD 123, CS-1, c-Met, GD-2, and MAGE A3, CE7, Wilms Tumor 1 (WT-1), a cyclin, such as cyclin AI (CCNA1), and/or biotinylated molecules, and/or molecules expressed by HIV, HCV, HBV or other pathogens.

In some embodiments the extracellular antigen-binding domain binds to an e-antigen from the grey or dark genome. In some embodiments, the extracellular antigen binding domain binds to any of the tumor neoantigenic peptides disclosed in Int'l Pat. Pub. No. WO 2021/043804, incorporated by reference herein in its entirety. For example, the antigen-binding domain binds to any of the peptides of SEQ ID NO: 1-117 or to a neoantigenic peptide comprising at least 8, 9, 10, 11 or 12 amino acids that is encoded by a part of an open reading frame (ORF) of any of the fusion transcript sequences of any one of SEQ ID NO: 118-17492 of WO 2021/043804 or described in any of WO 2018/234367, WO 2022/189620, WO 2022/189626, and WO-2022/189639. In some embodiments, the antigen-specific receptor binds a pathogen-specific antigen. In some embodiments, the antigen-specific receptor is specific for viral antigens (such as HIV, HCV, HBV, etc.), bacterial antigens, and/or parasitic antigens.

Non-limiting examples of viruses include, Retroviridae (e.g. human immunodeficiency viruses, such as HIV-1 (also referred to as HDTV-III, LAVE or HTLV-Ill/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Fdoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=intemally transmitted; class 2=parenterally transmitted (i.e. Hepatitis C); Norwalk and related viruses, and astroviruses).

Non-limiting examples of bacteria include Pasteur ella, Staphylococci, Streptococcus, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, Corynebacterium diphtheriae, Corynebacterium sp., Erysipelothrix rhusiopathiae, Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema palladium, Treponema pertenue, Leptospira, Rickettsia, and Actinomyces israelii.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

In some embodiments, the antigen is an MHC restricted antigen. Peptide epitopes of tumor antigens, including universal tumor antigens, or pathogen antigens as described above are known in the art and, in some aspects, can be used to generate MHC-restricted antigen antibody or antibody fragments (see e.g. published PCT application No. WO2011009173 or WO2012135854 and published U.S. application No. US20140065708, see also Maus M V, Plotkin J, Jakka G, Stewart-Jones G, Riviere 1, Merghoub T, Wolchok J, Renner C, Sadelain M. An MHC-restricted antibody-based chimeric antigen receptor requires TCR-like affinity to maintain antigen specificity. Mol Ther Oncolytics. 2017 Jan. 11; 3:1-9; as well as Denkberg G, Reiter Y. Recombinant antibodies with T-cell receptor-like specificity: novel tools to study MHC class I presentation. Autoimmun Rev. 2006; 5:252-257; and Hulsmeyer M, Chames P, Hillig R C, Stanfield R L, Held G, Coulie P G. A major histocompatibility complex-peptide-restricted antibody and T cell receptor molecules recognize their target by distinct binding modes: crystal structure of human leukocyte antigen (HLA)-A1-MAGE-A1 in complex with FAB-HYB3. J Biol Chem. 2005; 280:2972-2980).

In some embodiments, the cell of the disclosure is genetically engineered to express two or more antigen-specific receptors on the cell, each recognizing a different antigen and typically each including a different intracellular signaling component. Such multi-targeting strategies are described, for example, in International Patent Application, Publication No.: WO 2014055668 AI (describing combinations of activating and costimulatory CARs, e.g., targeting two different antigens present individually on off-target, e.g., normal cells, but present together only on cells of the disease or condition to be treated) and Fedorov et al., Sci. Transl. Medicine, 5(215) (December, 2013) (describing cells expressing an activating and an inhibitory CAR, such as those in which the activating CAR binds to one antigen expressed on both normal or non-diseased cells and cells of the disease or condition to be treated, and the inhibitory CAR binds to another antigen expressed only on the normal cells or cells which it is not desired to treat).

Example antibodies include bispecific antibodies that are T-cell activating antibodies which bind not only the desired antigen but also an activating T-cell antigen such as CD3 epsilon or the constant chain (alpha or beta) of a TCR.

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive cell therapy. For example, in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-1 TK) gene (Wigler et al., Cell II:223, 1977) which confers ganciclovir sensitivity; the cellular hypoxanthine phosphribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In other embodiments of the disclosure, the cells, e.g., T cells, are not engineered to express recombinant antigen-specific receptors, but rather include naturally occurring antigen-specific receptors specific for desired antigens, such as tumor-infiltrating lymphocytes and/or T cells cultured in vitro or ex vivo, e.g., during the incubation step(s), to promote expansion of cells having particular antigen specificity. For example, in some embodiments, the cells are produced for adoptive cell therapy by isolation of tumor-specific T cells, e.g. autologous tumor infiltrating lymphocytes (TIL). The direct targeting of human tumors using autologous tumor infiltrating lymphocytes can in some cases mediate tumor regression (see Rosenberg S A, et al. (1988) N Engl J Med. 319: 1676-1680). In some embodiments, lymphocytes are extracted from resected tumors. In some embodiments, such lymphocytes are expanded in vitro. In some embodiments, such lymphocytes are cultured with lymphokines (e.g., IL-2). In some embodiments, such lymphocytes mediate specific lysis of autologous tumor cells but not allogeneic tumor or autologous normal cells.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

### Expression Cassettes, Vectors and Targeting Constructs

In some aspects, the genetic engineering involves introduction of a nucleic acid encoding the genetically engineered component or other component for introduction into the cell, such as a component encoding a gene-disruption protein or nucleic acid.

Generally, the engineering of CARs into immune cells (e.g., T cells) requires that the cells be cultured to allow for transduction and expansion. The transduction may utilize a variety of methods, but stable gene transfer is required to enable sustained CAR expression in clonally expanding and persisting engineered cells.

In some embodiments, gene transfer is accomplished by first stimulating cell growth, e.g., T cell growth, proliferation, and/or activation, followed by transduction of the activated cells, and expansion in culture to numbers sufficient for clinical applications.

Traditional techniques have utilized a suitable expression vector, in which case the immune cells are transduced with an expression cassette comprising a transgene, for example, an exogenous nucleic acid encoding a CAR or a modified TCR. Typically, the transgene or the expression cassette is cloned into a targeting construct, which provides for targeted integration of the expression cassette or the transgene at a targeted site within the genome (e.g., at TCR or at a SUV39H1 locus).

Any suitable targeting construct suitable for expression in a cell of the invention, particularly an immune cell, can be employed. In particular embodiments, the targeting construct is compatible for use with a homologous recombination system suitable for targeted integration of the nucleic acid sequence (transgene) at a site (e.g., a SUV39H1 locus) within the genome of the cell.

Known vectors include viral vectors and pseudotyped viral vectors, such as retrovirus (e.g., moloney murine leukemia virus, harvey murine sarcoma virus, murine mammary tumor virus, and rous sarcoma virus), lentivirus, adenovirus, adeno-associated virus (AAV), alphavirus, vaccinia virus, poxvirus, SV40-type viruses, polyoma viruses, Epstein-Barr viruses, herpes simplex virus, papilloma virus, polio virus, foamivirus, or Semliki Forest virus vectors; or transposase systems, such as Sleeping Beauty transposase vectors (see, e.g., Koste et al. (2014) Gene Therapy 2014 Apr. 3.; Carlens et al. (2000) Exp Hematol 28(10): 1137-46; Alonso-Camino et al. (2013) Mol Ther Nucl Acids 2, e93; Park et al., Trends Biotechnol. 2011 November; 29(11): 550-557). A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3: 102-109.

Methods of lentiviral transduction are also known. Exemplary methods are described in, e.g., Wang et al. (2012) J. Immunother. 35(9): 689-701; Cooper et al. (2003) Blood. 101: 1637-1644; Verhoeyen et al. (2009) Methods Mol Biol. 506: 97-114; and Cavalieri et al. (2003) Blood. 102(2): 497-505.

Non-viral systems for delivery of naked plasmids to cells include lipofection, nucleofection, microinjection, biolistics, virosomes, lipids, cationic lipid complexes, liposomes, immunoliposomes, nanoparticle, gold particle, or polymer complex, poly-lysine conjugates, synthetic polyamino polymers, other agent-enhanced uptake of DNA, and artificial viral envelopes or virions.

In some embodiments, recombinant nucleic acids are transferred into T cells via electroporation {see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Particularly useful vectors for generating a target construct that provides transgene vectorization for homologous recombination-mediated targeting include, but are not limited to, recombinant Adeno-Associated Virus (rAAV), recombinant non-integrating lentivirus (rNILV), recombinant non-integrating gamma-retrovirus (rNIgRV), single-stranded DNA (linear or circular), and the like. Such vectors can be used to introduce a transgene into an immune cell of the invention by making a targeting construct (see, for example, Miller, Hum. Gene Ther. 1(1):5-14 (1990); Friedman, Science 244: 1275-1281 (1989); Eglitis et al., BioTechniques 6:608-614 (1988); Tolstoshev et al., Current Opin. Biotechnol. 1:55-61 (1990); Sharp, Lancet 337: 1277-1278 (1991); Cornetta et al., Prog. Nucleic Acid Res. Mol. Biol. 36:311-322 (1989); Anderson, Science 226:401-409 (1984); Moen, Blood Cells 17:407-416 (1991); Miller et al., Biotechnology 7:980-990 (1989); Le Gal La Salle et al., Science 259:988-990 (1993); and Johnson, Chest 107:77S-83S (1995); Rosenberg et al., N. Engl. J. Med. 323:370 (1990); Anderson et al., U.S. Pat. No. 5,399,346; Scholler et al., Sci. Transl. Med. 4: 132-153 (2012; Parente-Pereira et al., J. Biol. Methods 1(2):e7 (1-9)(2014); Lamers et al., Blood 117(1):72-82 (2011); Reviere et al., Proc. Natl. Acad. Sci. USA 92:6733-6737 (1995); Wang et al., Gene Therapy 15: 1454-1459 (2008)).

In some embodiments, the exogenous nucleic acid or the targeting construct comprises a 5′ homology arm and a 3′ homology arm to promote recombination of the nucleic acid sequence into the cell genome at the nuclease cleavage site.

In some embodiments, an exogenous nucleic acid can be introduced into the cell using a single-stranded DNA template. The single-stranded DNA can comprise the exogenous nucleic acid and, in preferred embodiments, can comprise 5′ and 3′ homology arms to promote insertion of the nucleic acid sequence into the nuclease cleavage site by homologous recombination. The single-stranded DNA can further comprise a 5′ AAV inverted terminal repeat (ITR) sequence 5′ upstream of the 5′ homology arm, and a 3′ AAV ITR sequence 3′ downstream of the 3′ homology arm. In other particular embodiments, the targeting construct comprises in 5′ to 3′ order: a first viral sequence, a left homology arm, a nucleic acid sequence encoding an element that create polycistronic expression cassette (e.g. various viral and non-viral Internal Ribosome Entry Sites (IRES, e.g., FGF-I IRES, FGF-2 IRES, VEGF IRES, IGF-II IRES, NF-kB IRES, RUNX1 IRES, p53 IRES, hepatitis A IRES, hepatitis C IRES, pestivirus IRES, aphthovirus IRES, picomavirus IRES, poliovirus IRES and encephalomyocarditis virus IRES) and cleavable linkers (e.g, 2 A peptides, e.g., P2A, T2A, E2A and F2A peptides), preferably a cleavable linker), a transgene, a polyadenylation sequence, a right homology arm and a second viral sequence. In a preferred embodiment, the targeting construct comprises in 5′ to 3′ order: a first viral sequence, a left homology arm, a nucleic acid sequence encoding a self-cleaving linker (such as the porcine teschovirus 2A), a nucleic acid sequence encoding a CAR or a modified TCR (e.g. a Hi-CTR), a polyadenylation sequence, a right homology arm and a second viral sequence. Another suitable targeting construct can comprise sequences from an integrative-deficient Lentivirus (see, for example, Wanisch et al., Mol. Ther. 17(8): 1316-1332 (2009)).

In some embodiments, the viral nucleic acid sequence comprises sequences of an integrative-deficient Lentivirus. It is understood that any suitable targeting construction compatible with a homologous recombination system employed can be utilized. The AAV nucleic acid sequences that function as part of a targeting construct can be packaged in several natural or recombinant AAV capsids or particles. In a particular embodiment, the AAV particle is AAV6. In a particular embodiment, an AAV2-based targeting construct is delivered to the target cell using AAV6 viral particles. In a particular embodiment, the AAV sequences are AAV2, AAV5 or AAV6 sequences.

In some embodiments, the gene encoding an exogenous nucleic acid sequence of the invention can be introduced into the cell by transfection with a linearized DNA template. In some examples, a plasmid DNA encoding an exogenous nucleic acid sequence can include nuclease cleavage site (such as class II, type II, V or VI Cas nuclease) at both sides of the left homology arm such that the circular plasmid DNA is linearized and allows precise in-frame integration of exogenous DNA without backbone vector sequences (see for example Hisano Y, Sakuma T, Nakade S, et al. Precise in-frame integration of exogenous DNA mediated by CRISPR/Cas9 system in zebrafish. Sci Rep. 2015; 5:8841).

In some embodiments, the vector incorporates an endogenous promoter such as a TCR promoter. Such a vector could provide for expression in a manner similar to that provided by an endogenous promoter, such as a TCR promoter. Such a vector can be useful, for example, if the site of integration does not provide for efficient expression of a transgene, or if disruption of the endogenous gene controlled by the endogenous promoter would be detrimental to the T cell or would result in a decrease in its effectiveness in T cell therapy. In a preferred embodiment, such a vector can be useful, for example, if the site of integration does not provide for efficient expression of nucleic acid sequence encoding a CAR or a modified TCR. The promoter can be an inducible promoter or a constitutive promoter. Expression of a nucleic acid sequence under the control of an endogenous or vector-associated promoter occurs under suitable conditions for the cell to express the nucleic acid, for example, growth conditions, or in the presence of an inducer with an inducible promoter, and the like. Such conditions are well understood by those skilled in the art.

The targeting construct can optionally be designed to include an element that create polycistronic expression cassette (including but not limited to, various viral and non-viral Internal Ribosome Entry Sites (IRES, e.g., FGF-I IRES, FGF-2 IRES, VEGF IRES, IGF-II IRES, NF-kB IRES, RUNX1 IRES, p53 IRES, hepatitis A IRES, hepatitis C IRES, pestivirus IRES, aphthovirus IRES, picomavirus IRES, poliovirus IRES and encephalomyocarditis virus IRES) and cleavable linkers (e.g, 2 A peptides, e.g., P2A, T2A, E2A and F2A peptides)) directly upstream of the nucleic acid sequences encoding the transgene. In preferred embodiments, the targeting construct can optionally be designed to include a cleavable linked (e.g.: P2A, T2A, etc.) sequence directly upstream of the nucleic acid sequences encoding a therapeutic protein (e.g. an engineered antigen receptor). P2A and T2A are self-cleaving peptide sequences, which can be used for bicistronic or multicistronic expression of protein sequences (see Szymczak et al., Expert Opin. Biol. Therapy 5(5):627-638 (2005)).

Well-suited AAV constructs for HIT expressing in an immunoresponsive cell according to the present application are for example described in Mansilla-Soto, J., Eyquem, J., Haubner, S. et aL. HLA-independent T cell receptors for targeting tumors with low antigen density. Nat Med 28, 345-352 (2022) and have been used in the results included herein, notably for the in vivo experiments.

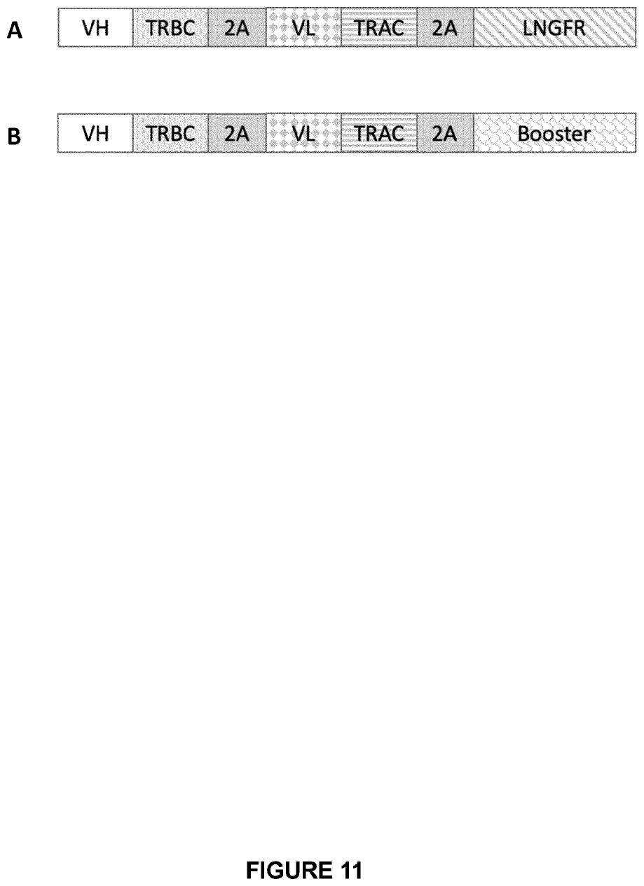

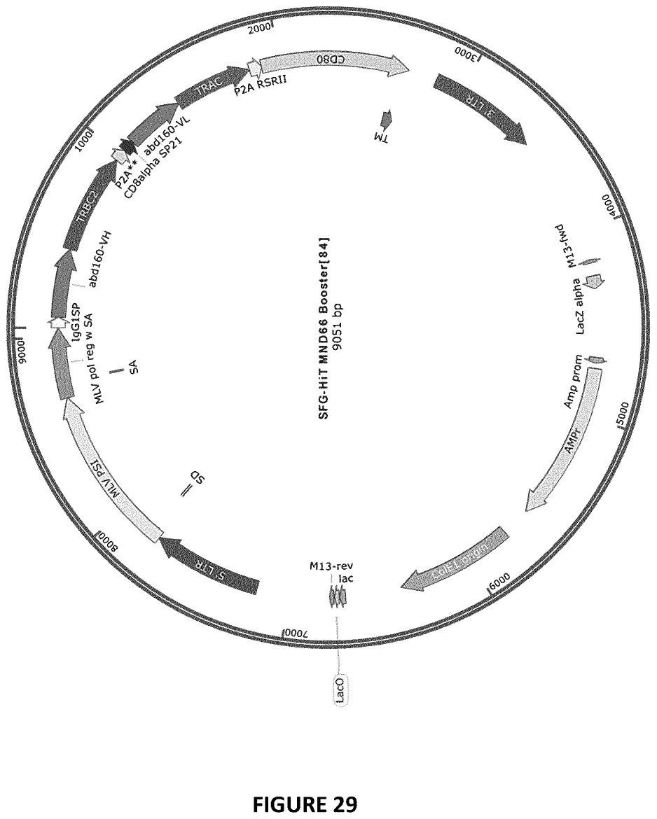

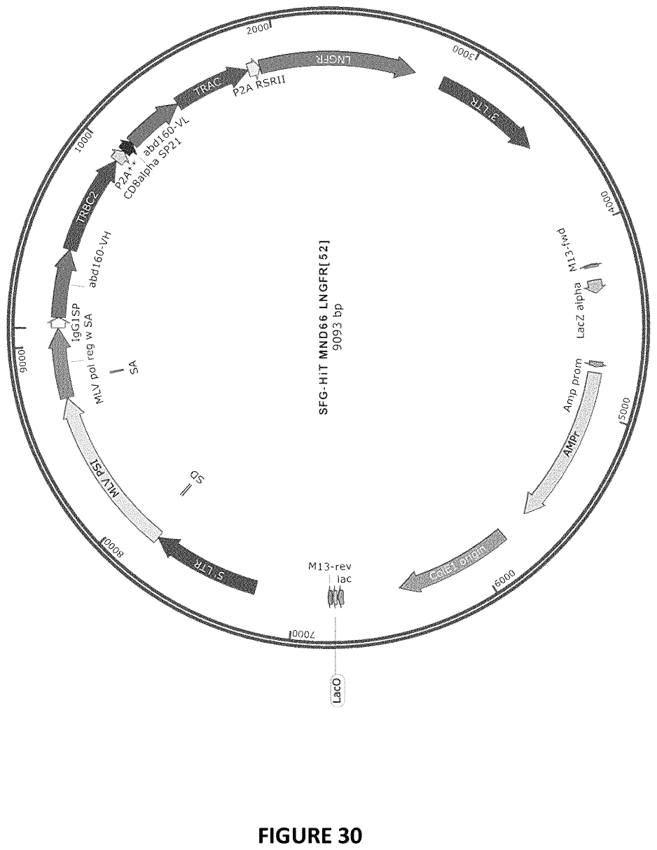

Typical well-suited constructs typically include a TRBC or TRAC sequence (which can be a native or modified TRBC or TRAC sequence, including murine sequences as described herein), a cleavable linker sequence (as defined above, but such as a 2A sequence), a TRAC or TRBC sequence (which can be a native or modified TRBC or TRAC sequence, including murine sequences as described herein). The TRBC and/or the TRAC sequence is typically fused (preferably in 5′) to a sequence coding for an antibody fragment as above described (e.g. a VH, a VH, an scFv, a single domain antibody, a VHH, etc.). In some embodiment, the booster (co-stimulatory ligand) sequence is included in the construct, such that in preferred embodiments, the construct further includes a cleavable linker sequence (e.g. a 2A sequence) and a booster (co-stimulatory ligand) sequence. Typically, the TRAC or TRBC sequence in the 3′ end of the construct is fused to cleavable linker which is also fused to the booster (co-stimulatory ligand and or costimulatory receptor CCR) sequence (see FIG. 11, as well as FIGS. 29-30). The booster ((co-stimulatory ligand and/or costimulatory receptor CCR) sequence can be any one as herein described and can be notably a CD80 sequence or a CD80_4-1BB sequence as herein described (see for example SEQ ID NO:32-33 and 52-53).

If desired, the targeting construct can optionally be designed to include a reporter, for example, a reporter protein that provides for identification of transduced cells. Exemplary reporter proteins include, but are not limited to, fluorescent proteins, such as mCherry, green fluorescent protein (GFP), blue fluorescent protein, for example, EBFP, EBFP2, Azurite, and mKalamal, cyan fluorescent protein, for example, ECFP, Cerulean, and CyPet, and yellow fluorescent protein, for example, YFP, Citrine, Venus, and YPet. Typically, the targeting construct comprises a polyadenylation (poly A) sequence 3′ of the transgene. In a preferred embodiment, the targeting construct comprises a polyadenylation (poly A) sequence in 3′ of the nucleic acid sequences encoding a CAR and/or a modified TCR (e.g. a Hi)-TCR).

### Method for Obtaining Cells According to the Disclosure

In certain non-limiting embodiments, an HI-TCR, a costimulatory ligand, a CCR or any other molecule/transgene disclosed herein is expressed by an immunoresponsive cell through a modified genomic locus. In certain embodiments, an expression cassette of the transgene is integrated into a targeted genomic locus of an immunoresponsive cell through targeted genome editing methods. In certain embodiments, the targeted genomic locus can be SUV39H1, CD3S, CD3e, CD247, B2M, TRAC, TRBC1, TRBC2, TRGC1 and/or TRGC2 loci.

Any suitable genetic editing methods and systems can be used to modify an endogenous T cell receptor locus. The genome editing methods disclosed herein can be used to modify the endogenous T cell receptor locus. In certain embodiments, a CRISPR system is used to modify T cell receptor locus. In certain embodiments, the CRISPR system targets exon 1 of a human TRAC locus. In certain embodiments, the CRISPR system comprises a guide RNA (gRNA) that targets exon 1 of a human TRAC locus.

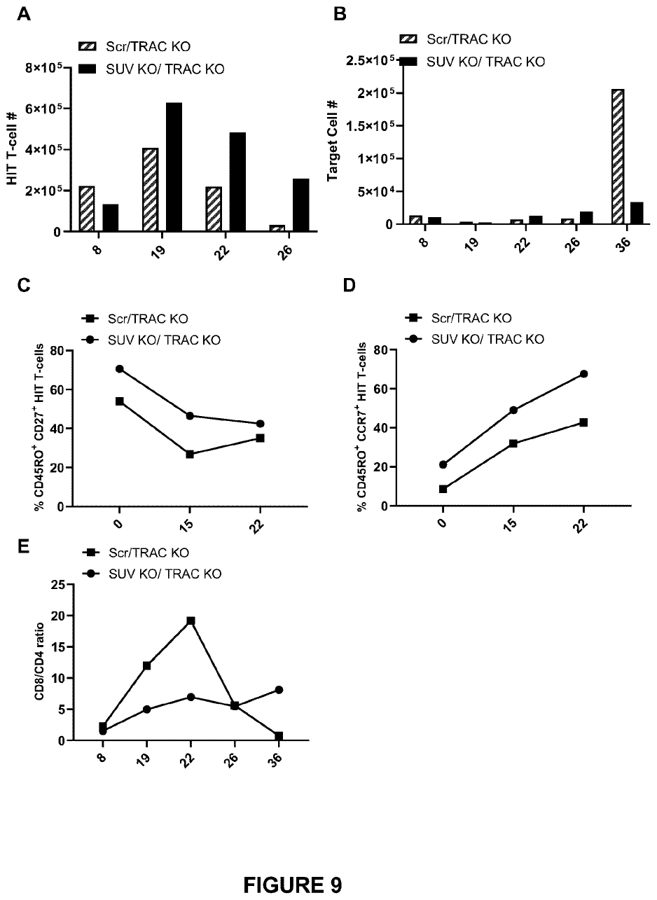

Typically, integration (knockin) of the transgene for example coding for an antigen receptor as herein defined (e.g., Hi-TCR as described herein) (knockin) simultaneously removes expression of the endogenous protein (e.g., the endogenous TCR or SUV39H1) (knockout). The exogenous gene or transgene (e.g. coding for the Hi-TCR construct) can be integrated in an exonic location of the endogenous gene to be knocked out as above described. In other embodiments, the exogenous gene can be integrated in an intronic locus of the endogenous gene to be knocked out as described in WO2021/183884 (see FIGS. 9A-9C for illustration). For example, when the intronic KI strategy is close to the 5′ end of an exon. The transgene's sequence is juxtaposed to the exon and a novel splice acceptor is added. When the intronic KI strategy is close to the 3′ end of an exon, the transgene's sequence can be juxtaposed to the exon and a novel splice donor is typically added. When the intronic KI strategy in the middle of an intron, a splice acceptor and a splice donor typically add a new exon to the transcript. For these three examples the donor template constructs typically comprise a transgene flanked by a typical self-cleaving peptide (e.g., P2A, E2A, T2A, or F2A) to preserve the transcriptional regulation of the endogenous gene. A stop codon and a polyadenylation sequence can be added to the donor template construct to terminate the translation and transcription.

The desired genetic change is typically stimulated by introduction of a Cas protein (e.g., Cas9 or Cas12 protein) and a guide RNA (gRNA) ribonucleoprotein (RNP) which introduces a double-stranded or single-stranded break at the chosen gRNA sequence within the endogenous protein locus (e.g., T cell receptor alpha constant chain (TRAC) genomic locus or within the SuV39H1 locus). Repair of this break can proceed by either homology-directed-repair (HDR), which makes use of homologous DNA templates to direct repair outcomes, or by non-homologous-end-joining (NHEJ), which directly ligates the broken ends in an error-prone manner leading to frequent insertion or deletion of the surrounding bases (indels). The effect of NHEJ-mediated indels is dependent on the location of the gRNA target sequence. Those gRNAs targeting a coding sequence or nearby structural elements are prone to disrupting protein or mRNA expression, leading to NHEJ-mediated knockout of the targeted gene. The balance of NHEJ to HDR events is dependent on both the choice of gRNA target sequence and the availability of an HDR template (HDRT).

### Engineering T Cell Receptor Locus

In certain embodiments, an antigen receptor such as a HI-TCR is expressed by an immunoresponsive cell through a modified endogenous T cell receptor locus. In certain embodiments, an HI-TCR expression cassette is integrated at an endogenous T cell receptor locus. In certain embodiments, the HI-TCR expression cassette is integrated within the T cell receptor alpha locus (TRA, GenBank ID: 6955). In certain embodiments, the HI-TCR expression cassette is integrated within the T cell receptor beta locus (TRB, GenBank ID: 6957). In certain embodiments, the HI-TCR expression cassette is integrated within the T cell receptor gamma locus (TRG, GenBank ID: 6965).

In certain embodiments, the HI-TCR expression cassette comprises an extracellular antigen-binding domain that is integrated in the first exon of a TCR constant domain locus, so that the extracellular antigen-binding domain and the TCR constant domain are comprised in one antigen binding chain of the HI-TCR. In certain embodiments, the TCR constant domain locus can be TRAC, TRBC1, TRBC2, TRGC1, or TRGC2. In certain embodiments, the HI-TCR expression cassette comprises an extracellular antigen-binding domain that is integrated in the first exon of a TRAC locus, so that the extracellular antigen-binding domain and a TRAC peptide are comprised in a first antigen binding chain of the HI-TCR. In certain embodiments, the HI-TCR expression cassette further comprises a second antigen binding chain, which optionally comprises an extracellular antigen-binding domain and a TRBC peptide. In certain embodiments, the HI-TCR expression cassette comprises an extracellular antigen-binding domain that is integrated in the first exon of a TRBC locus, so that the extracellular antigen-binding domain and a TRBC peptide are comprised in a first antigen binding chain of the HI-TCR. In certain embodiments, the HI-TCR expression cassette further comprises a second antigen binding chain, which optionally comprises an extracellular antigen-binding domain and a TRAC peptide. In certain embodiments, the expression cassette comprises elements that create polycistronic expression cassette, e.g., a cleavable peptide, e.g., a 2A peptide.

In certain embodiments, the recombinant TCR is expressed from an expression cassette placed in an endogenous TRAC locus and/or a TRBC locus of an immunoresponsive cell. In certain embodiments, the placement (knockin) of the recombinant TCR expression cassette disrupts or abolishes (knock-out) the endogenous expression of a TCR comprising a native TCR alpha chain and/or a native TCR beta chain in the immunoresponsive cell. In certain embodiments, the placement of the recombinant TCR expression cassette prevents or eliminates mispairing between the recombinant TCR and a native TCR a chain and/or a native TCR b chain in the immunoresponsive cell.

In some embodiments, the transgene typically coding for the antigen receptor (e.g. a Hi-TCR) is placed at an intronic location of a TRAC and/or TRBC locus. In such embodiments, a gRNA can have a sequence having at least 85% (e.g., 85%, 87%, 89%, 91%, 93%, 95%, 97%, 99%, or 100%) identity to a sequence of any one of SEQ ID NOS:2-9 of WO2021/183884 (e.g., gRNA G526, gRNA G527, gRNA G528, gRNA G529, gRNA G530, gRNA G531, gRNA G532, and gRNA G533). In some embodiments, a gRNA can have a sequence having at least 85% (e.g., 85%, 87%, 89%, 91%, 93%, 95%, 97%, 99%, or 100%) identity to a sequence of any one of SEQ ID NOS: 17-28 of WO2021/183884 (e.g., gRNA G542, gRNA G543, gRNA G544, gRNA G545, gRNA G546, gRNA G547, gRNA G548, gRNA G549, gRNA G550, gRNA G551, gRNA G552, and gRNA G553).

### SUV Locus Engineering

The present invention also comprises the targeted integration of an expression cassette into a Suv39H1 gene site in an immune cell as herein described, preferably a human T cells or a progenitor thereof, wherein the transgene (exogenous nucleic acid) is encoding at least an engineered antigen receptor, a modified TCR (e.g. a Hi-TCR) and/or any other therapeutic protein. The transgene may be integrated in any one of exon 1 to 6 or in a non-coding regulatory region upstream of exon 1, or in any intronic region between exons 1 and 2, exons 2 and 3, exons 3 and 4, exons 4 and 5, and exons 5 and 6.

### Integration of the Exogenous Protein

Integration of the exogenous protein (e.g., an exogenous intracellular or cell surface protein (e.g., an exogenous Hi-tCR)) into a T cell at the gRNA target site can be typically directed by co-delivery of an HDRT which includes a left and right homology arm having homology to sequences flanking the genomic break (LHA and RHA, respectively) and surrounding the exogenous protein (e.g., an exogenous intracellular or cell surface protein (e.g., an exogenous Hi-TCR)) insert. In some embodiments, the exogenous protein (e.g., an exogenous intracellular or cell surface protein (e.g., an exogenous Hi-TCR)) is integrated in-frame at the endogenous cell surface protein locus (e.g., TRAC locus), following a self cleaving peptide (e.g., P2A, E2A, T2A, or F2A) typically when an intronic locus is targeted. This leads to expression of the CAR or exogenous protein (e.g., an exogenous intracellular or cell surface protein (e.g., an exogenous Hi-TCR)) insert while simultaneously interrupting expression of the endogenous cell surface protein (e.g., endogenous TCR).

Knock in efficiency is directly correlated to nuclear concentration of the HDRT and can be increased by delivering the HDRT with for example either recombinant viral vector or ssDNA/dsDNA hybrid Cas9 shuttle as defined for example in WO2021/183884. In some embodiments, the HDRT and/or the gRNA in a composition described herein can be introduced into the cell via viral delivery using a viral vector. For example, viral vectors can be based on vaccinia virus, poliovirus, adenovirus, adeno-associated virus (AAV) (e.g., recombinant AAV (rAAV)), SV40, herpes simplex virus, human immunodeficiency virus, and the like. A retroviral vector can be based on Murine Leukemia Virus, spleen necrosis virus, and vectors derived from retroviruses such as Rous Sarcoma Virus, Harvey Sarcoma Virus, avian leukosis virus, a lentivirus (e.g., integration deficient lentivirus), human immunodeficiency virus, myeloproliferative sarcoma virus, mammary tumor virus, and the like. In some embodiments, a retroviral vector can be an integration deficient gamma retroviral vector. Other useful expression vectors are known to those of skill in the art, and many are commercially available. The following exemplary vectors are provided by way of example for eukaryotic host cells: pXTI, pSG5, pSVK3, pBPV, pMSG, and pSVLSV40. Examples of techniques that may be used to introduce a viral vector into a cell include, but not limited to, viral or bacteriophage infection, transfection, protoplast fusion, lipofection, calcium phosphate precipitation, polyethyleneimine (PEI)-mediated transfection, DEAE-dextran mediated transfection, liposome-mediated transfection, calcium phosphate precipitation, nanoparticle-mediated nucleic acid delivery, and the like.

The expression cassette can be constructed with an auxiliary molecule (e.g., a cytokine) in a single, multi cistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-I IRES, FGF-2 IRES, VEGF IRES, IGF-II IRES, NF-kB IRES, RUNX1 IRES, p53 IRES, hepatitis A IRES, hepatitis C IRES, pestivirus IRES, aphthovirus IRES, picomavirus IRES, poliovirus IRES and encephalomyocarditis virus IRES) and cleavable linkers (e.g, 2 A peptides, e.g., P2A, T2A, E2A and F2A peptides).

In some embodiments, the expression cassette is “promoter-less” such that the expression of the transgene in under the control of an endogenous promoter. In some other embodiments the expression cassette includes a promoter that drives the expression of the transgene. Examples include CMV, EF-1a, hPGK and RPBSA. CAG promoters have been used to overexpress lncRNA. Yin et al Cell Stem Cell. 2015 May 7; 16(5):504-16. Inducible promoters that are driven by signals from activated T cells include nuclear factor of activated T cells (NFAT) promoter. Other promoters for T cell expression of RNA include CIFT chimeric promoter (containing portions of cytomegalovirus (CMV) enhancer, core interferon gamma (IFN-γ) promoter, JeT promoter (WO 2002/012514), and a T-lymphotropic virus long terminal repeat sequence (TLTR)), endogenous TRAC promoter or TRBC promoter. Inducible, constitutive, or tissue-specific promoters are contemplated.

In certain non-limiting embodiments, the expression of expression cassette integrated into a targeted genomic locus (e.g., SUV39H1 locus) is regulated by an endogenous transcription terminator of the genomic locus. In certain embodiments, the expression of an expression cassette integrated into a targeted genomic locus is regulated by a modified transcription terminator introduced to the genomic locus. Any targeted genome editing methods can be used to modify the transcription terminator region of a targeted genomic locus, and thereby modifying the expression of an expression cassette in an immune cell of the present invention. In certain embodiments, the modification comprises replacement of an endogenous transcription terminator with an alternative transcription terminator, or insertion of an alternative transcription terminator to the transcription terminator region of a targeted genomic locus. In certain embodiments, the alternative transcription terminator comprises a 3′UTR region or a ploy A region of a gene. In certain embodiments, the alternative transcription terminator is endogenous. In certain embodiments, the alternative transcription terminator is exogenous. In certain embodiments, alternative transcription terminators include, but are not limited to, a TK transcription terminator, a GCSF transcription terminator, a TCRA transcription terminator, an HBB transcription terminator, a bovine growth hormone transcription terminator, an SV40 transcription terminator and a P2A element.

Preferentially, the inactivation of SUV39H1 activity leads to the absence in the cell of substantial detectable activity of SUV39H1. Inactivation of SUV39H1 activity can be achieved through repression of SUV39H1 gene expression, or through mutation of the SUV39H1 gene of the cell, or through expression of, delivery of, or contact with, exogenous inhibitors. For example, repression may reduce expression of SUV39H1 in the cell by at least 50, 60, 70, 80, 90, or 95% as to the same cell produced by the method in the absence of the repression. Gene disruption (mutation) may also lead to a reduced expression of the SUV39H1 protein or to the expression of a non-functional SUV39H1 protein. Inhibition of SUV39H1 in the immune cell according to the present disclosure can be permanent and irreversible or transient or reversible. Preferably, SUV39H1 inhibition is permanent and irreversible. Inhibition of SUV39H1 in the cell may be achieved prior or after injection of the cell in the targeted patient as described below.

In some embodiments, the inhibition of SUV39H1 activity in the engineered immune cell disclosed herein is achieved by delivering or expressing at least one agent that inhibits or blocks the expression and/or activity of SUV39H1, i.e. a “SUV39H1 inhibitor.”

Small molecule SUV39H1 inhibitors are known, for example, inhibitors of epipolythiodioxopiperazine (ETP) class of methyltransferase inhibitors, such as chaetocin, ETP69, or other epidithiodioxopiperazine alkaloids.

One inhibitor of H3K9-histone methyltransferase SUV39H1 is chaetocin (CAS 28097-03-2) as described by Greiner D, Bonaldi T, Eskeland R, Roemer E, Imhof A. “Identification of a specific inhibitor of the histone methyltransferase SU(VAR)3-9”. Nat Chem Biol. 2005 August; I(3): 143-5.; Weber, H. P., et al, “The molecular structure and absolute configuration of chaetocin”, Acta Cryst, B28, 2945-2951 (1972); Udagawa, S., et al, “The production of chaetoglobosins, sterigmatocystin, O-methylsterigmatocystin, and chaetocin by Chaetomium spp. and related fungi”, Can. J. microbiol, 25, 170-177 (1979); and Gardiner, D. M., et al, “The epipolythiodioxopiperazine (ETP) class of fungal toxins: distribution, mode of action, functions and biosynthesis”, Microbiol, 151, 1021-1032 (2005). Chaetocin is commercially available from Sigma Aldrich.

An inhibitor of Suv39h1 can also be ETP69 (Rac-(3S,6S,7S,8aS)-6-(benzo[d][1,3]dioxol-5-yl)-2,3,7-trimethyl-1,4-dioxohexahydro-6H-3,8a-epidithiopyrrolo[1,2-a]pyrazine-7-carbonitrile), a racemic analog of the epidithiodiketopiperazine alkaloid chaetocin A (see WO2014066435 but see also Baumann M, Dieskau A P, Loertscher B M, et al. Tricyclic Analogues of Epidithiodioxopiperazine Alkaloids with Promising In Vitro and In Vivo Antitumor Activity. Chemical science (Royal Society of Chemistry: 2010). 2015; 6:4451-4457, and Snigdha S, Prieto G A, Petrosyan A, et al. H3K9me3 Inhibition Improves Memory, Promotes Spine Formation, and Increases BDNF Levels in the Aged Hippocampus. The Journal of Neuroscience. 2016; 36(12):3611-3622).

The inhibiting activity of a compound may be determined using various methods as described in Greiner D. Et al. Nat Chem Biol. 2005 August; I(3): 143-5 or Eskeland, R. et al. Biochemistry 43, 3740-3749 (2004).

Inhibition of Suv39h1 in the cell can be achieved before or after injection in the patient or subject. In some embodiment, inhibition as previously defined is performed in vivo after administration of the cell to the subject. Alternatively, a Suv39h1 inhibitor as herein defined can be included in the composition containing the cell. Suv39h1 may also be administered separately before, concomitantly of after administration of the cell(s) to the subject.

In some embodiments, inhibition of Suv39h1 according to the invention may be achieved with incubation of a cell according to the invention with a composition containing at least one pharmacological inhibitor as previously described. The inhibitor is included during the expansion of the anti-tumor T cells in vitro, thus modifying their reconstitution, survival and therapeutic efficacy after adoptive transfer.

Other suitable SUV39H1 inhibitors include, for example, agents that hybridize or bind to the SUV39H1 gene or its regulatory elements, such as aptamers that block or inhibit SUV39H1 expression or activity; nucleic acid molecules that block transcription or translation, such as antisense molecules complementary to SUV39H1; RNA interfering agents (such as a small interfering RNA (siRNA), small hairpin RNA (shRNA), Long noncoding RNAs (lncRNAs), microRNA (miRNA), or a piwiRNA (piRNA); ribozymes and combination thereof. It has been shown that antisense lncRNA, AF196970.3 has silencing function against SUV39H1 in human cells, inhibiting SUV39H1 expression and thus lowering levels of SUV39H1 protein in the cell. AF196970.3 has an expression pattern very similar to the expression pattern of SUV39H1. It is detected in many different cell types, with endothelial cells, fibroblasts and myocytes showing the highest levels, similar to SUV39H1. The gene locus of SUV39H1 is on the X chromosome (position p11.23, 48695554-48709016, in the GRCh38.p13 assembly). At the same locus, in anti-parallel orientation, the non-annotated gene ENSG00000232828 is located at positions 48698963-48737163. Both genes have annotated promoter regions. SEQ ID NO: 55: lncRNA AF196970.3 RNA sequence: (CGAGGCAGGGCUUUGGCUACUGGAGAUCGUAGGUUCGAAUCCCGUCUGGGAA GUUCAACUUGUGCACCUGUAAAAGAAGCUGGCAUUAUUGGCUUGUACUCAAGG GCUGGCACAGAGUGUGUCGGGGUGCGGACGCCCCAGCCACGCCCAUCAUCCGU GCGGAAGAUGCAGAGGUCAUAUCGGAUACCCUUCUGUACCACACGAUUUGGGC AGUCAUAGCCGCAGCGGCAGCGGGAGUUGCACUCGUAGAUGGGCAGCCCGGCU CGAAGCCGCACCUGGCCCUGGUCAUUGUAGGCAAACUUGUGCAGUGACGCCCC CGGGCAGCAGCCUCCAGUGGGUGCCCACAGACAGUCCUGGCACUCGCAGCCCA CAGCCACCUGGUUGAGGGUGAUGCCCUCACCAACACGGUACUCAUUGAUGUAC ACGAAGGCCCGCGGAGGGCCGUCCAGGUCCACCUCAUUCUCUACAGUGAUGCG UCCCAGAUGGCUGCGCUUGGCAUUGAGCUCCUGCUCCCAGCGACGGAGCGCCC GCCUCUGCUUGGCCUUCUGCACCAGGUAGUUGGCCAAGCUUGGGUCCAGGUGC CGGGGGGUCUUUGACCGGUGGUGCCGCCGGAGCAGCUCCCUUUCUAAGUCCU UGUGGAACUGCUUGAGGAUACGCACACACUUGAGAUUCUGCCGUGGCUCCCAG GUGCUCUCUGAGUCUGGAUAUCCACGCCAUUUCACCAGGGUCAAAGGAGAAAAU UCCCUUUGGAAACAGAUGUGGGCAGUUGGGGACAAGAGGGCAGGACACUAACU UCCUUGUGACCUGUCCCCUCCCAGAGCAUGGUCACCCCAGACUCACGCGGAUC UUCUUGUAAUCGCACAGGUACUCGACUUCAAAGUCAUAGAGGUUCCUCUUAGAG AUACCGAGGGCAGGGCAGGAGAGC) is the predicted RNA sequence expressed from ENSG00000232828, after transcription and splicing. SEQ ID NO: 1 is a 925 base sequence that includes three exons. AF196970.3 exon 1 is 125 bases in length and does not have significant complementarity to the SUV39H1 gene. AF196970.3 exon 2 is 600 bases in length and is anti-parallel to a substantial portion of exon 3 of the SUV39HI gene with 100% similarity. AF196970.3 exon 3 is 200 bases in length and is anti-parallel to a portion (42.5% similarity) of exon 2 of the SUV39G1 gene, as well as a portion of the adjacent intron. Inhibitory polynucleotides of the disclosure, for use in the cells and methods of the disclosure, include AF196970.3 (SEQ ID NO: 1) or fragments or variants thereof.

Suitable SUV39H1 inhibitors can also include an exogenous nucleic acid comprising a) an engineered, non-naturally occurring Clustered Regularly Interspaced Short Palindromic Repeats (CRISPR) guide RNA that hybridizes with SUV39H1 genomic nucleic acid sequence and/or b) a nucleotide sequence encoding a CRISPR protein (typically a Type-II Cas9 protein), optionally wherein the cells are transgenic for expressing a Cas9 protein, or an RNP comprising the guide RNA and the CRISPR protein. The agent may also be a Zinc finger protein (ZF) or a TAL protein. The Cas9 protein, TAL protein and/or ZF protein are linked directly or indirectly to a repressor and/or inhibitor, or are linked to a nuclease that confers gene editing activity

Suitable SUV39H1 inhibitors can also include non-functional SUV39H1. In some embodiments, the wildtype SUV39H1 gene is not inactivated, but rather a SUV39H1 inhibitor is expressed in the cell. In some embodiments the inhibitor is a dominant negative SUV39H1 gene that expresses non-functional gene product at a level that inhibits activity of the wildtype SUV39H1. This may comprise overexpression of the dominant negative SUV39H1.

The inactivation of SUV39H1 in the immune cell and the introduction of an antigen-specific receptor that specifically binds to a target antigen can be carried out simultaneously or sequentially in any order.

Inactivation of SUV39H1 in a cell according to the disclosure may also be effected via repression or disruption of the SUV39H1 gene, such as by deletion, e.g., deletion of the entire gene, exon, or region, and/or replacement with an exogenous sequence, and/or by mutation, e.g., frameshift or missense mutation, within the gene, typically within an exon of the gene. In some embodiments, the disruption results in a premature stop codon being incorporated into the gene, such that the SUV39H1 protein is not expressed or is non-functional. The disruption is generally carried out at the DNA level. The disruption generally is permanent, irreversible, or not transient.

In some embodiments, the gene inactivation is achieved using gene editing agents such as a DNA-targeting molecule, such as a DNA-binding protein or DNA-binding nucleic acid, or complex, compound, or composition, containing the same, which specifically binds to or hybridizes to the gene. In some embodiments, the DNA-targeting molecule comprises a DNA-binding domain, e.g., a zinc finger protein (ZFP) DNA-binding domain, a transcription activator-like protein (TAL) or TAL effector (TALE) DNA-binding domain, a clustered regularly interspaced short palindromic repeats (CRISPR) DNA-binding domain, or a DNA-binding domain from a meganuclease.

Zinc finger, TALE, and CRISPR system binding domains can be “engineered” to bind to a predetermined nucleotide sequence.

In some embodiments, the DNA-targeting molecule, complex, or combination contains a DNA-binding molecule and one or more additional domain, such as an effector domain to facilitate the repression or disruption of the gene. For example, in some embodiments, the gene disruption is carried out by fusion proteins that comprise DNA-binding proteins and a heterologous regulatory domain or functional fragment thereof.

Typically, the additional domain is a nuclease domain. Thus, in some embodiments, gene disruption is facilitated by gene or genome editing, using engineered proteins, such as nucleases and nuclease-containing complexes or fusion proteins, composed of sequence-specific DNA-binding domains fused to, or complexed with, non-specific DNA-cleavage molecules such as nucleases.

These targeted chimeric nucleases or nuclease-containing complexes carry out precise genetic modifications by inducing targeted double-stranded breaks or single-stranded breaks, stimulating the cellular DNA-repair mechanisms, including error-prone nonhomologous end joining (NHEJ) and homology-directed repair (HDR). In some embodiments the nuclease is an endonuclease, such as a zinc finger nuclease (ZFN), TALE nuclease (TALEN), an RNA-guided endonuclease (RGEN), such as a CRISPR-associated (Cas) protein, or a meganuclease. Such systems are well-known in the art (see, for example, U.S. Pat. No. 8,697,359; Sander and Joung (2014) Nat. Biotech. 32:347-355; Hale et al. (2009) Cell 139:945-956; Karginov and Hannon (2010) Mol. Cell 37:7; U.S. Pat. Publ. 2014/0087426 and 2012/0178169; Boch et al. (2011) Nat. Biotech. 29:135-136; Boch et al. (2009) Science 326:1509-1512; Moscou and Bogdanove (2009) Science 326: 1501; Weber et al. (2011) PLoS One 6:e19722; Li et al. (2011) Nucl. Acids Res. 39:6315-6325; Zhang et al. (2011) Nat. Biotech. 29: 149-153; Miller et al. (2011) Nat. Biotech. 29: 143-148; Lin et al. (2014) Nucl. Acids Res. 42:e47). Such genetic strategies can use constitutive expression systems or inducible expression systems according to well-known methods in the art.

### ZFPs and ZFNs; TALs, TALEs, and TALENs

In some embodiments, the DNA-targeting molecule includes a DNA-binding protein such as one or more zinc finger protein (ZFP) or transcription activator-like protein (TAL), fused to an effector protein such as an endonuclease. Examples include ZFNs, TALEs, and TALENs. See Lloyd et al., Frontiers in Immunology, 4(221), 1-7 (2013).

In some embodiments, the DNA-targeting molecule comprises one or more zinc-finger proteins (ZFPs) or domains thereof that bind to DNA in a sequence-specific manner. A ZFP or domain thereof is a protein or domain within a larger protein, that binds DNA in a sequence-specific manner through one or more zinc fingers regions of amino acid sequence within the binding domain whose structure is stabilized through coordination of a zinc ion. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to the target site of choice. See, for example, Beerli et al. (2002) Nature Biotechnol. 20: 135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416.

In some embodiments, the DNA-targeting molecule is or comprises a zinc-finger DNA binding domain fused to a DNA cleavage domain to form a zinc-finger nuclease (ZFN). In some embodiments, fusion proteins comprise the cleavage domain (or cleavage half-domain) from at least one Type IIS restriction enzyme and one or more zinc finger binding domains, which may or may not be engineered. In some embodiments, the cleavage domain is from the Type IIS restriction endonuclease Fok I. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982.

In some aspects, the ZFNs efficiently generate a double strand break (DSB), for example at a predetermined site in the coding region of the targeted gene (i.e. SUV39H1). Typical targeted gene regions include exons, regions encoding N-terminal regions, first exon, second exon, and promoter or enhancer regions. In some embodiments, transient expression of the ZFNs promotes highly efficient and permanent disruption of the target gene in the engineered cells. In particular, in some embodiments, delivery of the ZFNs results in the permanent disruption of the gene with efficiencies surpassing 50%. Many gene-specific engineered zinc fingers are available commercially. For example, Sangamo Biosciences (Richmond, CA, USA) has developed a platform (CompoZr) for zinc-finger construction in partnership with Sigma-Aldrich (St. Louis, MO, USA), allowing investigators to bypass zinc-finger construction and validation altogether, and provides specifically targeted zinc fingers for thousands of proteins. Gaj et al., Trends in Biotechnology, 2013, 31(7), 397-405. In some embodiments, commercially available zinc fingers are used or are custom designed. (See, for example, Sigma-Aldrich catalog numbers CSTZFND, CSTZFN, CT11-1 KT, and PZD0020).

In some embodiments, the DNA-targeting molecule comprises a naturally occurring or engineered (non-naturally occurring) transcription activator-like protein (TAL) DNA binding domain, such as in a transcription activator-like protein effector (TALE) protein, See, e.g., U.S. Patent Publication No. 20110301073. In some embodiments, the molecule is a DNA binding endonuclease, such as a TALE-nuclease (TALEN). In some aspects the TALEN is a fusion protein comprising a DNA-binding domain derived from a TALE and a nuclease catalytic domain to cleave a nucleic acid target sequence. In some embodiments, the TALE DNA-binding domain has been engineered to bind a target sequence within genes that encode the target antigen and/or the immunosuppressive molecule. For example, in some aspects, the TALE DNA-binding domain may target CD38 and/or an adenosine receptor, such as A2AR.

In some embodiments, the TALEN recognizes and cleaves the target sequence in the gene. In some aspects, cleavage of the DNA results in double-stranded breaks. In some aspects the breaks stimulate the rate of homologous recombination or non-homologous end joining (NHEJ). Generally, NHEJ is an imperfect repair process that often results in changes to the DNA sequence at the site of the cleavage. In some aspects, repair mechanisms involve rejoining of what remains of the two DNA ends through direct re-ligation (Critchlow and Jackson, Trends Biochem Sci. 1998 October; 23(10):394-8) or via the so-called microhomology-mediated end joining. In some embodiments, repair via NHEJ results in small insertions or deletions and can be used to disrupt and thereby repress the gene. In some embodiments, the modification may be a substitution, deletion, or addition of at least one nucleotide. In some aspects, cells in which a cleavage-induced mutagenesis event, i.e. a mutagenesis event consecutive to an NHEJ event, has occurred can be identified and/or selected by well-known methods in the art.

TALE repeats can be assembled to specifically target the SUV39H1 gene. (Gaj et al., Trends in Biotechnology, 2013, 31(7), 397-405). A library of TALENs targeting 18,740 human protein-coding genes has been constructed (Kim et al., Nature Biotechnology. 31, 251-258 (2013)). Custom-designed TALE arrays are commercially available through Cellectis Bioresearch (Paris, France), Transposagen Biopharmaceuticals (Lexington, KY, USA), and Life Technologies (Grand Island, NY, USA). Specifically, TALENs that target CD38 are commercially available (See Gencopoeia, catalog numbers HTN222870-1, HTN222870-2, and HTN222870-3, available on the World Wide Web at www.genecopoeia.com/product/search/detail.php?prt=26&cid=&key=HTN222870). Exemplary molecules are described, e.g., in U.S. Patent Publication Nos. US 2014/0120622, and 2013/0315884.

In some embodiments the TALENs are introduced as transgenes encoded by one or more plasmid vectors. In some aspects, the plasmid vector can contain a selection marker which provides for identification and/or selection of cells which received said vector.

### RGENs (CRISPR/Cas Systems)

The gene repression can be carried out using one or more DNA-binding nucleic acids, such as disruption via an RNA-guided endonuclease (RGEN), or other form of repression by another RNA-guided effector molecule. For example, in some embodiments, the gene repression can be carried out using clustered regularly interspaced short palindromic repeats (CRISPR) and CRISPR-associated proteins. See Sander and Joung, Nature Biotechnology, 32(4): 347-355.

In general, “CRISPR system” refers collectively to transcripts and other elements involved in the expression of, or directing the activity of, CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

Typically, the CRISPR/Cas nuclease or CRISPR/Cas nuclease system includes a non-coding RNA molecule (guide) RNA, which sequence-specifically binds to DNA, and a CRISPR protein, with nuclease functionality (e.g., two nuclease domains). One or more elements of a CRISPR system can derive from a type I, type II, or type III CRISPR system, such as Cas nuclease. Preferably, the CRISPR protein is a Cas enzyme such as Cas9. Cas enzymes are well-known in the field; for example, the amino acid sequence of S. pyogenes Cas9 protein may be found in the SwissProt database under accession number Q99ZW2. In some embodiments, a Cas nuclease and gRNA are introduced into the cell. In some embodiments, the CRISPR system induces DSBs at the target site, followed by disruptions as discussed herein. In other embodiments, Cas9 variants, deemed “nickases” can be used to nick a single strand at the target site. Paired nickases can also be used, e.g., to improve specificity, each directed by a pair of different gRNAs targeting sequences. In still other embodiments, catalytically inactive Cas9 can be fused to a heterologous effector domain, such as a transcriptional repressor, to affect gene expression.

In general, a CRISPR system is characterized by elements that promote the formation of a CRISPR complex at the site of the target sequence. Typically, in the context of formation of a CRISPR complex, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex. The target sequence may comprise any polynucleotide, such as DNA or RNA polynucleotides. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

In some embodiments, one or more vectors driving expression of one or more elements of the CRISPR system are introduced into the cell such that expression of the elements of the CRISPR system direct formation of the CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. In some embodiments, CRISPR system elements that are combined in a single vector may be arranged in any suitable orientation. In some embodiments, the CRISPR enzyme, guide sequence, tracr-mate sequence, and tracr sequence are operably linked to and expressed from the same promoter. In some embodiments, a vector comprises a regulatory element operably linked to an enzyme-coding sequence encoding the CRISPR enzyme, such as a Cas protein.

In some embodiments, a CRISPR enzyme in combination with (and optionally complexed with) a guide sequence is delivered to the cell. Typically, CRISPR/Cas9 technology may be used to knockdown gene expression of SUV39H1 in the engineered cells. For example, Cas9 nuclease and a guide RNA specific to the SUV39H1 gene can be introduced into cells, for example, using lentiviral delivery vectors or any of a number of known delivery method or vehicle for transfer to cells, such as any of a number of known methods or vehicles for delivering Cas9 molecules and guide RNAs (see also below).

### Delivery of Nucleic Acids Encoding the Gene Disrupting Molecules and Complexes

In some embodiments, a nucleic acid encoding the DNA-targeting molecule, complex, or combination, is administered or introduced to the cell. Typically, viral and non-viral based gene transfer methods can be used to introduce nucleic acids encoding components of a CRISPR, ZFP, ZFN, TALE, and/or TALEN system to cells in culture.

In some embodiments, the polypeptides are synthesized in situ in the cell as a result of the introduction of polynucleotides encoding the polypeptides into the cell. In some aspects, the polypeptides could be produced outside the cell and then introduced thereto.

Methods for introducing a polynucleotide construct into animal cells are known and include, as non-limiting examples, stable transformation methods wherein the polynucleotide construct is integrated into the genome of the cell, transient transformation methods wherein the polynucleotide construct is not integrated into the genome of the cell, and virus mediated methods.

In some embodiments, the polynucleotides may be introduced into the cell by for example, recombinant viral vectors (e.g. retroviruses, adenoviruses), liposome and the like. Transient transformation methods include microinjection, electroporation, or particle bombardment. The nucleic acid is administered in the form of an expression vector. Preferably, the expression vector is a retroviral expression vector, an adenoviral expression vector, a DNA plasmid expression vector, or an AAV expression vector.

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

RNA or DNA viral-based systems include retroviral, lentivirus, adenoviral, adeno-associated and herpes simplex virus vectors for gene transfer.

For a review of gene therapy procedures, see Anderson, Science 256:808-813 (1992); Nabel & Feigner, TIBTECH 11:211-217 (1993); Mitani & Caskey, TIBTECH 11: 162-166 (1993); Dillon. TIBTECH 11: 167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds) (1995); and Yu et al., Gene Therapy 1: 13-26 (1994).

A reporter gene which includes but is not limited to glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into the cell to encode a gene product which serves as a marker by which to measure the alteration or modification of expression of the gene product.

Genetic modification of an immunoresponsive cell as herein defined (e.g., a T cell or an NKT cell) can be accomplished by transducing a substantially homogeneous cell composition with a recombinant DNA construct. In some embodiments, a retroviral vector (either gamma-retroviral or lentiviral) can be employed for the introduction of the DNA construct into the cell. For example, a polynucleotide encoding an antigen receptor such as a Hi-TCR as herein disclosed can be cloned into a retroviral vector and expression can be driven from its endogenous promoter, from the retroviral long terminal repeat, or from a promoter specific for a target cell type of interest. Non-viral vectors may be used as well.

For initial genetic modification of an immunoresponsive cell to include an HI-TCR, a retroviral vector is generally employed for transduction, however any other suitable viral vector or non-viral delivery system can be used. The HI-TCR can be constructed with an auxiliary molecule (e.g., a cytokine) in a single, multi cistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-I IRES, FGF-2 IRES, VEGF IRES, IGF-II IRES, NF-kB IRES, RUNX1 IRES, p53 IRES, hepatitis A IRES, hepatitis C IRES, pestivirus IRES, aphthovirus IRES, picomavirus IRES, poliovirus IRES and encephalomyocarditis virus IRES) and cleavable linkers (e.g, 2 A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller, et /. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464).

In some embodiments, AAV constructs can be used for targeted delivery of a antigen receptor construct (e.g. a Hi-TCR construct) as herein described in a specific locus. For example, well suited sequences according to the present application are described in Mansilla-Soto, J., Eyquem, J., Haubner, S. et aL. HLA-independent T cell receptors for targeting tumors with low antigen density. Nat Med 28, 345-352 (2022).

Non-amphotropic particles are suitable too, e.g., particles pseudotyped with VSVG, RD114 or GALV envelope and any other known in the art.

Possible methods of transduction also include direct co-culture of the cells with producer cells, e.g., by the method ofBregni, et al. (1992) Blood 80: 1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89: 1817.

Other transducing viral vectors can be used to modify an immunoresponsive cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S. A. 94: 10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244: 1275-1281, 1989; Eglitis et al., BioTechniques 6:608-614, 1988; Tolstoshev et al., Current Opinion in Biotechnology 1:55-61, 1990; Sharp, The Lancet 337: 1277-1278, 1991; Cometta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of an immunoresponsive cell. For example, a nucleic acid molecule can be introduced into an immunoresponsive cell by administering the nucleic acid in the presence of lipofection (Feigner et al., Proc. Natl. Acad. Sci. U.S. A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263: 14621, 1988; Wu et al., Journal of Biological Chemistry 264: 16985, 1989), or by micro-injection under surgical conditions (Wolff et al., Science 247: 1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically.

Recombinant receptors can also be derived or obtained using transposases or targeted nucleases (e.g. Zinc finger nucleases, meganucleases, or TALE nucleases, CRISPR). Transient expression may be obtained by RNA electroporation. In certain embodiments, recombinant receptors can be introduced by a transposon-based vector. In certain embodiments, the transposon-based vector comprises a transposon (a.k.a. a transposable element). In certain embodiments, the transposon can be recognized by a transposase. In certain embodiments, the transposase is a Sleeping Beauty transposase.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type Ils restriction endonuclease Fokl. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor Ia enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

### Cell Preparation

Isolation of the cells includes one or more preparation and/or non-affinity based cell separation steps according to well-known techniques in the field. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some embodiments, the cell preparation includes steps for freezing, e.g., cryopreserving, the cells, either before or after isolation, incubation, and/or engineering. Any of a variety of known freezing solutions and parameters in some aspects may be used.

The incubation steps can comprise culture, incubation, stimulation, activation, expansion and/or propagation.

In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a antigen-specific receptor.

The incubation conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include 1L-2 and/or IL-15, for example, an IL-2 concentration of at least about 10 units/mL.

In some aspects, incubation is carried out in accordance with techniques such as those described in U.S. Pat. No. 6,040,177 to Riddell et al., Klebanoff et al., J Immunother. 2012; 35(9): 651-660, Terakura et al., Blood. 2012; 1:72-82, and/or Wang et al. J Immunother. 2012, 35(9):689-701.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

In some aspects, the methods include assessing expression of one or more markers on the surface of the engineered cells or cells being engineered. In one embodiment, the methods include assessing surface expression of one or more target antigen (e.g., antigen recognized by the antigen-specific receptor) sought to be targeted by the adoptive cell therapy, for example, by affinity-based detection methods such as by flow cytometry.

### Vectors and Methods for Cell Genetic Engineering

In some aspects, the genetic engineering involves introduction of a nucleic acid encoding the genetically engineered component or other component for introduction into the cell, such as a component encoding a gene-disruption protein or nucleic acid.

Generally, the engineering of CARs into immune cells (e.g., T cells) requires that the cells be cultured to allow for transduction and expansion. The transduction may utilize a variety of methods, but stable gene transfer is required to enable sustained CAR expression in clonally expanding and persisting engineered cells.

In some embodiments, gene transfer is accomplished by first stimulating cell growth, e.g., T cell growth, proliferation, and/or activation, followed by transduction of the activated cells, and expansion in culture to numbers sufficient for clinical applications.

Various methods for the introduction of genetically engineered components, e.g., antigen-specific receptors, e.g., CARs, are well known and may be used with the provided methods and compositions. Exemplary methods include those for transfer of nucleic acids encoding the receptors, including via viral, e.g., retroviral or lentiviral, transduction, transposons, and electroporation.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3.; Carlens et al. (2000) Exp Hematol 28(10): 1137-46; Alonso-Camino et al. (2013) Mol Ther Nucl Acids 2, e93; Park et al., Trends Biotechnol. 2011 November; 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3: 102-109.

Methods of lentiviral transduction are also known. Exemplary methods are described in, e.g., Wang et al. (2012) J. Immunother. 35(9): 689-701; Cooper et al. (2003) Blood. 101: 1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation {see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the genetically engineered nucleic acids encoding the genetically engineered products are those described, e.g., in international patent application, Publication No.: WO2014055668, and U.S. Pat. No. 7,446,190. The presently disclosed subject matter further provides methods for producing an antigen-specific immunoresponsive cell. In certain embodiments, the method comprises introducing into an immunoresponsive cell a nucleic acid sequence encoding a recombinant TCR described herein. The nucleic acid sequence can be comprised in a vector. In certain embodiments, the expression cassette of at least one antigen binding chain of the recombinant TCR is placed at an endogenous gene locus of the immunoresponsive cell. In certain embodiments, the expression cassettes of two antigen binding chains of the recombinant TCR are placed at an endogenous gene locus of the immunoresponsive cell, wherein the two antigen binding chains are capable of dimerization. The endogenous gene locus can be a CD3S locus, a CD3s locus, a CD247locus, a B2M locus, a TRAC locus, a TRBC locus, a TRDC locus and/or a TRGC locus. In certain embodiments, the endogenous gene locus is a TRAC locus or a TRBC locus. In certain embodiments, the placement of the expression cassette of the recombinant TCR disrupts or abolishes the endogenous expression of a TCR comprising a native TCR a chain and/or a native TCR b chain in the immunoresponsive cell, whereby preventing or eliminating mispairing between the recombinant TCR and a native TCR a chain and/or a native TCR b chain in the immunoresponsive cell. In certain embodiments, the endogenous gene locus comprises a modified transcription terminator region. In certain embodiments, the modified transcription terminator region comprises a genomic element selected from the group consisting of a TK transcription terminator, a GCSF transcription terminator, a TCRA transcription terminator, an HBB transcription terminator, a bovine growth hormone transcription terminator, an SV40 transcription terminator and a P2A element. In certain embodiments, when one endogenous T cell receptor locus in a cell is modified to express the at least one antigen binding chain of the recombinant TCR, one or more other endogenous T cell receptor locus in the cell is modified to eliminate the expression of an endogenous TCR chain.

In certain embodiments, the one or more other endogenous T cell receptor locus are further modified to express a gene of interest. In certain embodiments, the gene of interest is an anti-tumor cytokine, a co-stimulatory molecule ligand, a tracking gene or a suicide gene. The presently disclosed subject matter further provides nucleotide acids encoding a recombinant TCR described herein, and nucleic acid compositions comprising a recombinant TCR described herein. In certain embodiments, the nucleic acid sequences are comprised in a vector.

The presently disclosed subject matter also provides vectors comprising the nucleic acid composition described herein. Further provided are kits comprising a recombinant TCR described herein, an immunoresponsive cell described herein, a pharmaceutical composition described herein, a nucleic acid composition described herein, or a vector described herein. In certain embodiments, the kit further comprises written instructions for treating and/or preventing a neoplasm, a pathogen infection, an autoimmune disorder, or an allogeneic transplant.

### Compositions

The present disclosure also includes compositions containing the cells as described herein and/or produced by the provided methods. Typically, said compositions are pharmaceutical compositions and formulations for administration, preferably sterile compositions and formulations, such as for adoptive cell therapy.

A pharmaceutical composition of the disclosure generally comprises at least one engineered immune cell of the disclosure and a sterile pharmaceutically acceptable carrier.

As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can further be incorporated into the compositions. In some aspects, the choice of carrier in the pharmaceutical composition is determined in part by the particular engineered CAR or TCR, vector, or cells expressing the CAR or TCR, as well as by the particular method used to administer the vector or host cells expressing the CAR. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001 to about 2% by weight of the total composition.

A pharmaceutical composition is formulated to be compatible with its intended route of administration.

### Therapeutic Methods

The present disclosure also relates to the cells as previously defined for their use in adoptive cell therapy (notably adoptive T cell therapy), typically in the treatment of cancer in a subject in need thereof, but also in the treatment of infectious diseases and autoimmune, inflammatory or allergic diseases. Treatment of any of the diseases listed above under the “Antigen” section is contemplated.

The immune cells, particularly T-cells or NK cells, in which SUV39H1 has been inactivated, exhibit an enhanced central memory phenotype, enhanced survival and persistence after adoptive transfer, and reduced exhaustion. Their increased efficiency and efficacy may allow them to be dosed at lower levels relative to such cells that do not have the improvements described herein. Thus, T-cells or NK cells in which SUV39H1 has been inactivated, which optionally have any of the other features described herein (e.g. expressing a CAR, and/or in which a T cell receptor (TCR) alpha constant region gene is inactivated by the insertion of a nucleic acid sequence encoding a CAR or TCR, and/or in which the CAR comprises a) an extracellular antigen-binding domain, b) a transmembrane domain, c) optionally one or more costimulatory domains, and d) an intracellular signaling domain comprising a modified CD3zeta intracellular signaling domain in which ITAM2 and ITAM3 have been inactivated or deleted and/or in which an HLA-A gene has been inactivated or deleted), may be administered at certain doses. For example, the immune cells (e.g., T cells) in which SUV39h1 has been inactivated may be administered to adults at doses of less than about 108 cells, less than about 5×107 cells, less than about 107 cells, less than about 5×106 cells, less than about 106 cells, less than about 5×105 cells or less than about 105 cells. The dose for pediatric patients may be about 100-fold less. In alternative embodiments, any of the immune cells (e.g. T-cells or NK cells) described herein may be administered to patients at doses ranging from 105 to 109 cells, or 105 to 108 cells, or 106 to 108 cells.

The subject of the disclosure (i.e. patient) is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent. In some examples, the patient or subject is a validated animal model for disease, adoptive cell therapy, and/or for assessing toxic outcomes such as cytokine release syndrome (CRS). In some embodiments of the disclosure, said subject has a cancer, is at risk of having a cancer, or is in remission of a cancer.

The cells of the disclosure are particularly beneficial when administered to treat a chronic disease, such as a chronic infectious disease, or when administered to treat refractory, relapsed or resistant cancer.

In some embodiments, the patient exhibits a cancer relapse or is likely to exhibit a cancer relapse. In some embodiments, the patient exhibits cancer metastasis or is likely to exhibit cancer metastasis. In some embodiments, the patient has not achieved sustained cancer remission after one or more prior cancer therapies. In some embodiments, the patient suffers from a cancer that is resistant or nonresponsive to one or more prior cancer therapies. In some embodiments, the patient suffers from a refractory cancer. In some embodiments, the patient is likely to exhibit a response to cell therapy that is not durable. In some embodiments, the patient is ineligible for immune checkpoint therapy or did not respond to immune checkpoint therapy. In some embodiments, the patient is ineligible for treatment with high dose of chemotherapy and/or is ineligible for treatment with high adoptive cell therapy doses.

In certain embodiments, immune cells comprising a modified TCR disclosed herein can be used to treat a subject having tumor cells with a low expression level of a surface antigen. In some condition, the antigen is expressed at a low density because of a a relapse of a disease, typically wherein the subject received treatment which leads to residual tumor cells. In certain embodiments, the tumor cells have low density of a target molecule on the surface of the tumor cells because the targeted antigen, typically the tumor antigen is expressed at low density (such as e.g. e-antigens as previously defined). In certain embodiments, a target molecule having a low density on the cell surface has a density of less than about 5,000 molecules per cell, less than about 4,000 molecules per cell, less than about 3,000 molecules per cell, less than about 2,000 molecules per cell, less than about 1,500 molecules per cell, less than about 1,000 molecules per cell, less than about 500 molecules per cell, less than about 200 molecules per cell, or less than about 100 molecules per cell. In certain embodiments, a target molecule having a low density on the cell surface has a density of less than about 2,000 molecules per cell. In certain embodiments, a target molecule having a low density on the cell surface has a density of less than about 1,500 molecules per cell. In certain embodiments, a target molecule having a low density on the cell surface has a density of less than about 1,000 molecules per cell. In certain embodiments, a target molecule having a low density on the cell surface has a density of between about 4,000 molecules per cell and about 2,000 molecules per cell, between about 2,000 molecules per cell and about 1,000 molecules per cell, between about 1,500 molecules per cell and about 1,000 molecules per cell, between about 2,000 molecules per cell and about 500 molecules per cell, between about 1,000 molecules per cell and about 200 molecules per cell, or between about 1,000 molecules per cell and about 100 molecules per cell.

In certain embodiments, immunoresponsive cells comprising a HI-TCR disclosed herein can be used to treat a subject having a relapse of a disease. Typically, the cell also comprises a recombinant booster sequence (CCR) as previously defined herein. Typically, such cell is deficient for Suv39, notably for suv39h1. In certain embodiments, the tumor cells have a low density of a tumor specific antigen (such as CD19, CD22, CD70 or any e antigen as previously defined) on the surface of the tumor cells.

In some embodiments, an immune cell expressing an antigen receptor (e.g a Hi T cell antigen receptor) as herein described can be used for the treatment of a patient having a median value of less than less than about 6,000 molecules of the target antigen per cell. In some embodiments, the antigen is expressed at a density (typically median value) of less than about 5,000 molecules, less than about 4,000 molecules, less than about 3,000 molecules, less than about 2,000 molecules, less than about 1,000 molecules, or less than about 500 molecules of the target antigen per cell. In some embodiments, the antigen is expressed at a density (typically median value) of less than about 2,000 molecules, such as e.g., less than about 1,800 molecules, less than about 1,600 molecules, less than about 1,400 molecules, less than about 1,200 molecules, less than about 1,000 molecules, less than about 800 molecules, less than about 600 molecules, less than about 400 molecules, less than about 200 molecules, or less than about 100 molecules of the target antigen per cell. In some embodiments, the antigen is expressed at a density of less than about 1,000 molecules, such as e.g., less than about 900 molecules, less than about 800 molecules, less than about 700 molecules, less than about 600 molecules, less than about 500 molecules, less than about 400 molecules, less than about 300 molecules, less than about 200 molecules, or less than about 100 molecules of the target antigen per cell. In some embodiments, the antigen is expressed at a density ranging from about 5,000 to about 100 molecules of the target antigen per cell, such as e.g., from about 5,000 to about 1,000 molecules, from about 4,000 to about 2,000 molecules, from about 3,000 to about 2,000 molecules, from about 4,000 to about 3,000 molecules, from about 3,000 to about 1,000 molecules, from about 2,000 to about 1,000 molecules, from about 1,000 to about 500 molecules, from about 500 to about 100 molecules of the target antigen per cell. Quantification of the target antigen density per cell can be achieved as described in Jasper, G. A., Arun, I., Venzon, D., Kreitman, R. J., Wayne, A. S., Yuan, C. M., Marti, G. E., & Stetler-Stevenson, M. (2011). Variables affecting the quantitation of CD22 in neoplastic B cells. Cytometry. Part B, Clinical cytometry, 80(2), 83-90.

In certain embodiments, the disease is CDI9+ ALL. In certain embodiments, the tumor cells have a low density of CD 19 on the tumor cells. The method of treatment, or application of the present invention is particularly well suited in patient for whom the targeted antigen is expressed at a low density in at least 50% of the tumor cells, notably 50% of the tumor cells expressing the said antigen.

The cells may be administered at certain doses. For example, the immune cells (e.g., T cells or NK cells) in which SUV39H1 has been inhibited may be administered to adults at doses of less than about 108 cells, less than about 5×10⁷ cells, less than about 10⁷ cells, less than about 5×10⁶ cells, less than about 10⁶ cells, less than about 5×10⁵ cells or less than about 10⁵ cells. The dose for pediatric patients may be about 100-fold less. In alternative embodiments, any of the immune cells (e.g. T-cells) described herein may be administered to patients at doses ranging from about 10⁵ to about 10⁹ cells, or about 10⁵ to about 10⁸ cells, or about 10⁵ to about 10⁷ cells, or about 10⁶ to about 10⁸ cells.

The cancer may be a solid cancer or a “liquid tumor” such as cancers affecting the blood, bone marrow and lymphoid system, also known as tumors of the hematopoietic and lymphoid tissues, which notably include leukemia and lymphoma. Liquid tumors include for example acute myelogenous leukemia (AML), chronic myelogenous leukemia (CML), acute lymphocytic leukemia (ALL), and chronic lymphocytic leukemia (CLL), (including various lymphomas such as mantle cell lymphoma, non-Hodgkins lymphoma (NHL), adenoma, squamous cell carcinoma, laryngeal carcinoma, gallbladder and bile duct cancers, cancers of the retina such as retinoblastoma).

Solid cancers notably include cancers affecting one of the organs selected from the group consisting of colon, rectum, skin, endometrium, lung (including non-small cell lung carcinoma), uterus, bones (such as Osteosarcoma, Chondrosarcomas, Ewing's sarcoma, Fibrosarcomas, Giant cell tumors, Adamantinomas, and Chordomas), liver, kidney, esophagus, stomach, bladder, pancreas, cervix, brain (such as Meningiomas, Glioblastomas, Lower-Grade Astrocytomas, Oligodendrocytomas, Pituitary Tumors, Schwannomas, and Metastatic brain cancers), ovary, breast, head and neck region, testis, prostate and the thyroid gland.

Preferably, a cancer according to the disclosure is a cancer affecting the blood, bone marrow and lymphoid system as described above. Typically the cancer is, or is associated with, multiple myeloma.

In some embodiments, the subject is suffering from or is at risk of an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus.

In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease multiple sclerosis, asthma, and/or a disease or condition associated with transplant.

The present disclosure also relates to a method of treatment and notably an adoptive cell therapy, preferably an adoptive T cell therapy, comprising the administration to a subject in need thereof of a composition a previously described.

In some embodiments, the cells or compositions are administered to the subject, such as a subject having or at risk for a cancer or any one of the diseases as mentioned above. In some aspects, the methods thereby treat, e.g., ameliorate one or more symptom of, the disease or condition, such as with reference to cancer, by lessening tumor burden in a cancer expressing an antigen recognized by the engineered cell.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject. In some embodiments, HLA matching is less important when the immune cell has been modified to reduce expression of endogenous TCR and HLA class I molecules.

Administration of at least one cell according to the disclosure to a subject in need thereof may be combined with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cell populations are administered prior to the one or more additional therapeutic agents. In some embodiments, the cell populations are administered after to the one or more additional therapeutic agents.

With reference to cancer treatment, a combined cancer treatment can include but is not limited to cancer chemotherapeutic agents, cytotoxic agents, hormones, anti-angiogens, radiolabelled compounds, immunotherapy, surgery, cryotherapy, and/or radiotherapy.

Conventional cancer chemotherapeutic agents include alkylating agents, antimetabolites, anthracyclines, topoisomerase inhibitors, microtubule inhibitors and B-raf enzyme inhibitors.

Alkylating agents include the nitrogen mustards (such as mechlorethamine, cyclophosphamide, ifosfamide, melphalan and chlorambucil), ethylenamine and methylenamine derivatives (such as altretamine, thiotepa), alkyl sulfonates (such as busulfan), nitrosoureas (such as carmustine, lomustine, estramustine), triazenes (such as dacarbazine, procarbazine, temozolomide), and platinum-containing antineoplastic agents (such as cisplatin, carboplatin, oxaliplatin).

Antimetabolites include 5-fluorouracil (5-FU), 6-mercaptopurine (6-MP), Capecitabine (Xeloda®), Cytarabine (Ara-C®), Floxuridine, Fludarabine, Gemcitabine (Gemzar®), Hydroxyurea, Methotrexate, Pemetrexed (Alimta®).

Anthracyclines include Daunorubicin, Doxorubicin (Adriamycin®), Epirubicin. Idarubicin. Other anti-tumor antibiotics include Actinomycin-D, Bleomycin, Mitomycin-C, Mitoxantrone.

Topoisomerase inhibitors include Topotecan, Irinotecan (CPT-11), Etoposide (VP-16), Teniposide or Mitoxantrone

Microtubule inhibitors include Estramustine, Ixabepilone, the taxanes (such as Paclitaxel, Docetaxel and Cabazitaxel), and the vinca alkaloids (such as Vinblastine, Vincristine, Vinorelbine, Vindesine and Vinflunine)

B-raf enzyme inhibitors include vemurafenib (Zelboraf), dabrafenib (Tafinlar), and encorafenib (Braftovi)

Immunotherapy includes but is not limited to immune checkpoint modulators (i.e. inhibitors and/or agonists), cytokines, immunomodulating monoclonal antibodies, cancer vaccines.

Preferably, administration of cells in an adoptive T cell therapy according to the disclosure is combined with administration of immune checkpoint modulators. Examples include inhibitors of (e.g. antibodies that bind specifically to and inhibit activity of) PD-1, CTLA4, LAG3, BTLA, OX2R, TIM-3, TIGIT, LAIR-1, PGE2 receptors, and/or EP2/4 Adenosine receptors including A2AR. Preferably, the immune checkpoint modulators comprise anti-PD-1 and/or anti-PDL-1 inhibitors (e.g., anti-PD-1 and/or anti-PDL-1 antibodies).

The present disclosure also relates to the use of a composition comprising the engineered immune cell as herein described for the manufacture of a medicament for treating a cancer, an infectious disease or condition, an autoimmune disease or condition, or an inflammatory disease or condition in a subject.

### EXAMPLES

### Example 1—Inactivating SUV39H1 in Human CD8+ T Cells (SUV39H1 Knockout T Cells)

Activated human CD8+ T cells or progenitors thereof are electroporated with Cas9 ribonucleoprotein particles (RNPs) containing gRNAs that targeted exons of the SUV39H1 gene (SEQ ID NO: 15) for deletion. A consistent decrease in SUV39H1 expression is observed by RT-qPCR four days post electroporation, indicating that the knockout is successful, as shown in Example 1B below.

The memory phenotype of the SUV39H1 KO T cells is evaluated. Cells are stimulated with aCD3+aCD28 beads for one week and then analyzed by flow cytometry. The central memory T cell markers CCR7, CD27 and CD62L showed increased levels of expression in SUV39H1 KO cells. The fraction of CCR7+CD45RO+CD27+CD62L+ cells, which constitute the central memory cell subset, is increased in SUV39H1 KO cells. Increased persistence and reduced exhaustion is evaluated by stimulating the cells once a week for 4 weeks. The SUV39H1 KO cells continue to display increased proliferation after serial stimulations, as shown in Example 2D below.

### Example 1A

In one example experiment, pan CD3+(negative isolation) T cells from 2 healthy donors were rapidly thawed on day 0 and activated in X-vivo T-cell media supplemented with IL-7 (450 U/ml), IL-15 (60 U/ml) and TransAct (1:100). Approximately 24 hours post activation (day 1), T cells were subjected to electroporation with CRISPR-Cas9 ribonucleoprotein particles (RNPs) containing either scrambled (control) gRNA, designated “scr”, or SUV39H1 gRNA targeting exon 2 of the SUV39H1 gene, designated “SUV KO”. The T cells contacted with scrambled gRNA retain functional SUV39H1, while the T cells contacted with SUV39H1-targeting gRNA comprise inactivated SUV39H1. Subsequently, the cells were transduced with vectors to produce modified TCR as described in Example 2A below.

### Example 1B

SUV39H1 protein depletion in HIT T-cells prepared according to Examples 1A and 2A was assessed by Western blot. Protein from transduced T-cells was harvested on the last day of expansion prior to freezing. Cells were washed with PBS with protease inhibitors, lysed with SDS lysis buffer and passed through Qiashredder columns. Lysates were quantified for protein, resuspended in 4× Laemmli buffer in reducing conditions and resolved on 10% protein gels, then subsequently transferred to a nitrocellulose membrane. The membranes were blocked and then incubated with SUV39H1-specific primary antibody or GAPDH loading control antibody overnight. After washing, membranes were incubated with the corresponding HRP-conjugated secondary antibody, washed several times in TBS-T, and developed via application of ECL substrate according to manufacturer's instructions.

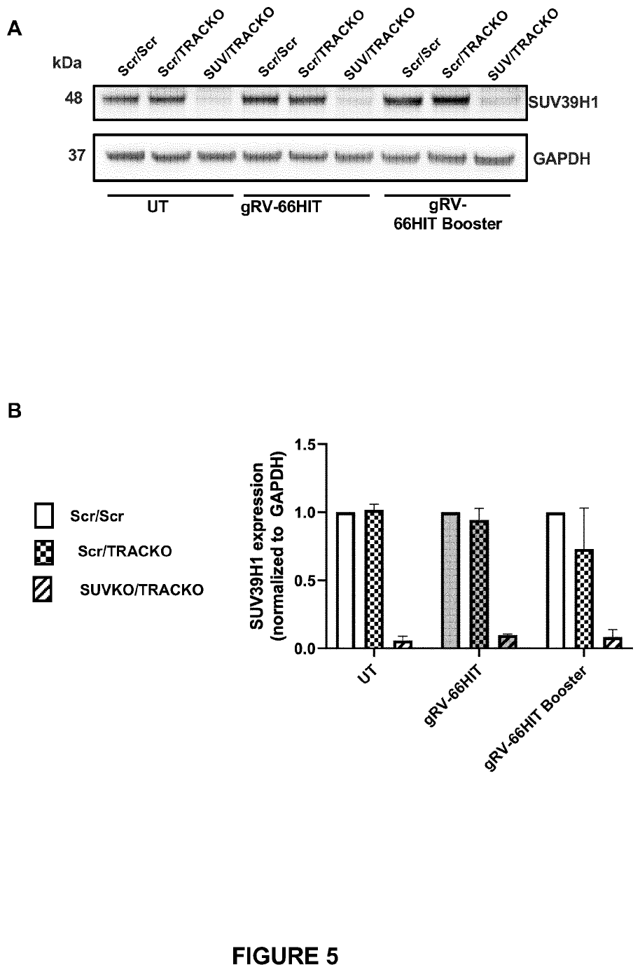

FIG. 5 illustrates that the cells electroporated with SUV39H1-targeting gRNA displayed efficient knockdown of SUV39H1. HiT cells electroporated with a SUV39H1-targeting gRNA showed depletion of SUV39H1 protein levels in contrast to HiT cells electroporated with a scrambled control gRNA (FIG. 5A). An average of 90-95% loss of SUV39H1 protein was observed in SUV39H1 KO T cells (transduced with either HiT or HiTBooster) compared to the scrambled controls (FIG. 5B, mean of two donors). These data show efficient gene editing of SUV39H1 in the HiT/HiTBooster T-cells.

### Example 2—Introduction of Modified TCR

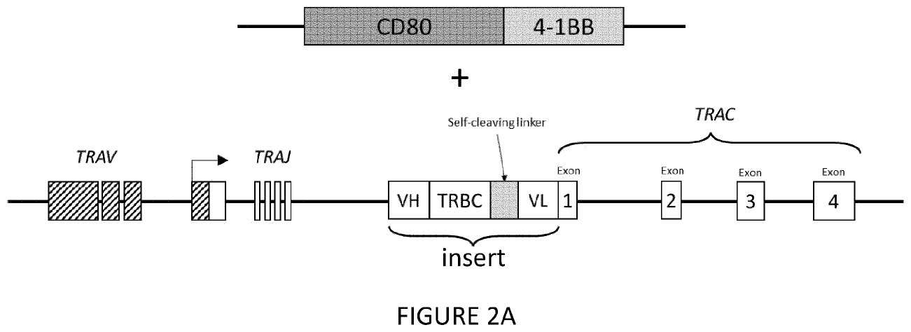

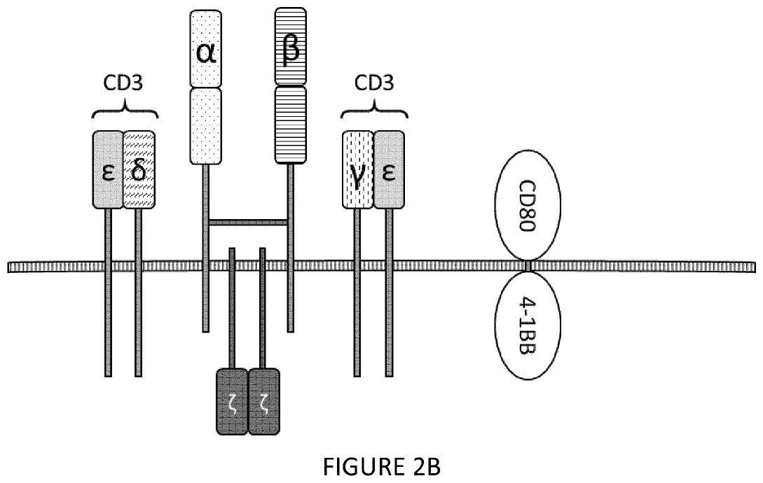

The cells are transduced, either at the same time as SUV39H1 knockout, or sequentially, with a donor viral vector containing the insert of FIG. 2A (VH-TRBC, VL), with homology arms complementary to the adjacent regions of sufficient homology to facilitate integration of the new sequence, e.g., next to TRAC exon 1 (SEQ ID NO: 17). The VH and VL comprise antigen-binding fragments of an antibody that specifically binds, e.g., CD19. CRISPR-Cas9 RNPs were used to introduce the CAR gene into the T-cell receptor α constant (TRAC) locus, resulting in T cells that have significantly reduced or nearly eliminated expression of endogenous TCR, as shown in Eyquem et al., Nature, 543: 113: 117 (2017). The resulting T cells express the CAR under the control of the endogenous TRAC promoter.

The percentage of cells expressing the modified TCR is evaluated, and an appreciable number of cells express modified TCR, as shown in Example 2B below. The expression of endogenous TCR is also evaluated and is reduced.

The properties of the SUV39H1 KO cells expressing modified TCR is evaluated for killing of cells expressing the target antigen, e.g., CD19-positive Raji cells or CD19-positive NALM-6 cells, in vitro and/or in vivo in mice.

T cells that are produced demonstrate both knock-in of the modified TCR in the TRAC locus and specific deletion of SUV39H1.

### Example 2A

In one example experiment, T cells with either (1) functional SUV39H1 gene or (2) inactivated SUV39H1 gene prepared as above were further modified to express HI-TCR specific for mesothelin antigen. On day 2 following the first round of electroporation, T cells received a second round of electroporation with CRISPR-Cas9 ribonucleoprotein particles (RNPs) containing either scrambled (control) gRNA, described “scr”, or TRAC gRNA (targeting the TRAC locus (exon 1 as described in Mansilla-Soto, J., Eyquem, J., Haubner, S. et al. HLA-independent T cell receptors for targeting tumors with low antigen density. Nat Med 28, 345-352 (2022), described “TRAC KO”. Approximately 24 hours later (day 3), T cells were transduced with retroviral particles (donor viral vector) containing (a) the insert of FIG. 2A (encoding VH of anti-mesothelin Ab, TRBC, 2A self-cleaving linker, VL of anti-mesothelin Ab and sufficient exon sequence to reconstitute the TRAC polypeptide, 2A self-cleaving linker, booster sequence (CD80)), designated “RV-66HiT booster” or “HiTBooster”, (b) the insert of FIG. 2A without the booster sequence (VH-TRBC-2A-VL-exon to reconstitute TRAC), designated “RV-66HiT” or “HiT”, or (c) were left alone (for untransduced controls, “UT”) on retronectin coated 6-well plates by spinoculation. On day 4, cells were transferred to a 6-well G-Rex in a total of 30 ml of X-vivo T cell media with IL-7 (450 U/ml) and IL-15 (60 U/ml).

### Example 2B

On day 8, cells were evaluated for transduction efficiency by staining with recombinant mesothelin protein (FITC) and the transduction marker LNGFR (for gRV-66HiT) or CD80 (for gRV-66HiTBooster). The cells were also assessed for reconstitution of CD3 by staining with Pacific Blue rodent anti-human CD3 antibody.

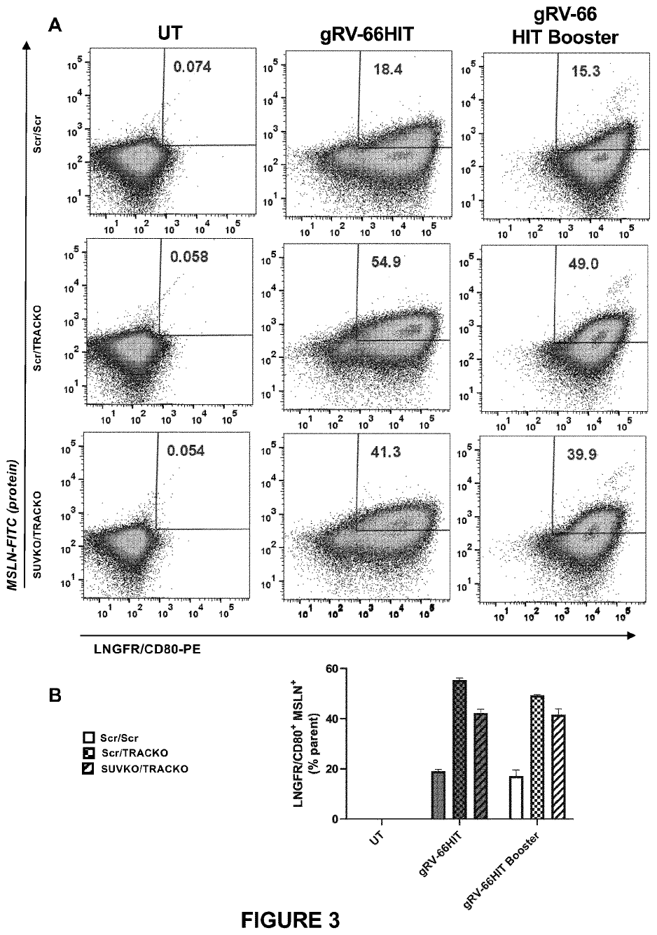

HiT+ cells were defined by binding to recombinant MSLN and staining for expression of LNGFR for HiT or CD80 for HiTBooster (FIG. 3). FIG. 3 illustrates that the transduced T-cells show LNGFR (for gRV-66HIT)/CD80 (for gRV-66HIT Booster) expression and bind to human MSLN-Fc fusion protein. SUV39H1 KO TRAC KO T cells transduced with the HiT construct were approximately 42.3% HiT+(mean of 2 donors) while those transduced with HitBooster construct were 41.5% HiT+ on day 8. This transduction efficiency was comparable to the SUV39H1 WT counterparts (mean of 55.4% for HiT and 49.2% for HiTBooster (CD80) in this experiment (FIG. 3).

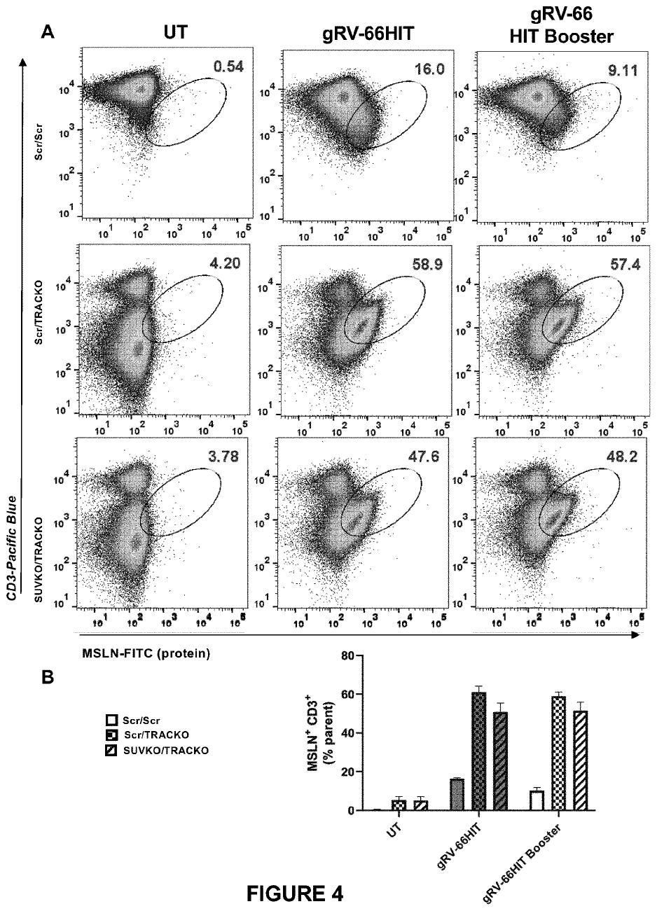

Given that the disruption of the endogenous TRAC locus leads to substantial decrease in cell-surface CD3 expression as shown in FIG. 4A (middle panel and lower panel with untransduced compared to scr/scr control in top panel), an alternative method to define HiT+ T cells was utilized. Reconstitution of CD3 complex expression along with MSLN binding was assessed. Indeed, partial CD3 reconstitution along with concomitant binding to MSLN protein was observed in HiT-transduced cells. FIG. 4 illustrates that the transduced T-cells show both CD3 complex reconstitution and surface binding to recombinant mesothelin. Specifically, SUV39H1 KO TRAC KO T cells transduced with the HiT construct were approximately 50.8% HiT+(mean of 2 donors) while those transduced with HitBooster construct (CD80) were 51.4% for HiTBooster T-cells on day 8. This transduction efficiency was comparable to the SUV39H1 WT counterparts (mean of 61% for HiT and 58.9% for HiTBooster) (FIG. 4B).

### Example 2C: Cytotoxic Activity

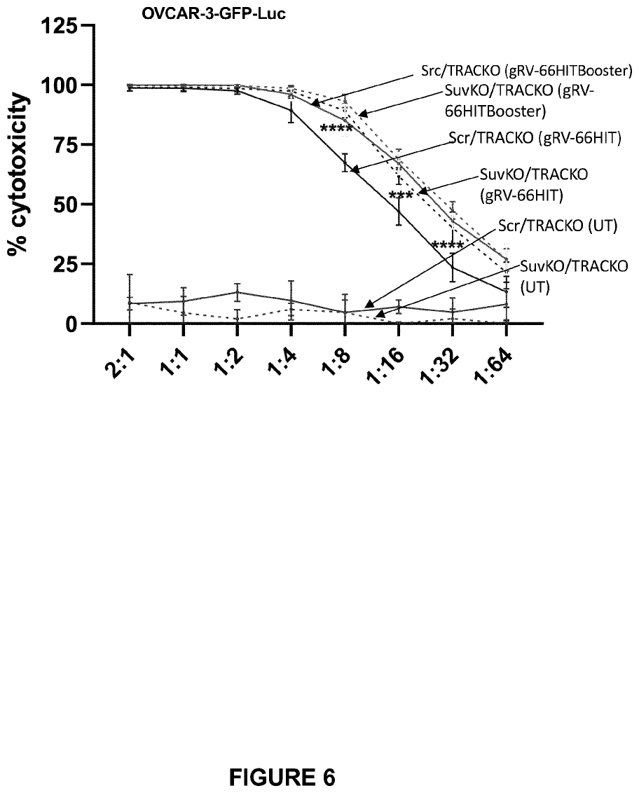

A luciferase-based assay was used to evaluate the cytotoxic ability of anti-MSLN HiT+ cells against MSLN+ and MSLN− target cells. Target cells were seeded at a density of 1×104 cells/well in a 96-well plate with HiT+ effector T cells at E:T ratios ranging from 2:1 to 1:64 at 2-fold dilutions in a total volume of 200 uL. Target cell kill was assessed 48 hours post seeding of HiT T-cells. HiT+ T cells from both donors mediated lysis of MSLN+ OVCAR3 tumor cells (FIG. 6). Cytotoxicity was dose dependent, with SUV39H1 KO HiT+ T cells showing markedly improved cytotoxic function at the lower E:T ratios of 1:8, 1:16 and 1:32.

Additionally, the SUV39H1 KO HiT+ cells without the booster exhibited comparable cytotoxic capacity with HiTBooster+ T-cells with or without SUV39H1. Importantly, this cytotoxicity was specific to the presence of target expression as the MSLN-line HEK293T cells did not induce a response from HiT+ cells (SUV39H1 KO or WT, in the presence or absence of booster). Remarkably, this data indicates that the SUV39H1 inactivation improved immune cell cytotoxic activity to the same extent as the chimeric booster receptor.

### Example 2D: Repeat Stimulation Assay

To analyze T cell proliferation and persistence, SUV39H1 KO and control HiT+ cells were stimulated with MSLN+ target cell lines at an E:T ratio of 1:2. Following 3 days of initial stimulation, HiT-Booster+ cell numbers were enumerated using counting beads and anti-LNGFR or CD80 specific antibodies by flow cytometry. Transduced T cells were also stained for antibodies specific for CD4, CD8, CCR7, CD27 and CD45RO prior to analysis via flow cytometry. Cells were also stained with CD33 to exclude target cells. Subsequently, T cells were restimulated under the same conditions every 3-4 days. These steps were repeated up to day 36 post initial stimulation. To address the persistence and functionality of SUV deficient HiT-Booster cells, both WT and SUV39H1 KO HiT-Booster T cells were exposed to repeated antigen exposure with Nomo-1 target cells, an endogenously expressing MSLN+ target line.

On day 8 (FIG. 9A), SUV39H1 WT HiT-Booster cells showed marginally increased expansion when compared to SUV39H1 KO HiT-Booster cells. However, by day 19, SUV39H1 KO HiT-Booster cells proliferated more than SUV39H1 WT cells and continued to expand at later time points such as day 22 & 26. Additionally, by day 36, SUV39H1 KO HiT-Booster cells continued to suppress MSLN+ target cell outgrowth in contrast to SUV39H1 WT HiT-Booster cells (FIG. 9B). SUV39H1 KO HiT-Booster cells also showed an enrichment in the proportion of CD27+CD45RO+ as well as CCR7+CD45RO+ memory cells, demonstrating an increase in the central memory HiT-Booster+ T cell populations (FIGS. 9C and 9D). This enhanced memory phenotype was observed both prior to (Day 0) and at various timepoints post stimulation (Day 15 and 22) with MSLN+ target cells. Persistence of CD8+ HiT-Booster+ cells was tracked over repeated rounds of stimulation. At later time points (Day 26 onwards), SUV39H1 KO HiT-Booster+ cells showed increased CD8/CD4 ratio (FIG. 9E), suggesting preferential persistence of CD8+ HiT-Booster+ T cells upon depletion of SUV39H1.

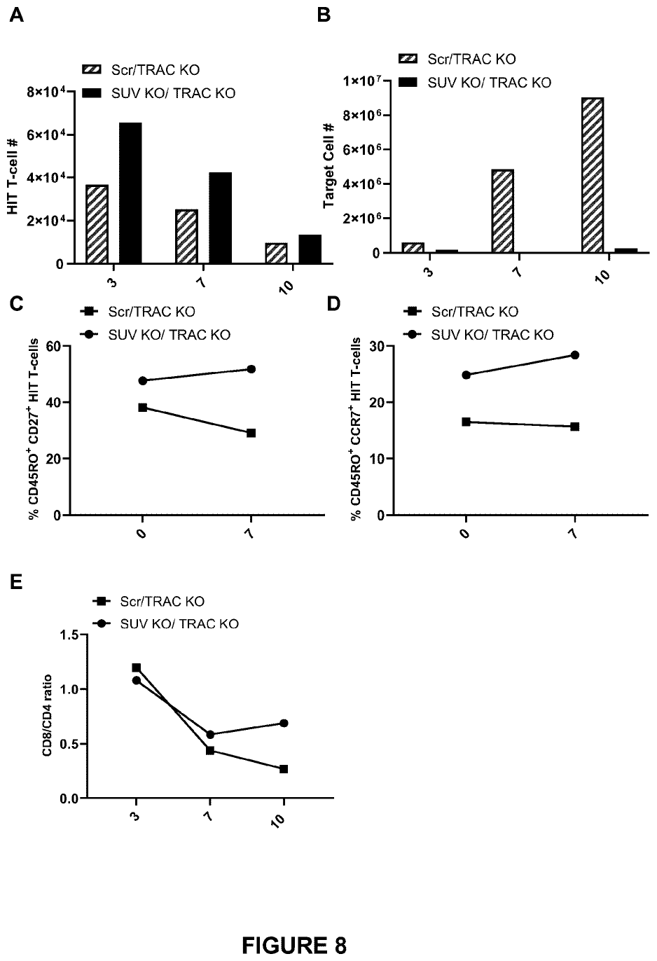

Repeated experiments with an independent donor (FIG. 8) showed consistent trends in increased T-cell proliferation and target killing along with enhanced memory phenotype and persistence of CD8+HiTBooster+ cells.

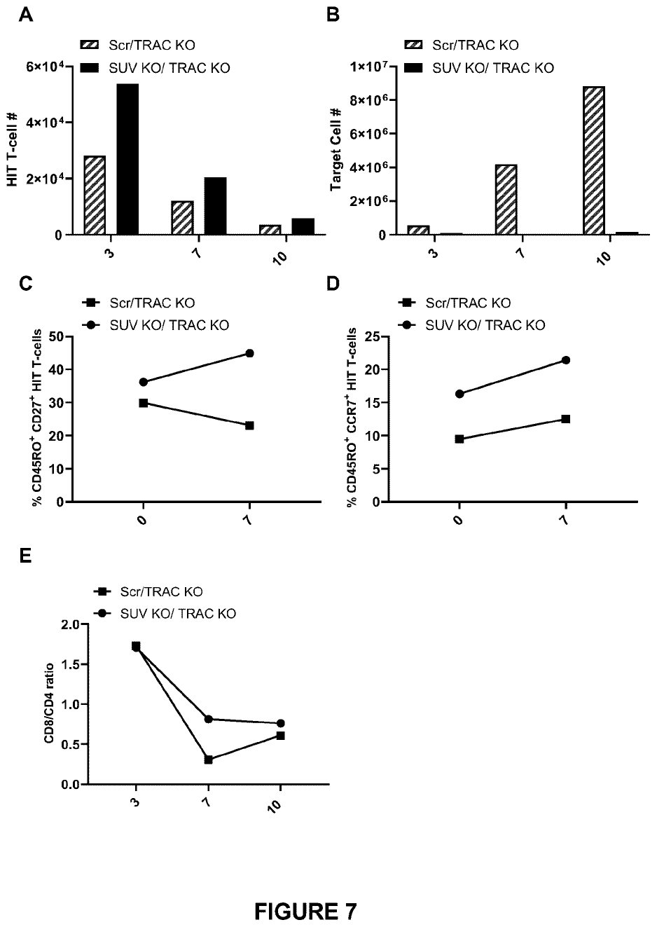

FIG. 7 also illustrates that SUV39H1 depleted gRV-66HIT cells show increased T-cell expansion, persistence, memory, and target killing capacity on repeated antigen exposure with NOMO-1 target lines as compared to wt (src gSUV) gRV-66HIT cells.

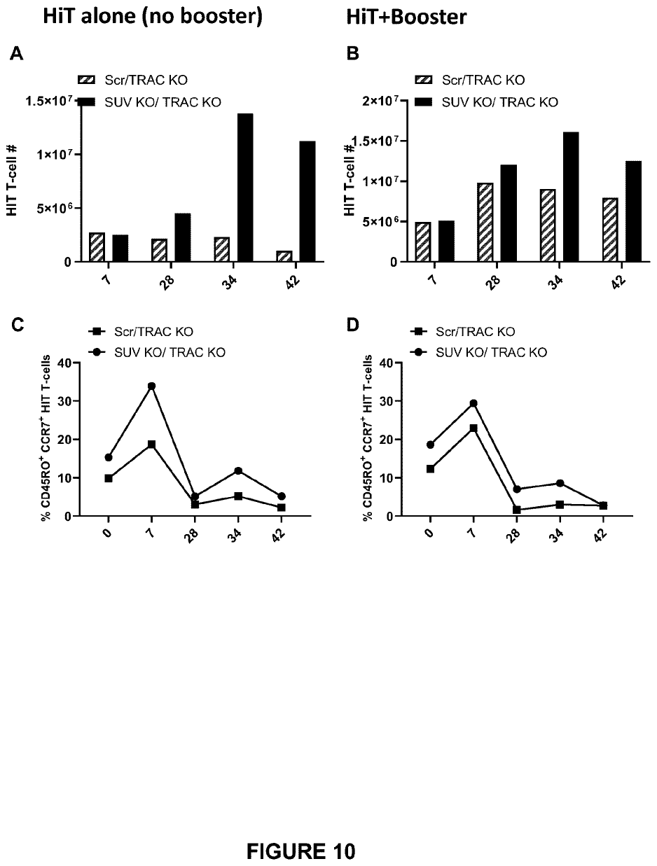

These results with gRV-66HiT and with gRV-66HiTBooster were again confirmed for another donor (FIG. 10).

Together, these results demonstrate that SUV39H1 KO HiT+ cells (in presence or absence of a Booster construct) show better persistence, killing, and memory than their WT counterparts.

### Examples 3-4: Production and Validation of Gammaretroviral (gRV) HITs Constructs Targeting PSMA (mJ591 HIT) or CD19 (FMC63 HIT) for Random Integration

On day 2, cells were electroporated using a Lonza nucleofector using CRISPR/Cas9 to KO SUV39H1 and TRAC in a single step. On day 3 T cells were transduced with gamma retroviruses encoding for the FMC63 HIT, mJ591 HIT, FMC63 HIT+Booster and mJ591 HIT+booster (CD80_4-1BB)

Schematic representations of gammaretroviral (gRV) HIT constructs used herein are shown in FIG. 11. Construct A includes the transduction marker LNGFR. Construct B includes a booster molecule.

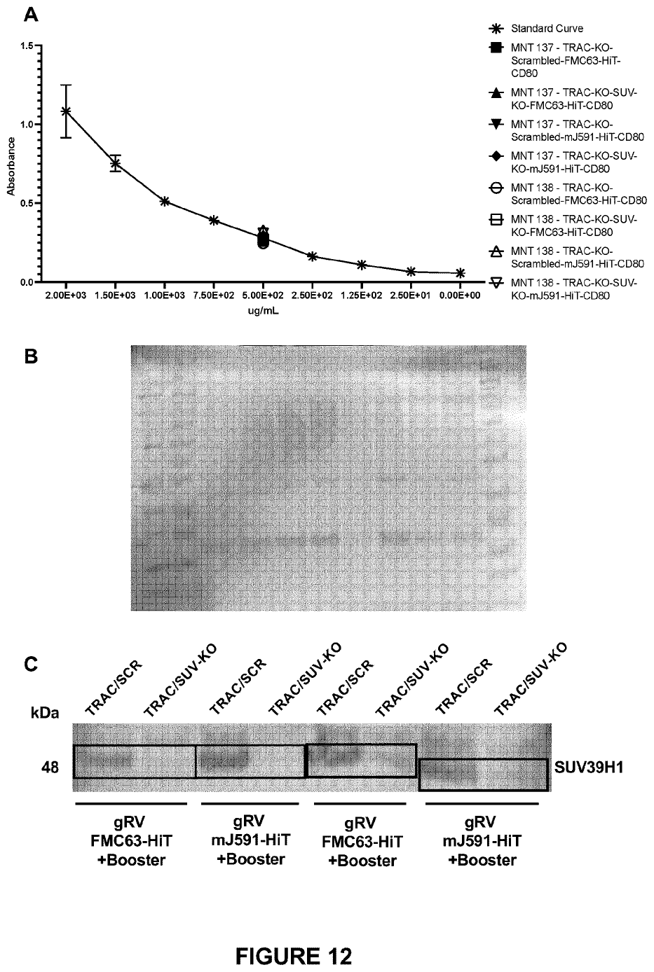

Western blot of SUV39H1 confirmed effective SUV-KO (see FIG. 12) on representative samples of FMC63-HiT+Booster and mJ591-HiT+Booster (CD80_4-1BB) engineered T cells. Western Blot (FIG. 12C) confirms knockout of SUV39H1 band (lower band, highlighted in black box) at 48 kDa.

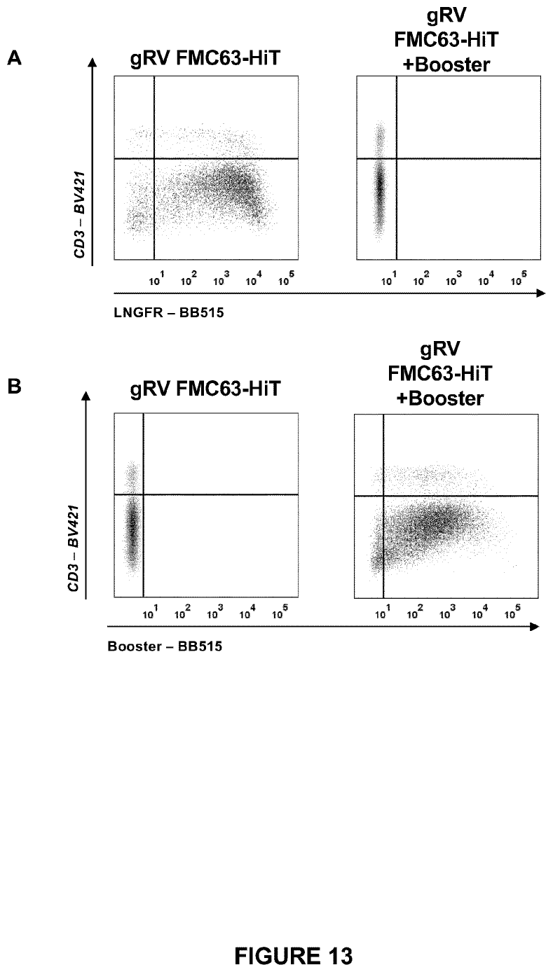

T cells were transduced with gRV-FMC63-HiT and gRV-FMC63-HiT+Booster constructs (FIG. 13). Dot plots show gRV-FMC63-HiT (left) and gRV-FMC63-HiT+Booster cells when transduced with their respective virus based on either LNGFR or booster expression (FIG. 13A). Successful TRAC-KO can be observed with the loss of CD3 when compared to the expression markers (FIG. 13B, similar CD3+ population reduction as in FIG. 4).

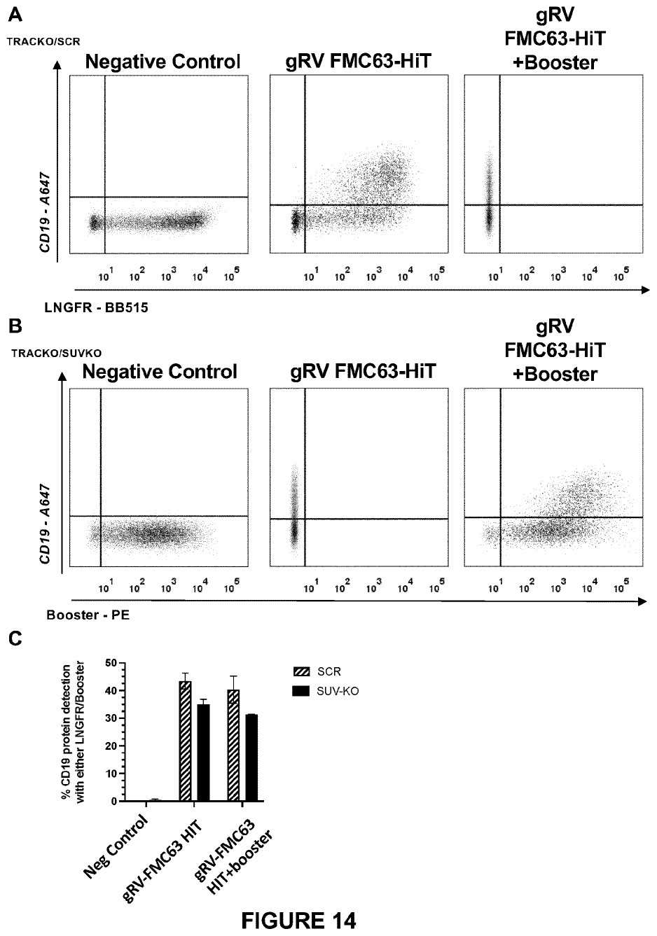

HIT T cells expressing the FMC63 HIT construct specifically bound recombinant CD19 protein (see FIG. 14). TRACKO/Scrambled (SCR) and TRACKO/SUVKO samples were incubated with recombinant biotinylated CD19 protein and stained with streptavidin-A647. Both gRV-FMC63-HiT and gRV-FMC63-HiT+Booster showed similar binding to the recombinant CD19 protein in combination with their respective expression marker (LNGFR and Booster).

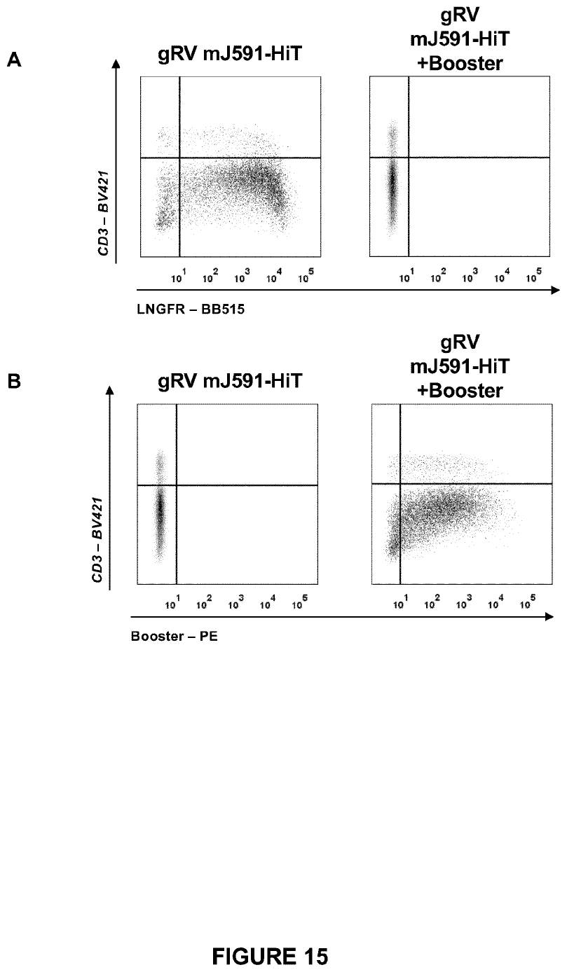

T cells were transduced with gRV-mJ591-HiT (left) and gRV-mJ591-HiT+Booster constructs (FIG. 15). (A) Dot plots show gRV-mJ591-HiT (left) and gRV-mJ591-HiT+Booster cells when transduced with their respective virus based on either LNGFR or booster expression. (B) Successful TRAC-KO was observed with the loss of CD3 when compared to the expression markers (similar CD3+ population reduction as in FIG. 4).

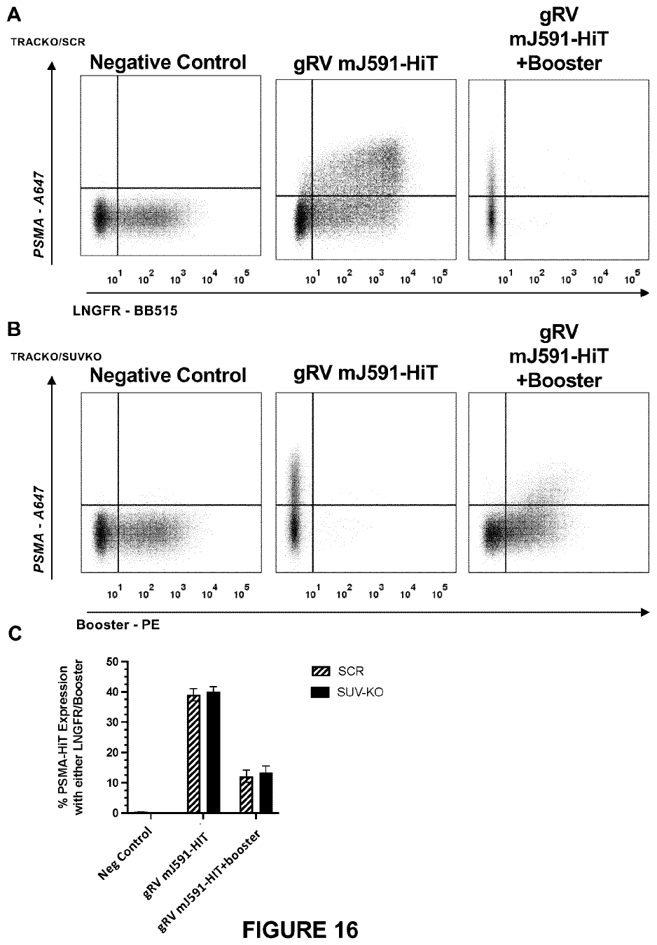

mJ591-HiT T cells specifically bound recombinant PSMA protein (FIG. 16) (A) TRACKO/Scrambled (SCR) and (B) TRACKO/SUVKO samples were incubated with recombinant biotinylated PSMA protein and stained with streptavidin-A647.

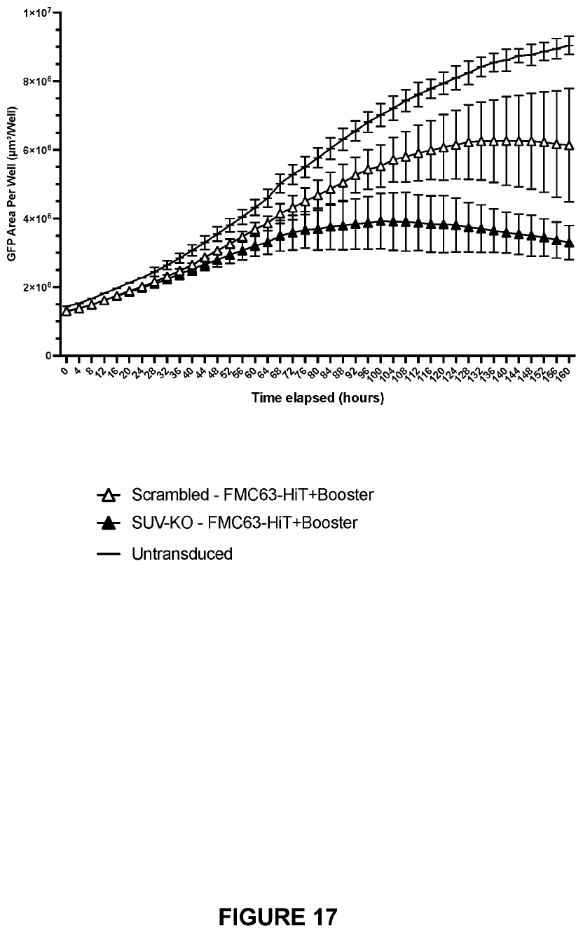

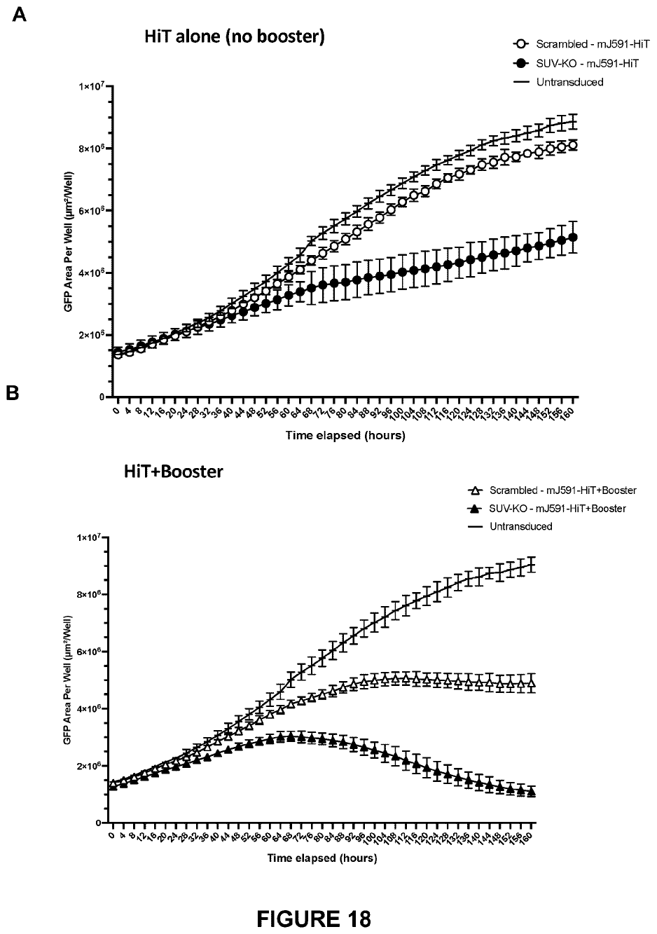

SUV-KO HiT samples exhibited increased killing compared to scrambled (SCR) controls in kinetic kill assay. TRAC-KO-Scramble and TRAC-KO-SUV-KO or gRV-FMC63-HiT+Booster cells were co-cultured with GFP+ LNCaP-19 target line at E:T of 1:20 in 96-well plates and placed into the InCucyte for GFP detection for 7 days. Untransduced T cells were used as negative controls (FIG. 17). TRAC-KO-Scramble and TRAC-KO-SUV-KO (FIG. 18 A) gRV-mJ591-HiT or (FIG. 18B) gRV-mJ591-HiT+Booster cells were co-cultured with GFP+ LNCaP-19 target line at a E:T ratio of 1:20 in a 96-wellplate and placed into the InCucyte for GFP detection for 7 days. Untransduced T cells were used as negative controls.

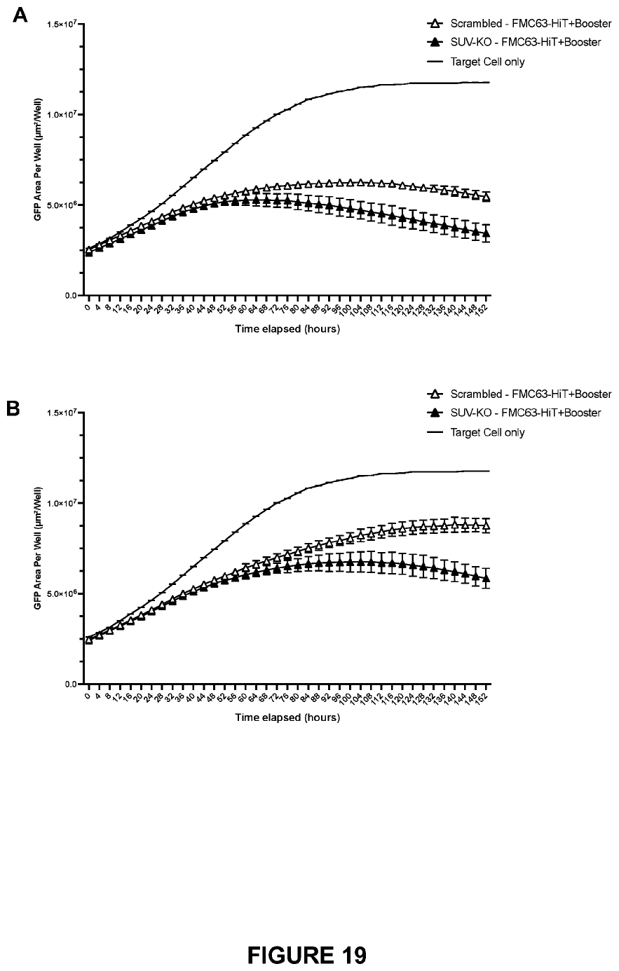

SUV-KO cells show Increased killing compared to scrambled (SCR) controls after a 2^{nd} stimulation in a kinetic killing assay (FIG. 19) TRAC-KO-Scrambled and TRAC-KO-SUV-KO gRV-FMC63-HiT cells having already undergone a 1^{st} antigen stimulation, were filtered with a 20-μM filter and were FACS analyzed to determine % of HiT+ cells. These gRV-FMC63-HiT+Booster cells were co-cultured with GFP+ LNCap-19 target cells at two different E:T ratios: (A) 1:5 and (B) 1:10 in a 96-well plate and placed into the InCucyte for GFP detection for 7 days. “Target cell only” was used as the negative control for these experiments.

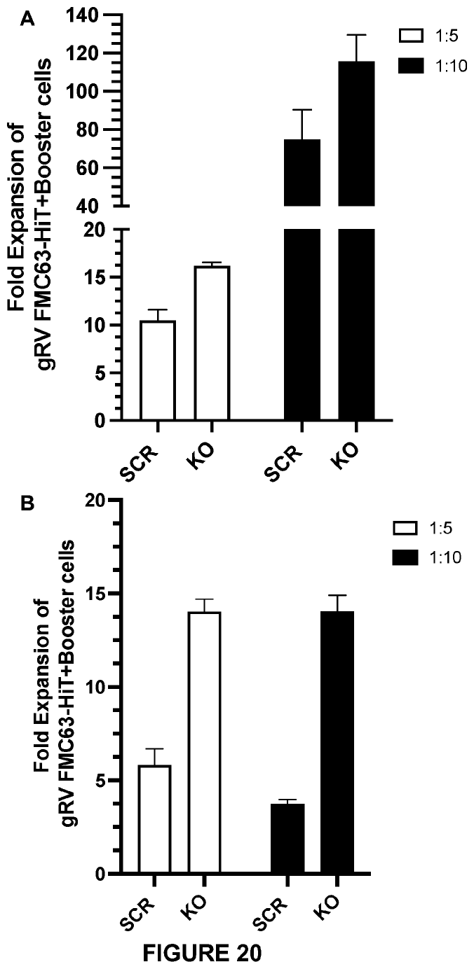

TRAC-KO-SUV-KO gRV FMC63-HiT+Booster T cells exhibit an increased expansion as compared to Scrambled (SCR) controls (FIG. 20). TRAC-KO-Scramble and TRAC-KO-SUV-KO gRV-FMC63-HiT+Booster cells were co-cultured with GFP+ LNCap-19 target cells at the indicated E:T ratios in a 96-well plate and placed into the InCucyte for GFP detection for 7 days. After 7 days, co-cultures underwent FACS analysis for cell counting.

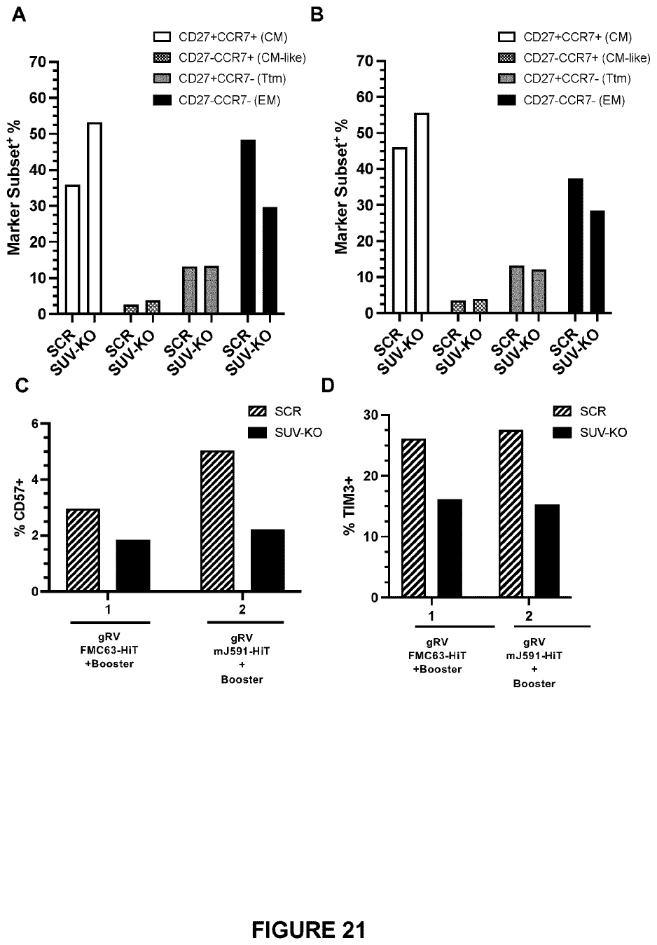

SUV-KO gRV HiT cells exhibit an improved memory-associated profile, reduced effector-like profile and reduced exhaustion marker phenotype compared to Scrambled (SCR) controls after manufacturing (FIG. 21). (A) Phenotype at day 15 after initial T cell activation of gRV TRAC-KO and Scrambled/SUV-KO FMC63-HiT+Booster and (B) gRV mJ591-HiT+booster T cells. Cells were analyzed for surface marker expression under the following gating strategy: (A) and (B) lymphocyte⁺singlet⁺alive⁺CD4^{+/-}CD8^{+/-}Booster⁺CD45RO⁺. CCR7 and CD27 expression were analyzed to check for stemness and broken down accordingly. CM: central memory, Ttm: transitional memory and EM: effector memory. (C) and (D) lymphocyte⁺singlet⁺alive⁺CD4^{+/-}CD8^{+/-}Booster⁺ for CD57 and TIM3 respectively.

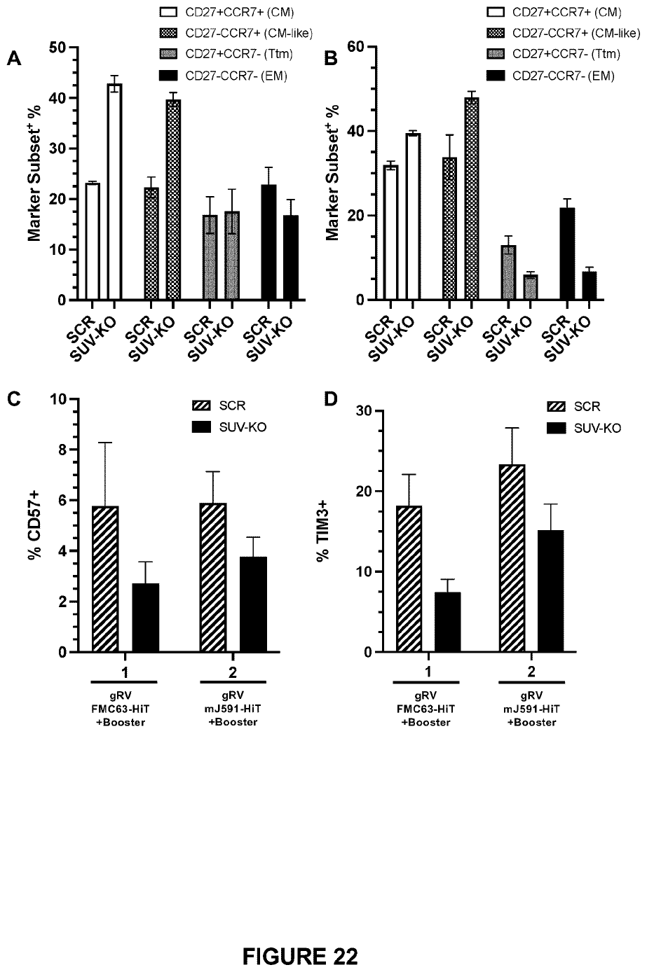

SUV-KO gRV HiT cells exhibit an improved memory-associated profile, reduced effector-like profile and reduced exhaustion marker phenotype after 1st antigen stimulation as compared to Scrambled (SCR) controls (FIG. 22). (A) gRV TRAC-KO and Scrambled/SUV-KO FMC63-HiT+Booster and (B) gRV mJ591-HiT+booster were co-cultured with GFP+ LNCap-19 target line at various E:T ratios. Analysis at day 7 after plating for surface marker expression under the following gating strategy: (A) and (B) lymphocyte⁺singlet⁺alive⁺CD4^{+/-}CD8^{+/-}Booster⁺CD45RO⁺. CCR7 and CD27 expression were analyzed to check for stemness and broken down accordingly. CM: central memory, Ttm: transitional memory and EM: effector memory. (C) and (D) lymphocyte⁺singlet⁺alive⁺CD4^{+/-}CD8^{+/-}Booster⁺ for CD57 and TIM3 respectively.

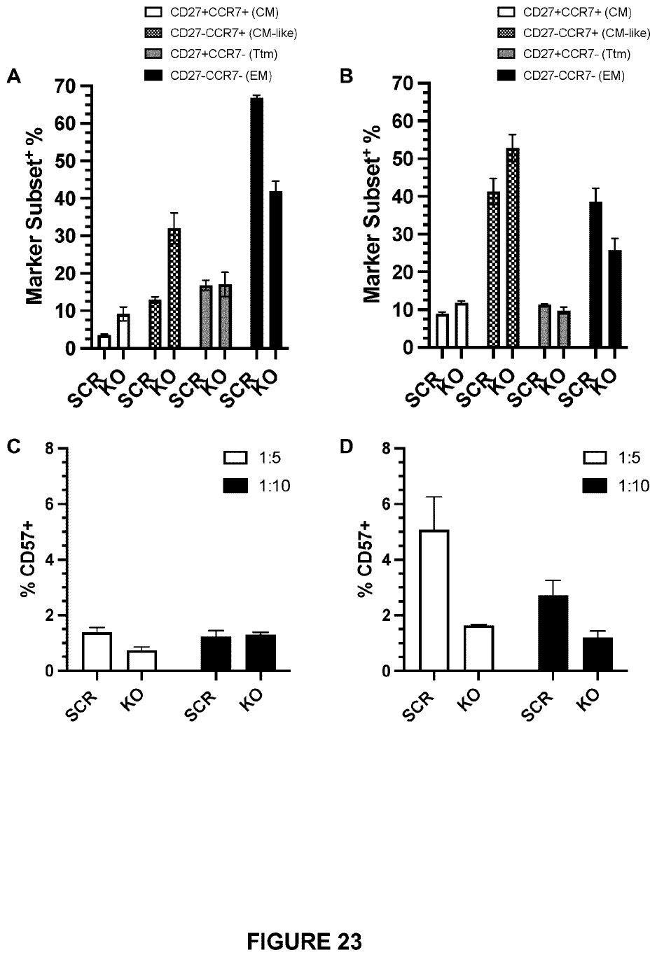

SUV-KO gRV HiT cells exhibit an improved memory-associated profile, reduced effector-like profile and reduced exhaustion marker phenotype after 2nd antigen stimulation compared to Scrambled (SCR) controls (FIG. 23). TRAC-KO-Scrambled and TRAC-KO-SUV-KO gRV-HiT+booster cells, having already undergone a 1st antigen stimulation, were filtered with a 20-μM filter and were FACS analyzed to determine % of HiT+ cells. These gRV-HiT+Booster cells were co-cultured with GFP+ LNCap-19 target line at a E:T ratio of 1:5 (1.5e³ HIT T-cells and 7.5e³ target cells) in a 96-well plate and placed into the InCucyte for GFP detection for 7 days. (A) and (C) graphs display gRV FMC63-HiT+Booster cells and (B) and (D) graphs display gRV mJ591-HiT+booster cells. Analysis at day 7 after plating for surface marker expression under the following gating strategy: (A) and (B) lymphocyte⁺singlet⁺alive⁺CD4^{+/-}CD8^{+/-}Booster⁺CD45RO⁺. CCR7 and CD27 expression were analyzed to check for stemness and broken down accordingly. CM: central memory, Ttm: transitional memory and EM: effector memory. (C) and (D) lymphocyte⁺singlet⁺alive⁺CD4^{+/-}CD8^{+/-}Booster⁺ for CD57 and TIM3 respectively).

### Example 5: Generation of TRAC-HIT Cells with Inactivated SUV39H1

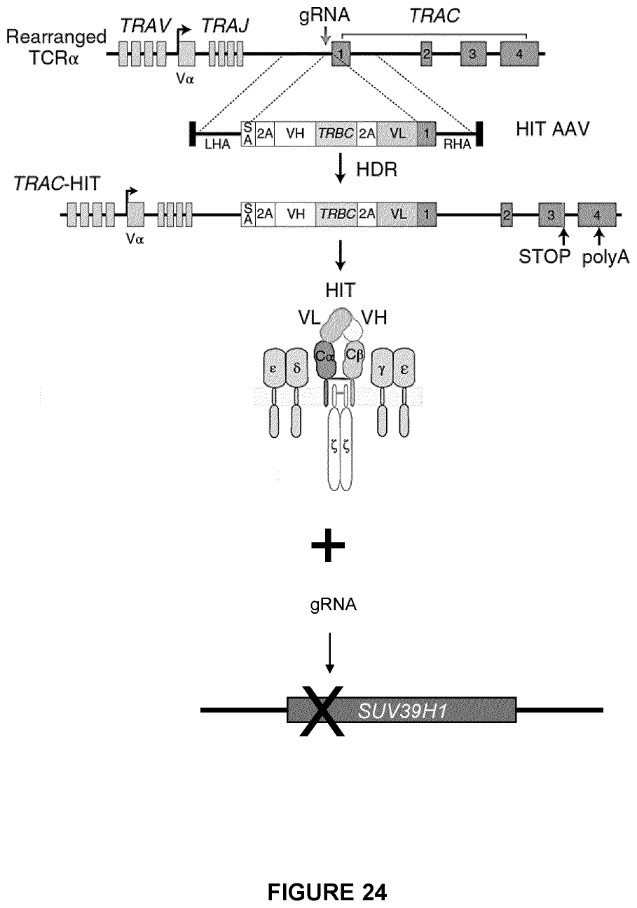

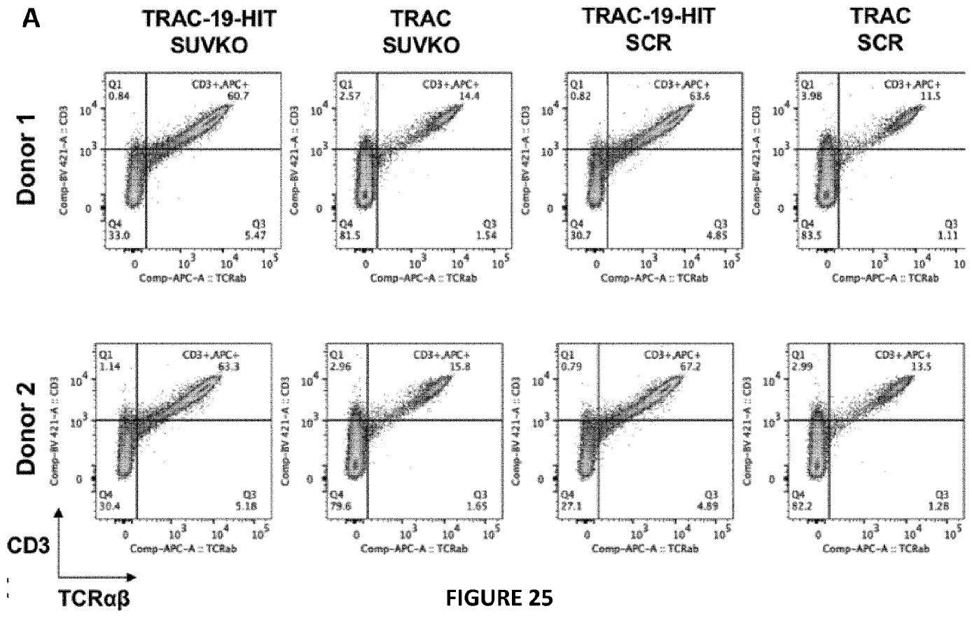

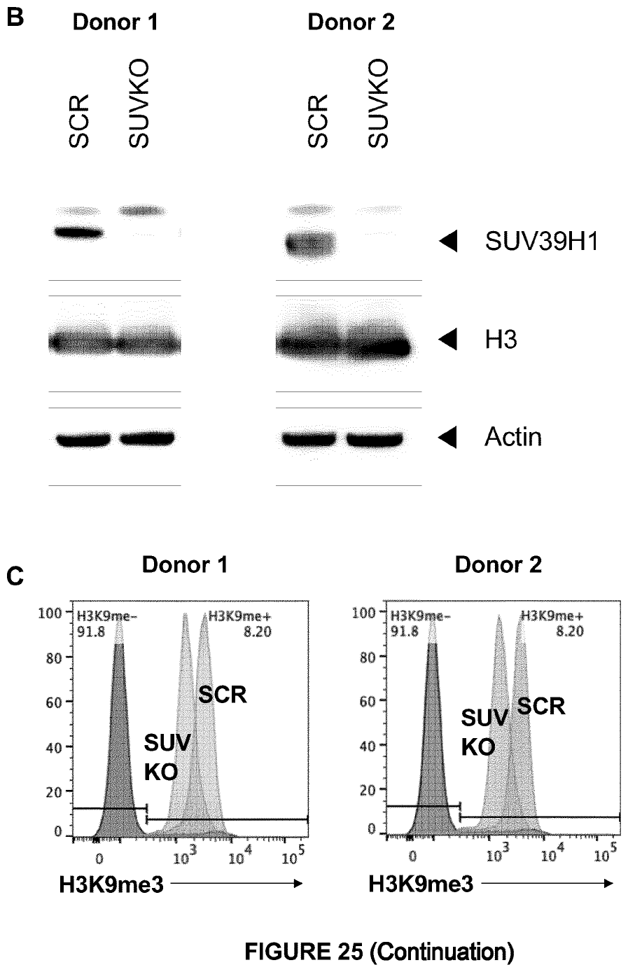

Another approach for the generation of HIT cells uses the approach of knock-in to the TRAC locus. Briefly, an adeno-associated virus (HIT-AAV) vector repair matrix comprising left and right homology arms was designed (Mansilla-Soto et al, Nat Med 2022 February; 28(2):345-352). This targeting construct (HIT-AAV) contains a splice acceptor, followed by P2A cleaving peptide and the CD19-specific HIT gene elements (a VH-Cβ gene followed by P2A and the VL gene), which are joined to the TRAC exon1; all flanked by sequences homologous to the TRAC locus (left and right homology arm: LHA and RHA). In the presence of Cas9 ribonucleoparticles that target the TRAC locus and mediate double strand breaks, the DNA repair machinery will integrate the HIT-AAV matrix. Once integrated, the VH-Cβ and VL-Cα expression is driven by the endogenous TCRα promoter and polyA elements, while endogenous TCRα expression is disrupted (FIG. 24). VH-Cβ and VL-Cα chains will associate to form the HIT heterodimer, which is confirmed by CD3 expression at the cell surface. The approach can be multiplexed by using additional Cas9 RNPs targeting other loci, namely SUV39H1 (FIG. 24). TRAC-HIT cells targeting CD19 and deficient for SUV39H1 were generated from two different donors (FIG. 25A). In the presence of the 19-HIT-AAV repair matrix, successful reconstitution was detected, as seen by the double positive CD3+TCRαβ+ populations. TRAC-19-HIT-SUVKO T cells had lower levels of SUV39H1 protein (FIG. 25B) and lower levels of H3K9me3 (FIG. 25C).

Therefore, TRAC-19-HIT T cells either sufficient (SCR) or deficient for SUV39H1 (SUVKO) were successfully generated and were then cryopreserved for future use.

### Example 6: SUV39H1 KO HIT T Cells have Enhanced Memory Phenotype In Vitro

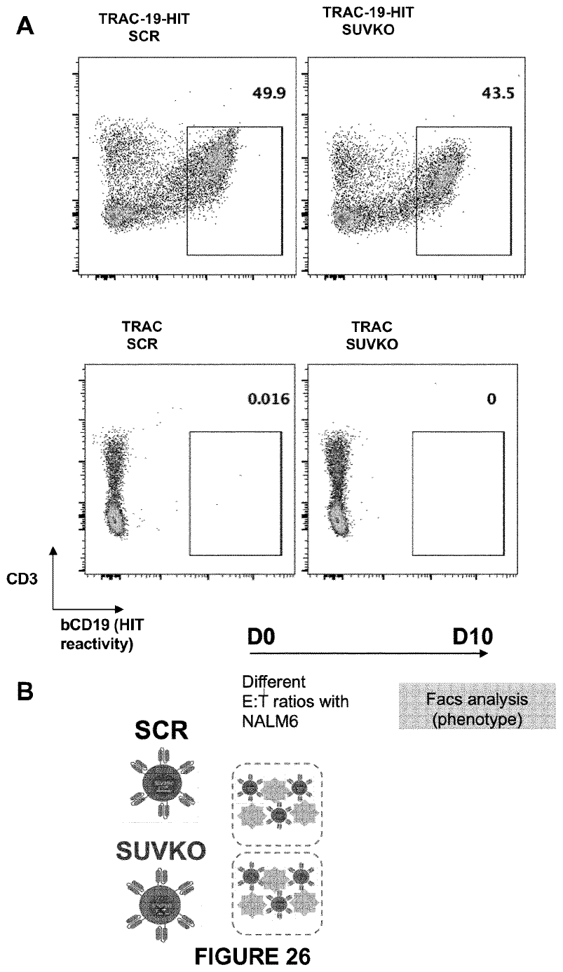

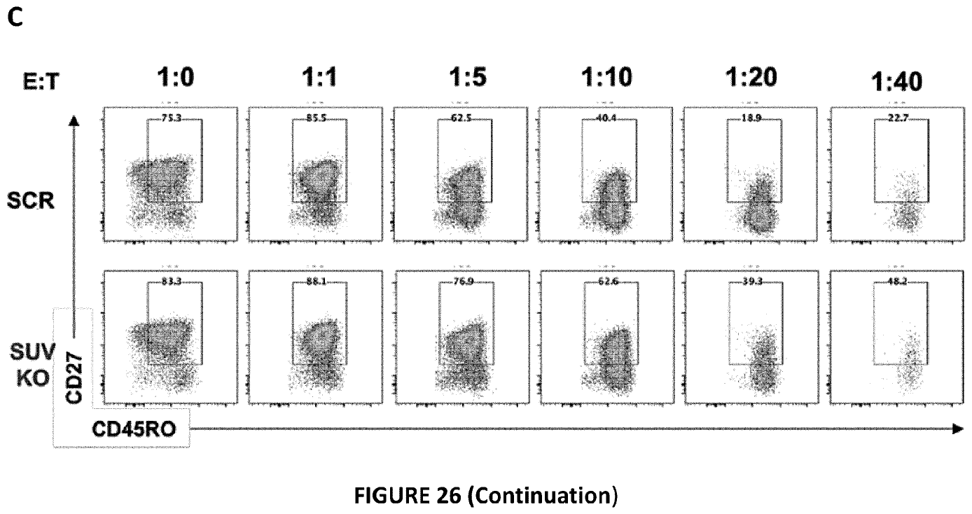

TRAC-19-HIT either sufficient (SCR) or deficient for SUV39H1 (SUVKO) were thawed at 37° C. for 2 hours in culture medium. The cells were then tested for viability and HIT expression. To determine HIT expression at the cell surface, a biotinylated CD19 soluble molecule was used. Both SCR and SUVKO T cells showed good viability and reactivity with soluble CD19 (FIG. 26A). TRAC-19-HIT T cells were then used in an in vitro co-culture assay with NALM6 cells at different effector:target cell ratios (effectors determined by percentage of CD19 reactivity) for ten days (FIG. 26B). After the end of the assay, TRAC-19-HIT cells were phenotyped by flow cytometry. TRAC-19-HIT-SUVKO T cells showed an increase in the percentage of CD27+ cells compared to TRAC-19-HIT-SCR T cells (FIG. 26C).

Therefore, SUV39H1 inactivation enhances the memory phenotype of TRAC-19-HIT cells.

### Example 7: SUV39H1 KO HIT T Cells Better Reject Liquid Tumors

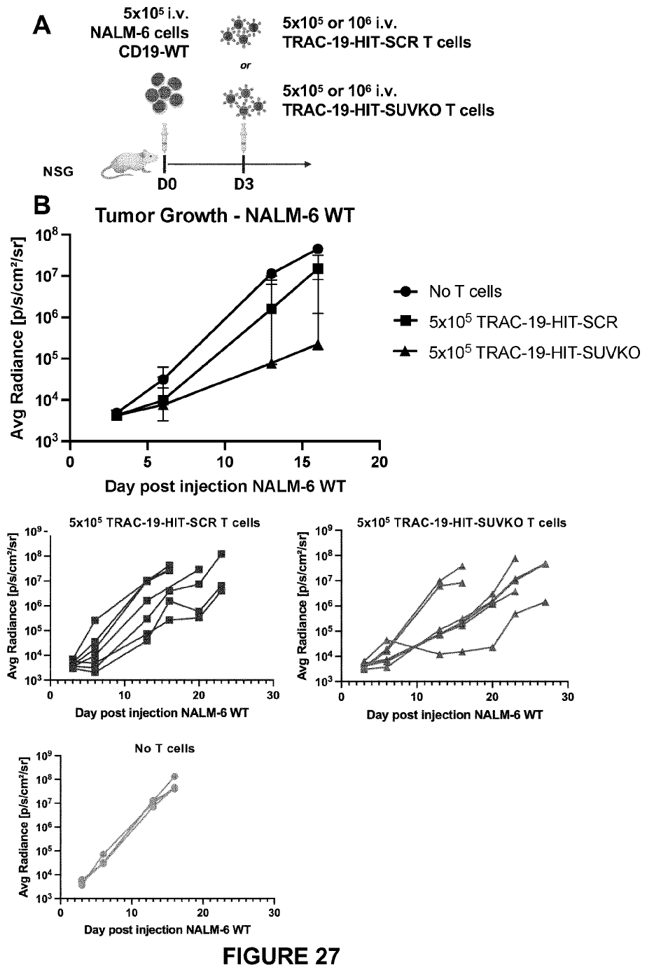

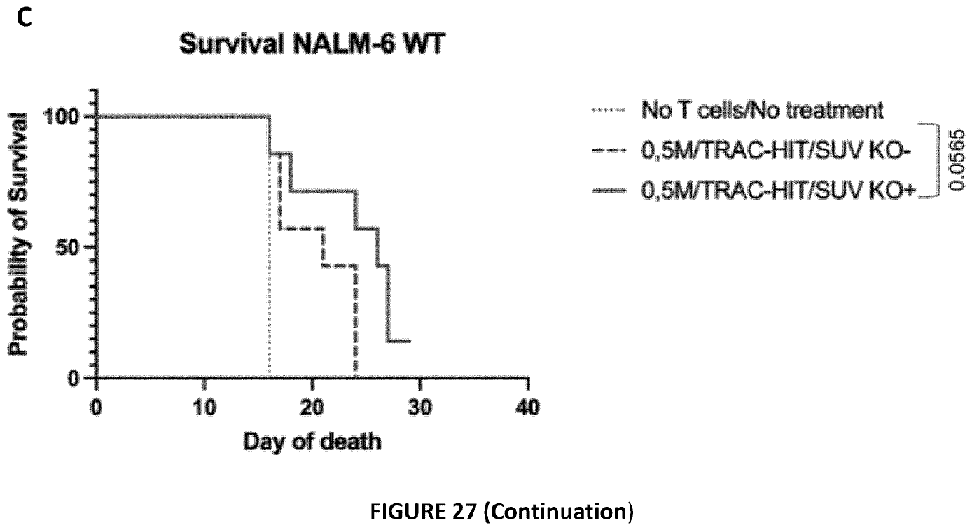

To investigate the effect of SUV39H1 inactivation on HIT T cell antitumor efficacy at different levels of antigen, a xenogeneic model of Acute Lymphoblastic Leukemia (ALL) in NSG mice was used. Briefly, female NSG mice were injected intravenously in the tail on DO with 5×10⁵ NALM6-Luc cells expressing either wild-type levels (20000 molecules per cell) of CD19 (WT) or low levels (200 molecules per cell) of CD19 (LOW) (Mansilla-Soto et al, Nat Med 2022 February; 28(2):345-352). On D3, tumor burden was measured by bioluminescence imaging using Xenogen IVIS Imaging System (PerkinElmer). Acquired bioluminescence data was analyzed using Living Image software (PerkinElmer) and expressed in average radiance (photons/sec/cm2/sr), and the mice were randomized in groups. On the same day, TRAC-19-HIT T cells either sufficient (SCR) or deficient for SUV39H1 (SUVKO) were were injected intravenously in the tail (FIG. 27A). Tumor burden was then assessed one or two times per week.

At a dose of 5×10⁵ cells, TRAC-19-HIT-SUVKO T cells better rejected NALM6-WT cells than TRAC-19-HIT-SCR T cells (FIG. 27B) and increased mouse survival (FIG. 27C).

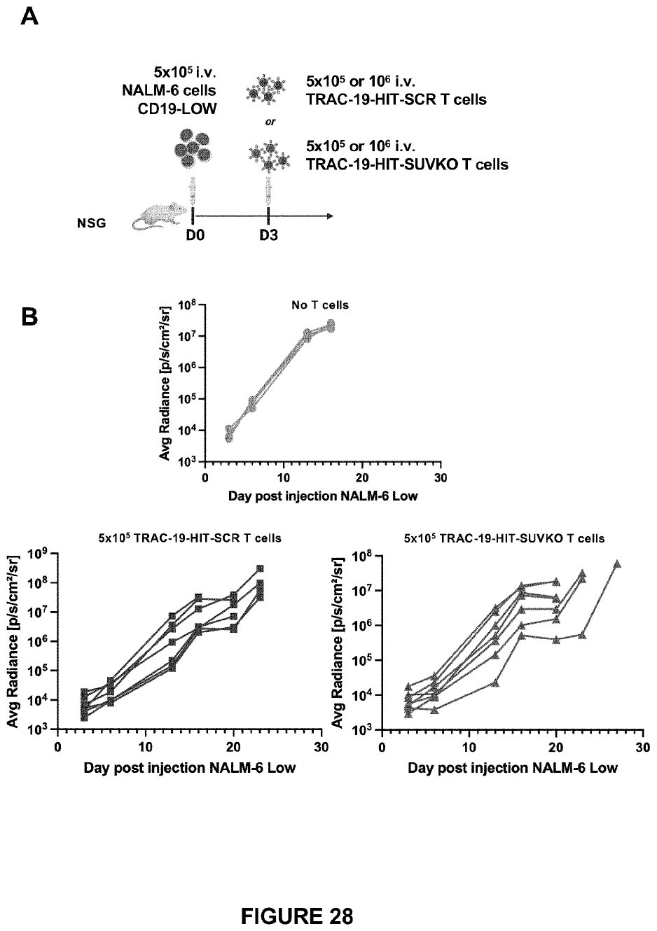

FIG. 28 shows the response of 5×10⁵ TRAC-19-HIT cells against NALM6-LOW cells. SUVKO cells showed a slightly stronger rejection of NALM6-LOW cells in vivo (FIG. 28B right panel). Therefore, SUV39H1 inactivation enhances antitumor function of TRAC-19-HIT T cells against liquid tumors even at low antigen density.

Together the results provided herein demonstrates that SUV39H1 deficiency (e.g., Suv KO) is unexpectedly improving in vivo persistence, killing, and memory phenotype of Hi-TCR T-cells even in the absence of a CCR (booster) receptor.

### Example 8—Cells with Inactivated SUV39H1 and Reduced ITAM Activity

The CD3zeta polypeptide is modified to delete ITAM2 and ITAM3, at the same time as the SUV39H1 knockout, or sequentially, with CRISPR-Cas9 RNPs targeting the gene encoding amino acids 90-164 of CD3zeta (SEQ ID NO: 7) for deletion. The resulting cells are evaluated for their Central Memory Cell phenotype (CCR7+CD45RO+CD27+CD62L+), proliferation after serial stimulation, and exhaustion characteristics (TIM-3, PD-1, LAG-3 expression). The addition of 1XX provides further improvements in cytotoxic activity.

### Sequences:

SEQ ID NO:1: Homo sapiens T cell receptor alpha delta locus (TCRA/TCRD) on chromosome 14
SEQ ID NO:2-3: Homo sapiens T cell receptor beta locus (TRB) on chromosome 7
SEQ ID NO: 4: TRAC polypeptide
SEQ ID NO: 5-6 TRBC polypeptide
SEQ ID NO: 5: TRBC1
SEQ ID NO: 6: TRBC2
SEQ ID NO: 7 CD3zeta polypeptide, e.g. a modified CD3zeta polypeptide
SEQ ID NO: 8-9: CD28
SEQ ID NO: 10-11: 4-1BB
SEQ ID NO: 12: ICOS
SEQ ID NO:13: OX40
SEQ ID NO:14: CD80 co-stimulatory ligand
SEQ ID NO: 15: human SUV39H1 gene
SEQ ID NO: 16: SUV39H1 human protein sequence
SEQ ID NO: 17: TRAC exon 1
SEQ ID NO: 18: p95HER2 extracellular domain:

MPIWKFPDEEGACQPCPINCTHSCVDKDDKGCPAEQRASPLT.
SEQ ID NO: 19: exemplary linker sequence
GGGGSGGGGSGGGGS
SEQ ID NO: 20: IL-2 signal sequence (human)
MYRMQLLSCIALSLALVTNS
SEQ ID NO: 21: IL-2 signal sequence (mouse)
MYSMQLASCVTLTLVLLVNS
SEQ ID NO: 22 kappa leader sequence (human)
METPAQLLFLLLLWLPDTTG
SEQ ID NO: 23 kappa leader sequence (mouse)
METDTLLLWVLLLWVPGSTG
SEQ ID NO: 24 CD8 leader sequence
MALPVTALLLPLALLLHAARP
SEQ ID NO: 25 truncated human CD8 signal peptide:
MALPVTALLLPLALLLHA
SEQ ID NO: 26 albumin signal sequence:
MKWVTFISLLFSSAYS
SEQ ID NO: 27: prolactin signal sequence:
MDSKGSSQKGSRLLLLLVVSNLLLCQGVVS
SEQ ID NO: 28: Murine TRBC nucleotide sequence
gaggacctgcggaatgtcacgcccccgaaagtgtccctgtttgaaccaagtaaagccgagattgcgaataagcagaag
gctactctggtttgcttggcacgaggattttttcctgaccatgtggaactgagctggtgggttaatgggaaagaagttcattcag
gcgtatgtactgatccacaggcctacaaggaatcaaattactcttactgtctctcttcccgattgcgcgtttctgctacattttggc
acaatcctcgaaatcatttccggtgccaagttcaattccacggtttgtcagaggaagacaaatggcccgagggcagtccta
agccagtaacgcagaatatatcagctgaggcgtggggcagagctgactgtggtataacttcagcgagctatcagcaagg
ggttctgagtgctactatcctctacgagatcctgctagggaaggccaccctgtatgctgtgctggtctctactctcgtggttatg
gcaatggtgaaacggaaaaacagc
SEQ ID NO: 29: Murine TRBC aa sequence
EDLRNVTPPKVSLFEPSKAEIANKQKATLVCLARGFFPDHVELSWWVNGKEVHSGVC
TDPQAYKESNYSYCLSSRLRVSATFWHNPRNHFRCQVQFHGLSEEDKWPEGSPKPV
TQNISAEAWGRADCGITSASYQQGVLSATILYEILLGKATLYAVLVSTLVVMAMVKRKNS
SEQ ID NO: 30: Murine TRAC1 nucleotide sequence
atccagaaccctgagcccgctgtgtaccaattgaaagacccacggtcacaggacagtaccttgtgcctcttcacggacttt
gactctcagatcaacgttcctaagacgatggaaagtggcacatttatcacggataagtgtgtcttggatatgaaagctatgg
atagcaagtctaatggtgctatcgcatggtcaaaccagacttccttcacctgccaggatatttttaaggaaaccaatgcaact
tacccatctagcgatgtaccatgcgatgccaccctgacggagaaaagctttgaaacagatatgaacctaaactttcaaaa
cctgctggtgattgtactgcgaatcctcctcctgaaagtggccgggtttaatctgctcatgacgctgcggctgtggtccagc
SEQ ID NO: 31: Murine TRAC1 aa sequence
IQNPEPAVYQLKDPRSQDSTLCLFTDFDSQINVPKTMESGTFITDKCVLDMKAMDSKS
NGAIAWSNQTSFTCQDIFKETNATYPSSDVPCDATLTEKSFETDMNLNFQNLLVIVLRIL
LLKVAGFNLLMTLRLWSS
SEQ ID NO: 32: CD80-4-1BB booster nucleotide sequence
atgggccacacacggaggcagggaacatcaccatccaagtgtccatacctcaatttctttcagctcttggtgctggctggtct
ttctcacttctgttcaggtgttatccacgtgaccaaggaagtgaaagaagtggcaacgctgtcctgtggtcacaatgtttctgtt
gaagagctggcacaaactcgcatctactggcaaaaggagaagaaaatggtgctgactatgatgtctggggacatgaat
atatggcccgagtacaagaaccggaccatctttgatatcactaataacctctccattgtgatcctggctctgcgcccatctga
cgagggcacatacgagtgtgttgttctgaagtatgaaaaagacgctttcaagcgggaacacctggctgaagtgacgttatc
agtcaaagctgacttccctacacctagtatatctgactttgaaattccaacttctaatattagaaggataatttgctcaacctctg
gaggttttccagagcctcacctctcctggttggaaaatggagaagaattaaatgccatcaacacaacagtttcccaagatc
ctgaaactgagctctatgctgttagcagcaaactggatttcaatatgacaaccaaccacagcttcatgtgtctcatcaagtat
ggacatttaagagtgaatcagaccttcaactggaatacaaccaagcaagagcattttcctgataacctgctcccatcctgg
gccattaccttaatctcagtaaatggaatttttgtgatatgctgcctgacctactgctttaaacggggcagaaagaaactcctg
tatatattcaaacaaccatttatgagaccagtacaaactactcaagaggaagatggctgtagctgccgatttccagaagaa
gaagaaggaggatgtgaactgtga
SEQ ID NO: 33: CD80-4-1BB booster aa sequence
MGHTRRQGTSPSKCPYLNFFQLLVLAGLSHFCSGVIHVTKEVKEVATLSCGHNVSVEE
LAQTRIYWQKEKKMVLTMMSGDMNIWPEYKNRTIFDITNNLSIVILALRPSDEGTYECV
VLKYEKDAFKREHLAEVTLSVKADFPTPSISDFEIPTSNIRRIICSTSGGFPEPHLSWLEN
GEELNAINTTVSQDPETELYAVSSKLDFNMTTNHSFMCLIKYGHLRVNQTFNWNTTKQ
EHFPDNLLPSWAITLISVNGIFVICCLTYCFKRGRKKLLYIFKQPFMRPVQTTQEEDGCS
CRFPEEEEGGCEL

The sequence utilized for the AAV6 HIT for KI in the TRAC locus was described here: Mansilla-Soto et al., https://doi.org/10.1038/s41591-021-01621-1

SEQ ID NO: 34: SJ25C1 nucleotide VH
gaggtgaagctgcagcagtctggggctgagctggtgaggcctgggtcctcagtgaagatttcctgcaaggcttctggctat
gcattcagtagctactggatgaactgggtgaagcagaggcctggacagggtcttgagtggattggacagatttatcctgga
gatggtgatactaactacaatggaaagttcaagggtcaagccacactgactgcagacaaatcctccagcacagcctaca
tgcagctcagcggcctaacatctgaggactctgcggtctatttctgtgcaagaaagaccattagttcggtagtagatttctactt
tgactactggggccaagggaccacggtcaccgtc
SEQ ID NO: 35: SJ2501 nucleotide VL
gacattgagctcacccagtctccaaaattcatgtccacatcagtaggagacagggtcagcgtcacctgcaaggccagtca
gaatgtgggtactaatgtagcctggtatcaacagaaaccaggacaatctcctaaaccactgatttactcggcaacctaccg
gaacagtggagtccctgatcgAttcacaggcagtggatctgggacagatttcactctcaccatcactaacgtgcagtctaa
agacttggcagactatttctgtcaacaatataacaggtatccgtacacgtccggaggggggaccaagctggagatc
SEQ ID NO: 36: SJ2501 aa VH
EVKLQQSGAELVRPGSSVKISCKASGYAFSSYWMNWVKQRPGQGLEWIGQIYPGDG
DTNYNGKFKGQATLTADKSSSTAYMQLSGLTSEDSAVYFCARKTISSVVDFYFDYWG
QGTTVTV
SEQ ID NO: 37: SJ25C1 aa VL
DIELTQSPKFMSTSVGDRVSVTCKASQNVGTNVAWYQQKPGQSPKPLIYSATYRNSG
VPDRFTGSGSGTDFTLTITNVQSKDLADYFCQQYNRYPYTSGGGTKLEI

The sequences for gRV HITs are as described below

SEQ ID NO: 38: FMC63 nucleotide VH
gaggtgaaactgcaggagtcaggacctggcctggtggcgccctcacagagcctgtccgtcacatgcactgtctcagggg
tctcattacccgactatggtgtaagctggattcgccagcctccacgaaagggtctggagtggctgggagtaatatggggta
gtgaaaccacatactataattcagctctcaaatccagactgaccatcatcaaggacaactccaagagccaagttttcttaa
aaatgaacagtctgcaaactgatgacacagccatttactactgtgccaaacattattactacggtggtagctatgctatgga
ctactggggccaaggaacctcagtcaccgtc
SEQ ID NO: 39: FMC63 nucleotide VL
gacatccagatgacacagactacatcctccctgtctgcctctctgggagacagagtcaccatcagttgcagggcaagtca
ggacattagtaaatatttaaattggtatcagcagaaaccagatggaactgttaaactcctgattaccatacatcaagattac
actcaggagtcccatcaaggttcagtggcagtgggtctggaacagattattctctcaccattagcaacctggagcaagaag
atattgccacttactittgccaacagggtaatacgcttccgtacacgttcggaggggggaccaagctggagatc
SEQ ID NO: 40: FMC63 aa VH
EVKLQESGPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAMDYWGQGTS
VTV
SEQ ID NO: 41: FMC63 aa VL
DIQMTQTTSSLSASLGDRVTISCRASQDISKYLNWYQQKPDGTVKLLIYHTSRLHSGVP
SRFSGSGSGTDYSLTISNLEQEDIATYFCQQGNTLPYTFGGGTKLEI
SEQ ID NO: 42: mJ591 nucleotide VH
gaggtacagctacaacaatcaggacccgaattgaagaaaccgggtacatccgtgcgcatcagctgtaagacctccggtt
atacctttaccgaatataccatccattgggttaaacagagccacggcaaatcgctggaatggattggtaacatcaacccga
acaacggtggcaccacgtacaatcagaagttcgaggacaaagccaccctgacggttgacaagagctcttccactgcgt
atatggaactgcgtagcctgaccagcgaggacagcgcggtgtactactgcgcggctggttggaattttgattactggggtc
aaggtacgaccttgaccgtc
SEQ ID NO: 43: mJ591 nucleotide VL
gatattgttatgacccagagccacaaattcatgagcacctctgtgggtgatcgcgtgtccatcatctgcaaggcgagccag
gatgttggtactgccgtcgactggtaccagcaaaaaccgggtcaatccccaaaattgctgatttactgggccagcacgcgt
cataccggggtgccggatcgttttaccggttctggcagcggcactgacttcacgctgaccattaccaatgttcagtcggagg
acctggcagactacttctgccaacaatataacagctatccgctgacctttggtgcaggtacgatgttagatctt
SEQ ID NO: 44: mJ591 aa VH
EVQLQQSGPELKKPGTSVRISCKTSGYTFTEYTIHWVKQSHGKSLEWIGNINPNNGGT
TYNQKFEDKATLTVDKSSSTAYMELRSLTSEDSAVYYCAAGWNFDYWGQGTTLTV
SEQ ID NO: 45: mJ591 aa VL
DIVMTQSHKFMSTSVGDRVSIICKASQDVGTAVDWYQQKPGQSPKLLIYWASTRHTGV
PDRFTGSGSGTDFTLTITNVQSEDLADYFCQQYNSYPLTFGAGTMLDL
SEQ ID NO: 46: TRAC nucleotide
atccagaaccccgaccctgccgtctaccagctgagagattcaaaatcctccgacaaatccgtctgtctcttcaccgacttcg
actcacagacaaatgtgtcccagtccaaggatagtgacgtgtacatcaccgacaaaactgtgctggacatgagatcaatg
gatttcaaatccaactccgccgtggcatggagtaacaaatccgatttcgcctgtgccaatgccttcaacaactccatcatcc
ctgaggacaccttttttccatctcccgaatcatcttgtgacgtgaaactcgtcgagaaatcattcgaaaccgacaccaacctc
aactttcagaatctgtccgtcatcggctttaggattctgctgctgaaagtggccggattcaatctgctcatgaccctgagactgt
ggtcatct
SEQ ID NO: 47: TRAC nucleotide
atTcagaaTccTgaTcctgccgtctaccagctgagagattcaaaatcctccgacaaatccgtctgtctcttcaccgacttc
gactcacagacaaatgtgtcccagtccaaggatagtgacgtgtacatcaccgacaaaactgtgctggacatgagatcaat
ggatttcaaatccaactccgccgtggcatggagtaacaaatccgatttcgcctgtgccaatgccttcaacaactccatcatc
cctgaggacaccttttttccatctcccgaatcatcttgtgacgtgaaactcgtcgagaaatcattcgaaaccgacaccaacct
caactttcagaatctgtccgtcatcggctttaggattctgctgctgaaagtggccggattcaatctgctcatgaccctgagact
gtggtcatct
SEQ ID NO: 48: TRAC (aa)
IQNPDPAVYQLRDSKSSDKSVCLFTDFDSQTNVSQSKDSDVYITDKTVLDMRSMDFKS
NSAVAWSNKSDFACANAFNNSIIPEDTFFPSPESSCDVKLVEKSFETDTNLNFQNLSVI
GFRILLLKVAGFNLLMTLRLWSS
SEQ ID NO: 49: TRBC nucleotide
ctggaggatctgaaaaacgtgttccctcctgaagtggctgtctttgaaccatccgaggccgagatttcccatacccagaaa
gcaactctggtctgtctggccactggattctaccccgatcacgtggaactgtcttggtgggtgaacggcaaggaagtccatt
ccggagtctctaccgaccctcagcccctcaaggagcagcctgctctcaacgattctcggtactgcctgtcatctcgactgag
agtgtctgccaccttctggcagaaccctagaaaccactttcggtgtcaggtccagttttacggcctgagcgagaacgatga
gtggacacaggatagagccaaacctgtgacacagattgtgagcgcTgaggcttggggacgagccgattgtggcttcac
atccgagtcttaccagcagggagtgctgtctgctacaatcctctacgaaattctcctggggaaggccaccctgtacgctgtc
ctcgtgtctgctctggtgctcatggctatggtcaaacgaaaggactctagaggc
SEQ ID NO: 50: TRBC aa
LEDLKNVFPPEVAVFEPSEAEISHTQKATLVCLATGFYPDHVELSWWVNGKEVHSGVS
TDPQPLKEQPALNDSRYCLSSRLRVSATFWQNPRNHFRCQVQFYGLSENDEWTQDR
AKPVTQIVSAEAWGRADCGFTSESYQQGVLSATILYEILLGKATLYAVLVSALVLMAMV
KRKDSRG
SEQ ID NO: 51: P2A (nt)
Ggctctggcgctaccaatttttccctcctcaaacaggctggagatgtcgaagagaaccccggacct
SEQ ID NO: 52: CD8 alpha (nt)
Atggctctcccagtgactgccctactgcttcccctagcgcttctcctgcatgcagccaggccg
SEQ ID NO: 53: CD80 Booster (nt)
Atgggccacacacggaggcagggaacatcaccatccaagtgtccatacctcaatttctttcagctcttggtgctggctggtc
tttctcacttctgttcaggtgttatccacgtgaccaaggaagtgaaagaagtggcaacgctgtcctgtggtcacaatgtttctgt
tgaagagctggcacaaactcgcatctactggcaaaaggagaagaaaatggtgctgactatgatgtctggggacatgaat
atatggcccgagtacaagaaccggaccatctttgatatcactaataacctctccattgtgatcctggctctgcgcccatctga
cgagggcacatacgagtgtgttgttctgaagtatgaaaaagacgctttcaagcgggaacacctggctgaagtgacgttatc
agtcaaagctgacttccctacacctagtatatctgactttgaaattccaacttctaatattagaaggataatttgctcaacctctg
gaggttttccagagcctcacctctcctggttggaaaatggagaagaattaaatgccatcaacacaacagtttcccaagatc
ctgaaactgagctctatgctgttagcagcaaactggatttcaatatgacaaccaaccacagcttcatgtgtctcatcaagtat
ggacatttaagagtgaatcagaccttcaactggaatacaaccaagcaagagcattttcctgataacctgctcccatcctgg
gccattaccttaatctcagtaaatggaatttttgtgatatgctgcctgacctactgcttt
SEQ ID NO: 54: CD80 Booster (aa)
MGHTRRQGTSPSKCPYLNFFQLLVLAGLSHFCSGVIHVTKEVKEVATLSCGHNVSVEE
LAQTRIYWQKEKKMVLTMMSGDMNIWPEYKNRTIFDITNNLSIVILALRPSDEGTYECV
VLKYEKDAFKREHLAEVTLSVKADFPTPSISDFEIPTSNIRRIICSTSGGFPEPHLSWLEN
GEELNAINTTVSQDPETELYAVSSKLDFNMTTNHSFMCLIKYGHLRVNQTFNWNTTKQ
EHFPDNLLPSWAITLISVNGIFVICCLTYCF
SEQ ID NO:55: IncRNA AF196970.3 cDNA sequence
cgaggcagggctttggctactggagatcgtaggttcgaatcccgtctgggaagttcaacttgtgcacctgtaaaagaagct
ggcattattggcttgtactcaagggctggcacagagtgtgtcggggtgcggacgccccagccacgcccatcatccgtgcg
gaagatgcagaggtcatatcggatacccttctgtaccacacgatttgggcagtcatagccgcagcggcagcgggagttgc
actcgtagatgggcagcccggctcgaagccgcacctggccctggtcattgtaggcaaacttgtgcagtgacgcccccgg
gcagcagcctccagtgggtgcccacagacagtcctggcactcgcagcccacagccacctggttgagggtgatgccctca
ccaacacggtactcattgatgtacacgaaggcccgcggagggccgtccaggtccacctcattctctacagtgatgcgtcc
cagatggctgcgcttggcattgagctcctgctcccagcgacggagcgcccgcctctgcttggccttctgcaccaggtagttg
gccaagcttgggtccaggtgccggggggtctttgaccggtggtgccgccggagcagctccctttctaagtccttgtggaact
gcttgaggatacgcacacacttgagattctgccgtggctcccaggtgctctctgagtctggatatccacgccatttcaccagg
gtcaaaggagaaaattccctttggaaacagatgtgggcagttggggacaagagggcaggacactaacttccttgtgacct
gtcccctcccagagcatggtcaccccagactcacgcggatcttcttgtaatcgcacaggtactcgacttcaaagtcataga
ggttcctcttagagataccgagggcagggcaggagagc

1. A modified immune cell expressing an antigen-specific receptor that is a modified TCR that comprises a heterologous antigen-binding domain that specifically binds a target antigen, and wherein:
(a) a SUV39H1 gene of said immune cell is inactivated; and
(b) optionally, the antigen-specific receptor comprises a single active ITAM domain.

2. The modified immune cell of claim 1 wherein the immune cell comprises a nucleic acid encoding a SUV39H1 inhibitor, optionally (a) a dominant negative SUV39H1 gene or (b) an RNAi, lncRNA, shRNA, ribozyme or antisense oligonucleotide complementary to a fragment of the SUV39H1 gene.

3. The modified immune cell of any of the preceding claims wherein the SUV39H1 gene comprises one or more mutations that results in a deleted or non-functional SUV39H1 protein.

4. The modified immune cell of claim 1 wherein the SUV39H1 gene is inactivated by contacting the immune cell with an SUV39H1 inhibitor, optionally a epipolythiodioxopiperazine (ETP) class of inhibitors, such as an epidithiodioxopiperazine alkaloid, or ETP69.

5. The modified immune cell of any of the preceding claims wherein the antigen-specific receptor is a modified TCR comprising
(a) a first antigen-binding chain comprising an antigen-binding fragment optionally a heavy chain variable region (VH) of an antibody; and
(b) a second antigen-binding chain comprising an antigen-binding fragment, optionally a light chain variable region (VL) of an antibody;
wherein the first and second antigen-binding chains each comprise a TRAC polypeptide or a TRBC polypeptide, optionally wherein at least one of the TRAC polypeptide and the TRBC polypeptide is endogenous, and optionally wherein one or both of the endogenous TRAC and TRBC polypeptides is inactivated.

6. The modified immune cell of any of the preceding claims wherein the modified TCR comprises (a) VH or fragment thereof, linked to a TCR beta constant region having at least 90% sequence identity to SEQ ID NO: 5 or 6, and (b) a VL or fragment thereof, linked to a TCR alpha constant region having at least 90% sequence identity to SEQ ID NO: 4, and optionally (c) a CD3zeta polypeptide comprising a single ITAM, optionally a CD3zeta polypeptide comprising a deletion of amino acids 90-164 of SEQ ID NO: 7.

7. The modified immune cell of any of the preceding claims wherein the antigen-binding domain binds an antigen with a KD affinity of about 1×10⁻⁷ or less, about 5×10⁻⁸ or less, about 1×10⁻⁸ or less, about 5×10⁻⁹ or less, about 1×10⁻⁹ or less, about 5×10⁻¹⁰ or less, about 1×10⁻¹⁰ or less, about 5×10⁻¹¹ or less, about 1×10⁻¹¹ or less, about 5×10⁻¹² or less, or about 1×10⁻¹² or less.

8. The modified immune cell of any of the preceding claims comprising a second antigen-specific receptor that binds a second target antigen.

9. The modified immune cell of any of the preceding claims wherein the SUV39H1 cell also expresses a chimeric antigen receptor (CAR) comprising:
(a) an extracellular antigen-binding domain,
(b) a transmembrane domain,
(c) optionally one or more costimulatory domains, and
(d) optionally an intracellular signaling domain comprising a modified CD3zeta intracellular signaling domain in which ITAM2 and ITAM3 have been inactivated.

10. The modified immune cell of any of the preceding claims that further comprises a co-stimulatory receptor, optionally a receptor comprising (a) an extracellular and transmembrane domain of CD86, 41BBL, CD275, CD40L, OX40L, PD-1, TIGIT, 2B4, or NRP1, and (b) an intracellular co-stimulatory molecule of CD28, 4-1BB, OX40, ICOS, CD27, CD40, or CD2; or optionally a receptor comprising a CD80 co-stimulatory ligand (SEQ ID NO: 14) and a 4-1BB co-stimulatory molecule (SEQ ID NO: 10), optionally a receptor of SEQ ID 33.

11. The modified immune cell of any of the preceding claims wherein a nucleic acid encoding the heterologous antigen-binding domain or antigen-specific receptor is inserted into the endogenous TRAC locus and/or TRBC locus of the immune cell, optionally wherein said nucleic acid is operably linked to an endogenous TCR promoter.

12. The modified immune cell of any of the preceding claims wherein the insertion of the nucleic acid sequence encoding said antigen-specific receptor and/or said antigen-binding domain disrupts or abolishes the endogenous expression of a TCR comprising a native TCR alpha chain and/or a native TCR beta chain.

13. The modified immune cell of any of the preceding claims, wherein the antigen has a low density on the cell surface, of less than about 10,000, or less than about 5,000, or less than about 2,000 molecules per cell.

14. The modified immune cell of any of the preceding claims, wherein the cell is a T cell, a CD4+ T cell, a CD8+ T cell, a CD4+ and CD8+ T cell, a NK cell, a Treg cell, a Tm cell, a memory stem T cell (TSCM), a TCM cell, a TEM cell, a T cell progenitor, an NK cell progenitor, a pluripotent stem cell, an induced pluripotent stem cell (iPSC), a hematopoietic stem cell (HSC), an adipose derived stem cell (ADSC), or a pluripotent stem cell of myeloid or lymphoid lineage.

15. The modified immune cell of any of the preceding claims, wherein the extracellular antigen-binding domain binds to orphan tyrosine kinase receptor ROR1, tEGFR, Her2, p95HER2, LI-CAM, CD19, CD20, CD22, mesothelin, CEA, Claudin 18.2, hepatitis B surface antigen, anti-folate receptor, CD23, CD24, CD30, CD33, CD38, CD44, CD70, EGFR, EGP-2, EGP-4, EPHa2, ErbB2, 3, or 4, FcRH5, FBP, fetal acethycholine e receptor, GD2, GD3, HMW-MAA, IL-22R-alpha, IL-13R-alpha2, kdr, kappa light chain, BCMA, Lewis Y, MAGE-A1, mesothelin, MUC1, MUC16, PSCA, NKG2D Ligands, NY-ESO-1, MART-1, gplOO, oncofetal antigen, TAG72, VEGF-R2, carcinoembryonic antigen (CEA), prostate specific antigen (PSMA), estrogen receptor, progesterone receptor, ephrinB2, CD 123, CS-1, c-Met, GD-2, MAGE A3, CE7, or Wilms Tumor 1 (WT-1), or optionally the extracellular antigen-binding domain binds to any of the tumor neoantigenic peptides disclosed in Int'l Pat. Pub. No. WO 2021/043804.

16. The modified immune cell of any of the preceding claims, wherein SUV39H1 expression or SUV39H1 protein activity is reduced by at least about 50%, 60%, 70%, 75%, 80%, 85%, 90% or 95%.

17. The modified immune cell of any of the preceding claims, wherein endogenous TCR expression is reduced by at least about 75%, 80%, 85%, 90% or 95%.

18. The modified immune cell of any of the preceding claims, wherein the immune cell is autologous.

19. The modified immune cell of any of the preceding claims, wherein the immune cell is allogeneic.

20. The modified immune cell of any of the preceding claims, wherein the HLA-A locus is inactivated.

21. The modified immune cell of any of the preceding claims wherein HLA class I expression is reduced by at least about 75%, 80%, 85%, 90% or 95%.

22. A sterile pharmaceutical composition comprising the modified immune cell of any of the preceding claims.

23. A kit comprising the modified immune cell of any of the preceding claims, and a delivery device or container.

24. A method of using the modified immune cell or pharmaceutical composition or kit of any of the preceding claims to treat a patient suffering from or at risk of disease associated with the antigen, optionally cancer, by administering a therapeutically effective amount of said immune cell or pharmaceutical composition to the patient.

25. The method of claim 23 wherein the immune cell is a T-cell or NK cell and a dose of less than about 5×10⁷ cells, optionally about 10⁵ to about 10⁷ cells, is administered to the patient.

26. The method of claim 23 or 24 wherein a second therapeutic agent, optionally one or more cancer chemotherapeutic agents, cytotoxic agents, hormones, anti-angiogens, radiolabelled compounds, immunotherapy, surgery, cryotherapy, and/or radiotherapy, is administered to the patient.

27. The method of claim 23 or 24 wherein an immune checkpoint modulator is administered to the patient.

28. The method of claim 26, wherein the immune checkpoint modulator is an antibody that specifically binds to, or other inhibitor of, PD1, PDL1, CTLA4, LAG3, BTLA, OX2R, TIM-3, TIGIT, LAIR-1, PGE2 receptor, EP2/4 adenosine receptor, or A2AR.
